(19) United States
(12) Patent Application Publication
(10) Pub. No.: US 2013/0116135 A1
(43) Pub. Date: May 9, 2013

(54) Methods, Kits and Reagents for Diagnosing, Alding Diagnosis and/or Monitoring Progression of a Neurological Disorder

(71) Applicants: James Doecke, Tamborine (AU); Holly Soares, Higganum, CT (US); Simon Matthew Laws, Wanneroo (AU); Noel Garry Faux, Murrumbeena (AU)

(72) Inventors: James Doecke, Tamborine (AU); Holly Soares, Higganum, CT (US); Simon Matthew Laws, Wanneroo (AU); Noel Garry Faux, Murrumbeena (AU)

(73) Assignee: Commonweath Scientific and Industrial Research Organisation, Campbell Austrailian Capital Territory (AU)

(21) Appl. No.: 13/511,794

(22) PCT Filed: Nov. 24, 2010
(86) PCT No.: PCT/AU2010/001575

§ 371 Date: Jan. 23, 2013

(30) Foreign Application Priority Data
Nov. 24, 2009 (AU) 2009905756

(51) Int. Cl. G01N 33/68

(52) U.S. Cl. CPC G01N 33/6896
USPC 506/9; 506/18; 530/389.2; 530/389.8

(57) ABSTRACT

The present inventors have identified a panel of biomarkers present in a biological sample of an individual (e.g. blood, including serum or plasma) whose concentrations or levels are altered in individuals with a neurological disorder. Accordingly, changes in the level of any one or more of these biomarkers can be used to assess cognitive function, to diagnose or aid in the diagnosis of a neurological disorder and/or to monitor a neurological disorder in a patient (e.g., tracking disease progression in a patient and/or tracking the effect of medical or surgical therapy in the patient). Changes in the level of any one or more of these biomarkers can also be used to stratify a patient (i.e., sorting an individual with a probable diagnosis of a neurological disorder or diagnosed with a neurological disorder into different classes of the disorder) and diagnosing or aiding in the diagnosis of mild cognitive impairment (MCI) as well as diagnosing or aiding in the diagnosis of cognitive impairment.

The present invention relates generally to methods, kits and reagents for diagnosing, aiding diagnosis and/or monitoring progression of a neurological disorder in an individual, such as Alzheimer's disease. Also encompassed are methods of identifying biomarkers for use in diagnosing, aiding diagnosis and/or monitoring progression of a neurological disorder in an individual and methods of screening a candidate compound for treating and/or preventing a neurological disorder, such as Alzheimer's disease.

### BACKGROUND

Neurological disorders are a group of conditions that involve the central nervous system (brain, brainstem and cerebellum), the peripheral nervous system (including cranial nerves), and the autonomic nervous system (parts of which are located in both central and peripheral nervous system). Major branches are dementia, headache, stupor and coma, seizure, sleep disorders, trauma, infections, neoplasms, neuroophthalmology, movement disorders, demyelinating diseases, spinal cord disorders, and disorders of peripheral nerves, muscle and neuromuscular junctions. Neurological disabilities are typically associated with damage to the nervous system (including the brain and spinal cord) that results in intellectual and cognitive impairment and/or loss of some other bodily function.

Neurological disorders represent quite a diverse and chronic set of conditions that are invariably difficult to treat and are often degenerative in nature. They include, but are not limited to, Alzheimer's disease, multiple sclerosis, cerebral palsy, Parkinson's disease and neuropathy (conditions affecting the peripheral nerves). Of these, Alzheimer's disease (AD) is perhaps one of the most common causes of dementia, particularly in an aging population.

AD is typically characterised as an irreversible, progressive neurological disorder in which brain cells (neurons) deteriorate, resulting in the loss of cognitive functions, primarily memory, judgment and reasoning, movement coordination, and pattern recognition (see Mckhann et al., Neurology 34; 939 (1984)) and is the most major cause of dementia. Dementia is a typical senile disease that affects approximately 9.5 percent of the population over the age of 65 and 73 percent of those suffer from a severe form of the disorder, with adverse habitual behavior and other serious symptoms. Dementia is also the fourth most common cause of death after heart disease, stroke and lung cancer. As the population rapidly ages, it is expected that the number of dementia patients will continuously increase. According to the types of dementia, 51 percent of dementia patients suffer from Alzheimer's-type dementia, and 34 percent of dementia patients suffer from vascular dementia. The etiological factors of the remaining 15 percent are infectious diseases, metabolic diseases, etc. Alzheimer's disease and vascular dementia thus remain the most common causes of dementia and hold a majority of dementia-causing diseases.

In advanced stages of AD, all memory and mental functioning may be lost. A person with AD usually has a gradual decline in mental functions, often beginning with slight memory loss, followed by losses in the ability to maintain employment, to plan and execute familiar tasks, and to reason and exercise judgment. The ultimate cause(s) of AD is(are) still unknown, although there are several risk factors that increase a person's likelihood of developing the disorder.

Whilst there are some medications that seek to modify the symptoms of Alzheimer's disease, there are currently no disease-modifying treatments. In any event, disease-modifying treatments will likely be most effective when given before the onset of permanent brain damage. However, by the time clinical diagnosis of AD is made, extensive neuronal loss has already occurred (see Price et al., 2001, Arch Neurol 58(9):1395-402). Thus, there is a need to better diagnose those patients with a neurological disorder, such as AD, so that disease-modifying treatments can be administered at an earlier stage of disease progression.

Currently, the primary method of diagnosing dementia (e.g., AD) in living patients involves taking detailed patient histories, administering memory and psychological tests, and ruling out other explanations for memory loss, including temporary (e.g., depression or vitamin B₁₂ deficiency) or permanent (e.g., stroke) conditions. Imaging examinations are also relied upon, such as magnetic resonance imaging (MRI) and positron emission tomography (PET), which are generally performed as secondary examinations.

The main clinical feature of AD is a progressive cognitive decline leading to memory loss, language impairment and other focal cognitive deficits such as apraxia, acalculia and left-right disorientation. Patients with AD also develop impaired judgment and general problem-solving difficulties. Non-cognitive or behavioural symptoms are also common in AD, with personality changes ranging from progressive passivity to marked agitation.

Whilst such clinical diagnoses can be useful, such methods are not foolproof and a final diagnosis of AD is typically determined by pathologic findings. Two pathological characteristics observed in patients with AD at autopsy include (i) extracellular amyloid plaques and (ii) intracellular tangles in the hippocampus, cerebral cortex, and other areas of the brain essential for cognitive function.

Another obstacle in the diagnosis of AD is pinpointing the type of dementia. According to research, the accuracy of a clinical diagnosis of AD is about 50 to 82% and the accuracy of a clinical diagnosis of vascular dementia is about 40 to 80%. Such large variations remain a concern to clinicians and patients alike. Because of this, AD cannot be diagnosed with complete accuracy until after death, when autopsy reveals the disease's characteristic amyloid plaques and neurofibrillary tangles in a patient's brain. In addition, clinical diagnostic procedures are only helpful after patients have begun displaying significant, abnormal memory loss or personality changes. By then, a patient has likely to have had AD for many years.

Attempts have been made to diagnose or differentially diagnose AD by measuring the level of a target in the patient whose level specifically increases or decreases in the cerebrospinal fluid (“CSF”) of a dementia patient. With regards to biomarkers, the proteins amyloid beta and tau are probably the most well characterised to date. Studies have shown that CSF samples from AD patients contain higher than normal amounts of tau and lower than normal amounts of beta amyloid. Because these biomarkers are released into CSF, a lumbar puncture (or “spinal tap”) is required to obtain a sample for testing, which presents its own risks and possible adverse side effects. Such procedures are also accompanied by pain, discomfort and only a specialized medical institution has the facility and expertise to undertake such a procedure.

In light of the above, there is a need for an improved method of identifying those with a neurological disorder such as AD, particularly at the onset of the disease, which may assist in delaying disease progression. Consequently, there is a need in the art to identify biomarkers associated with neurological disorders such as AD so as to aid in its diagnosis.

### SUMMARY OF THE INVENTION

The present inventors have identified a collection of biomarkers, present in a biological sample of an individual (e.g. blood, including serum or plasma), whose concentrations or levels are altered in individuals with a neurological disorder, such as Alzheimer's disease (AD).

The biomarkers may be used individually or in combination for diagnosing and/or aiding in the diagnosis of neurological disorders such as AD. Thus, in one aspect of the present invention there is provided a method for the diagnosis or aiding the diagnosis of a neurological disorder in an individual by measuring the amount of one or more biomarkers in a biological sample, such as a biological fluid sample from the individual, and comparing the measured amount with a reference value for each biomarker measured. The information thus obtained may be used to aid in the diagnosis or to diagnose a neurological disorder in the individual.

Accordingly, in one aspect, the present invention provides a method of diagnosing, aiding diagnosis, stratifying an individual into one or more classes, or monitoring progression of a neurological disorder, the method comprising comparing a measured level of at least four biomarkers in a biological sample from an individual to a reference level for the at least four biomarkers, wherein the at least four biomarkers are selected from a panel of markers consisting of:

| | | |
|---|---|---|
| Cortisol | SOD—superoxide | MPO—Myeloperoxidase |
| | dismutase | |
| IGF.BP.2—insulin-like | TIMP-1—tissue inhibitor of | Neut—neutrophils |
| growth factor binding | metalloproteinase 1 | |
| protein 2 | | |
| IL.17—interleukin - 17 | Adiponectin | PCV—packed cell volume |
| Pancreatic Polypeptide | BLC—chemokine (C—X—C | Rb85—Rubidium |
| | motif) ligand | |
| ApoE | β2 Microglobin | RCC—red cell count |
| ECU—apolipoprotein E | | |
| Calcium Corrected (Ca corr = | Cancer Antigen 19.9 | rFol—red cell folate |
| Ca total + ((40 − alb) * | | |
| 0.02)) | | |
| ABeta 42 | Eotaxin | Selenium |
| Apolipoprotein E4 Allelle | MIP-1-α—chemokine (C-C | TNF.RII—Tumor necrosis |
| | motif) ligand 3 | factor receptor superfamily |
| | | member 1B |
| VCAM-1—vascular cell | alb/tpr | tPr (total protein) |
| adhesion molecule 1 | | |
| Alb—albumin | CD40—CD40 molecule | VEGF Vascular endothelial |
| | | growth factor |
| B2M—beta-2-microglobulin | Chromium isotope | ANG-2—Angiopoietin-2 |
| | 52/Chromium isotope 53 | |
| CEA—carcinoembryonic | FT3 | α-2-macroglobulin |
| antigen | | |
| EGF.R—epidermal growth | HCY—homocysteine | EGFR—Epidermal growth |
| factor receptor | | factor receptor |
| Hb—haemoglobin | IL.10—interleukin 10 | Hepatocyte Growth Factor |
| | | (HGF) |
| Zinc | MCHC—mean cell | ICAM-1—Intercellular |
| | haemoglobin concentration | adhesion molecule 1 |
| Triiodothyronine | MMP.2—matrix | TNF receptor superfamily |
| | metallopeptidase 2 (72 kDa | member 5 |
| | type IV collagenase | |

and naturally occurring variants thereof.

In another embodiment, at least two of the at least four biomarkers are selected from the group consisting of:
Cortisol
IGF.BP.2—insulin-like growth factor binding protein 2
IL.17—interleukin—17
Pancreatic Polypeptide
ApoE ECU—apolipoprotein E
Calcium Corrected (Ca corr=Ca total+((40−alb)*0.02))
ABeta 42
Apolipoprotein E4 Allelle
VCAM-1—vascular cell adhesion molecule 1
   and naturally-occurring variants thereof.

In some embodiments, where the method of the present invention relates to monitoring progression of a neurological disorder, the reference level is a measured level obtained from a biological sample from the individual at an earlier point in time.

In some embodiments, the methods of the present invention further comprises comparing a measured level of at least one other biomarker in a biological sample from the individual in combination with a measured level of the at least four biomarkers to a reference level for the at least one other biomarker and the at least four biomarkers, wherein the at least one other biomarker is selected from a panel of markers consisting of:

| | |
|---|---|
| Alb—albumin | SOD—superoxide |
| | dismutase |
| B2M—beta-2-microglobulin | TIMP-1—tissue inhibitor of |
| | metalloproteinase 1 |
| CEA—carcinoembryonic | Adiponectin |
| antigen | |
| EGF.R—epidermal growth | BLC—chemokine (C—X—C |
| factor receptor | motif) ligand |
| Hb—haemoglobin | β2 Microglobin |
| Zinc | Cancer Antigen 19.9 |
| IL.17—interleukin - 17 | Eotaxin |
| VCAM-1—vascular cell | MIP-1-α—chemokine (C-C |
| adhesion molecule 1 | motif) ligand 3 |

and naturally-occurring variants thereof.

In some embodiments, the methods of the present invention further comprises comparing a measured level of at least another biomarker marker in a biological sample in combination with a measured level of the at least four biomarkers from the individual to a reference level for the at least another biomarker and the at least four biomarkers, wherein the at least another biomarker is selected from a panel of markers consisting of:

| | |
|---|---|
| alb/tpr | MPO—Myeloperoxidase |
| CD40—CD40 molecule | Neut—neutrophils |
| Chromium isotope | PCV—packed cell volume |
| 52/Chromium isotope 53 | |
| FT3 | Rb85—Rubidium |
| HCY—homocysteine | RCC—red cell count |
| IL.10—interleukin 10 | rFol—red cell folate |
| MCHC—mean cell | Selenium |
| haemoglobin concentration | |
| MMP.2—matrix | TNF.RII—Tumor necrosis |
| metallopeptidase 2 (72 kDa | factor receptor superfamily |
| type IV collagenase | member 1B |
| EGFR—Epidermal growth | tPr (total protein) |
| factor receptor | |
| Hepatocyte Growth Factor | VEGF Vascular endothelial |
| (HGF) | growth factor |
| ICAM-1—Intercellular | ANG-2—Angiopoietin-2 |
| adhesion molecule 1 | |
| TNF receptor superfamily | α-2-macroglobulin |
| member 5 | |
| Triiodothyronine | |

and naturally-occurring variants thereof.

In some embodiments, comparing the measured level of the at least four biomarkers in the biological sample from the individual comprises comparing the measured level of at least three, four, five, six, seven, eight or nine biomarkers selected from the group consisting of:
Cortisol
IGF.BP.2—insulin-like growth factor binding protein 2
IL. 17—interleukin—17
Pancreatic Polypeptide
ApoE ECU—apolipoprotein E
Calcium Corrected (Ca corr=Ca total+((40-alb)*0.02))
ABeta 42
Apolipoprotein E4 Allelle
VCAM-1—vascular cell adhesion molecule 1
   and naturally-occurring variants thereof.

In some embodiments, the method comprises comparing measured levels of:
Cortisol or a naturally-occurring variant thereof
IGF.BP.2—insulin-like growth factor binding protein 2 or a naturally-occurring variant thereof
IL.17—interleukin—17 or a naturally-occurring variant thereof
Pancreatic Polypeptide or a naturally-occurring variant thereof
ApoE ECU—apolipoprotein E or a naturally-occurring variant thereof
Calcium Corrected (Ca corr=Ca total+((40alb)*0.02)) or a naturally-occurring variant thereof
ABeta 42 or a naturally-occurring variant thereof
Apolipoprotein E4 Allelle
VCAM−1—vascular cell adhesion molecule 1 or a naturally-occurring variant thereof

A difference (i.e., increase or decrease) in the measured level of a biomarker in a biological sample from an individual as compared to a reference level for the same biomarker is typically indicative of a neurological disorder or the severity of a neurological disorder.

In some embodiments, the biomarkers of the present invention can be used in combination with the age of an individual to aid in the diagnosing, aiding diagnosis, stratifying an individual into one or more classes, or monitoring progression of a neurological disorder.

In some embodiments of the present invention, comparing the measured level to a reference level for each biomarker measured comprises calculating a fold difference between the measured level and the reference level. In some embodiments of the present invention, the method further comprises comparing the fold difference for each biomarker measured with a minimum fold difference level. In some embodiments of the present invention, the method further comprises the step of obtaining a value for the comparison of the measured level to the reference level. Also provided herein are computer readable formats comprising values obtained by the methods, as herein described.

In some embodiments, the neurological disorder is diagnosed when a biomarker is increased or decreased about 20% to about 100% as compared to a reference level of the biomarker.

In some embodiments, the biological sample is a peripheral biological fluid sample, including, but not limited to cerebral spinal fluid, blood, serum or plasma. In some embodiments, the biological sample is plasma.

In some embodiments, the comparison of the measured value and the reference value includes calculating a fold difference between the measured value and the reference value. In some embodiments the measured value is obtained by measuring the level of the biomarker(s) in the sample, while in other embodiments the measured value is obtained from a third party. Typically, an increase or a decrease in the measured level of the at least one biomarker in a biological sample from an individual as compared to a reference level of the at least one biomarker suggests a diagnosis of a neurological disorder.

In yet another aspect of the present invention, there is provided a method of identifying at least one biomarker for use in diagnosing, aiding diagnosis and/or monitoring progression of a neurological disorder in an individual and/or stratifying an individual, the method comprising obtaining measured values from a set of biological samples for a plurality of biomarkers, wherein the set of biological samples is divisible into subsets on the basis of a neurological disorder, comparing the measured values from each subset for at least one biomarker; and identifying at least one biomarker for which the measured values are significantly different between the subsets.

In some embodiments, comparing the measured values from each subset for at least one biomarker is carried out by one or more of the statistical methods selected from the group consisting of Random Forest, Support Vector Machine, Linear Models for MicroArray data (LIMMA) and/or Significance Analyses of Microarray Data (SAM), Best First, Greedy Stepwise, Naive Bayes, Linear Forward Selection, Scatter Search, Linear Discriminant Analysis (LDA), Stepwise Logistic Regression, Receiver Operating Characteristic and Classification Trees (CT).

In yet another aspect of the present invention there is provided a method of identifying candidate agents for treatment of a neurological disorder, the method comprising assaying a prospective candidate agent for activity in modulating expression and/or activity of at least four biomarkers selected from a panel of markers consisting of:

| | | |
|---|---|---|
| Cortisol | SOD—superoxide | MPO—Myeloperoxidase |
| | dismutase | |
| IGF.BP.2—insulin-like | TIMP-1—tissue inhibitor of | Neut—neutrophils |
| growth factor binding | metalloproteinase 1 | |
| protein 2 | | |
| IL.17—interleukin - 17 | Adiponectin | PCV—packed cell volume |
| Pancreatic Polypeptide | BLC—chemokine (C—X—C | Rb85—Rubidium |
| | motif) ligand | |
| ApoE | β2 Microglobin | RCC—red cell count |
| ECU—apolipoprotein E | | |
| Calcium Corrected (Ca corr = | Cancer Antigen 19.9 | rFol—red cell folate |
| Ca total + ((40 − alb) * | | |
| 0.02)) | | |
| ABeta 42 | Eotaxin | Selenium |
| Apolipoprotein E4 Allelle | MIP-1-α—chemokine (C-C | TNF.RII—Tumor necrosis |
| | motif) ligand 3 | factor receptor superfamily |
| | | member 1B |
| VCAM-1—vascular cell | alb/tpr | tPr (total protein) |
| adhesion molecule 1 | | |
| Alb—albumin | CD40—CD40 molecule | VEGF Vascular endothelial |
| | | growth factor |
| B2M—beta-2-microglobulin | Chromium isotope | ANG-2—Angiopoietin-2 |
| | 52/Chromium isotope 53 | |
| CEA—carcinoembryonic | FT3 | α-2-macroglobulin |
| antigen | | |
| EGF.R—epidermal growth | HCY—homocysteine | EGFR—Epidermal growth |
| factor receptor | | factor receptor |
| Hb—haemoglobin | IL.10—interleukin 10 | Hepatocyte Growth Factor |
| | | (HGF) |
| Zinc | MCHC—mean cell | ICAM-1—Intercellular |
| | haemoglobin concentration | adhesion molecule 1 |
| Triiodothyronine | MMP.2—matrix | TNF receptor superfamily |
| | metallopeptidase 2 (72 kDa | member 5 |
| | type IV collagenase | |

and naturally-occurring variants thereof.

In another embodiment, at least two of the at least four biomarkers are selected from the group consisting of:
Cortisol
IGF.BP.2—insulin-like growth factor binding protein 2
IL.17—interleukin—17
Pancreatic Polypeptide
ApoE ECU—apolipoprotein E
Calcium Corrected (Ca corr=Ca total+((40−alb)*0.02))
ABeta 42
Apolipoprotein E4 Allelle
VCAM-1—vascular cell adhesion molecule 1
   and naturally-occurring variants thereof.

In some embodiments, the method further comprises assaying a candidate agent for treatment of a neurological disorder, the method comprising assaying a prospective candidate agent for activity in modulating expression and/or activity of at least one other biomarker and the at least four biomarkers wherein the other biomarker is selected from a panel of markers consisting of:

| | |
|---|---|
| Alb—albumin | SOD—superoxide |
| | dismutase |
| B2M—beta-2-microglobulin | TIMP-1—tissue inhibitor of |
| | metalloproteinase 1 |
| CEA—carcinoembryonic | Adiponectin |
| antigen | |
| EGF.R—epidermal growth | BLC—chemokine (C—X—C |
| factor receptor | motif) ligand |
| Hb—haemoglobin | β2 Microglobin |
| Zinc | Cancer Antigen 19.9 |
| IL.17—interleukin - 17 | Eotaxin |
| VCAM-1—vascular cell | MIP-1-α—chemokine (C-C |
| adhesion molecule 1 | motif) ligand 3 |

and naturally-occurring variants thereof.

In some embodiments, the method further comprises assaying a candidate agent for treatment of a neurological disorder, the method comprising assaying a prospective candidate agent for activity in modulating expression and/or activity of at least another biomarker and the at least four biomarkers wherein the another biomarker is selected from a panel of markers consisting of:

| | |
|---|---|
| alb/tpr | MPO—Myeloperoxidase |
| CD40—CD40 molecule | Neut—neutrophils |
| Chromium isotope | PCV—packed cell volume |
| 52/Chromium isotope 53 | |
| FT3 | Rb85—Rubidium |
| HCY—homocysteine | RCC—red cell count |
| IL.10—interleukin 10 | rFol—red cell folate |
| MCHC—mean cell | Selenium |
| haemoglobin concentration | |
| MMP.2—matrix | TNF.RII—Tumor necrosis |
| metallopeptidase 2 (72 kDa | factor receptor superfamily |
| type IV collagenase | member 1B |
| EGFR—Epidermal growth | tPr (total protein) |
| factor receptor | |
| Hepatocyte Growth Factor | VEGF Vascular endothelial |
| (HGF) | growth factor |
| ICAM-1—Intercellular | ANG-2—Angiopoietin-2 |
| adhesion molecule 1 | |
| TNF receptor superfamily | α-2-macroglobulin |
| member 5 | |
| Triiodothyronine | |

and naturally-occurring variants thereof.

In some other embodiments of the present invention, there is provided a method of identifying candidate agents for treatment of a neurological disorder, the method comprising assaying a candidate agent for activity in modulating expression and/or activity of at least four biomarkers and at least one other biomarker wherein the biomarkers are as described herein.

In some embodiments, the method comprises assaying a prospective candidate agent for activity in modulating expression and/or activity of at least three, four, five, six, seven, eight or nine biomarkers selected from the group consisting of:
Cortisol
IGF.BP.2—insulin-like growth factor binding protein 2
IL.17—interleukin—17
Pancreatic Polypeptide
ApoE ECU—apolipoprotein E
Calcium Corrected (Ca corr=Ca total+((40−alb)*0.02))
ABeta 42
Apolipoprotein E4 Allelle
VCAM−1—vascular cell adhesion molecule 1
   and naturally-occurring variants thereof.

In some embodiments, the method comprises assaying a prospective candidate agent for activity in modulating expression and/or activity of:
Cortisol or a naturally-occurring variant thereof
IGF.BP.2—insulin-like growth factor binding protein 2 or a naturally-occurring variant thereof
IL.17—interleukin—17 or a naturally-occurring variant thereof
Pancreatic Polypeptide or a naturally-occurring variant thereof
ApoE ECU—apolipoprotein E or a naturally-occurring variant thereof
Calcium Corrected (Ca corr=Ca total+((40−alb)*0.02)) or a naturally-occurring variant thereof
ABeta 42 or a naturally-occurring variant thereof
Apolipoprotein E4 Allelle
VCAM−1—vascular cell adhesion molecule 1 or a naturally-occurring variant thereof

The present invention also provides a kit for use in diagnosing, aiding diagnosis and/or monitoring progression of a neurological disorder in an individual and/or stratifying (i.e., sorting an individual with a probable diagnosis of a neurological disorder or diagnosed with a neurological disorder into different classes of the disorder) an individual, the kit comprising at least one reagent specific for at least four biomarkers, wherein the at least four biomarkers are selected from a panel of markers consisting of:

| | | |
|---|---|---|
| Cortisol | SOD—superoxide | MPO—Myeloperoxidase |
| | dismutase | |
| IGF.BP.2—insulin-like | TIMP-1—tissue inhibitor of | Neut—neutrophils |
| growth factor binding | metalloproteinase 1 | |
| protein 2 | | |
| IL.17—interleukin - 17 | Adiponectin | PCV—packed cell volume |
| Pancreatic Polypeptide | BLC—chemokine (C—X—C | Rb85—Rubidium |
| | motif) ligand | |
| ApoE | β2 Microglobin | RCC—red cell count |
| ECU—apolipoprotein E | | |
| Calcium Corrected (Ca corr = | Cancer Antigen 19.9 | rFol—red cell folate |
| Ca total + ((40 − alb) * | | |
| 0.02)) | | |
| ABeta 42 | Eotaxin | Selenium |
| Apolipoprotein E4 Allelle | MIP-1-α—chemokine (C-C | TNF.RII—Tumor necrosis |
| | motif) ligand 3 | factor receptor superfamily |
| | | member 1B |
| VCAM -1—vascular cell | alb/tpr | tPr (total protein) |
| adhesion molecule 1 | | |
| Alb—albumin | CD40—CD40 molecule | VEGF Vascular endothelial |
| | | growth factor |
| B2M—beta-2-microglobulin | Chromium isotope | ANG-2—Angiopoietin-2 |
| | 52/Chromium isotope 53 | |
| CEA—carcinoembryonic | FT3 | α-2-macroglobulin |
| antigen | | |
| EGF.R—epidermal growth | HCY—homocysteine | EGFR—Epidermal growth |
| factor receptor | | factor receptor |
| Hb—haemoglobin | IL.10—interleukin 10 | Hepatocyte Growth Factor |
| | | (HGF) |
| Zinc | MCHC—mean cell | ICAM-1—Intercellular |
| | haemoglobin concentration | adhesion molecule 1 |
| Triiodothyronine | MMP.2—matrix | TNF receptor superfamily |
| | metallopeptidase 2 (72 kDa | member 5 |
| | type IV collagenase | |

and naturally-occurring variants thereof.

In another embodiment, at least two of the at least four biomarkers are selected from the group consisting of:
Cortisol
IGF.BP.2—insulin-like growth factor binding protein 2
IL.17—interleukin—17
Pancreatic Polypeptide
ApoE ECU—apolipoprotein E
Calcium Corrected (Ca corr=Ca total+((40−alb)* 0.02))
ABeta 42
Apolipoprotein E4 Allelle
VCAM−1—vascular cell adhesion molecule 1
   and naturally-occurring variants thereof.

In some embodiments, the kit further comprises at least one reagent specific for at least one other biomarker in combination with the one reagent for the at least four biomarkers, wherein the at least one other biomarker is selected from a panel of markers consisting of:

| | |
|---|---|
| Alb—albumin | SOD—superoxide |
| | dismutase |
| B2M—beta-2-microglobulin | TIMP-1—tissue inhibitor of |
| | metalloproteinase 1 |
| CEA—carcinoembryonic | Adiponectin |
| antigen | |
| EGF.R—epidermal growth | BLC—chemokine (C—X—C |
| factor receptor | motif) ligand |
| Hb—haemoglobin | β2 Microglobin |
| Zinc | Cancer Antigen 19.9 |
| IL.17—interleukin-17 | Eotaxin |
| VCAM-1—vascular cell | MIP-1-α—chemokine (C—C |
| adhesion molecule 1 | motif) ligand 3 |

and naturally-occurring variants thereof.

In some embodiments, the kit further comprises at least one reagent specific for at least another biomarker in combination the one reagent for the at least four biomarkers, wherein the at least another biomarker is selected from a panel of markers consisting of:

| | |
|---|---|
| alb/tpr | MPO—Myeloperoxidase |
| CD40—CD40 molecule | Neut—neutrophils |
| Chromium isotope | PCV—packed cell volume |
| 52/Chromium isotope 53 | |
| FT3 | Rb85—Rubidium |
| HCY—homocysteine | RCC—red cell count |
| IL.10—interleukin 10 | rFol—red cell folate |
| MCHC—mean cell | Selenium |
| haemoglobin concentration | |
| MMP.2—matrix | TNF.RII—Tumor necrosis |
| metallopeptidase 2 (72 kDa | factor receptor superfamily |
| type IV collagenase | member 1B |
| EGFR—Epidermal growth | tPr (total protein) |
| factor receptor | |
| Hepatocyte Growth Factor | VEGF Vascular endothelial |
| (HGF) | growth factor |
| ICAM-1—Intercellular | ANG-2—Angiopoietin-2 |
| adhesion molecule 1 | |
| TNF receptor superfamily | α-2-macroglobulin |
| member 5 | |
| Triiodothyronine | |

and naturally-occurring variants thereof.

In some other embodiments, the present invention provides a kit for use in diagnosing, aiding diagnosis and/or monitoring progression of a neurological disorder in an individual and/or stratifying (i.e., sorting an individual with a probable diagnosis of a neurological disorder or diagnosed with a neurological disorder into different classes of the disorder) an individual, the kit comprising at least one reagent specific for at least four biomarkers, and at least one reagent specific for at least one other and another marker wherein the markers are as described herein.

In some embodiments, the kit comprises at least one reagent specific for at least three, four, five, six, seven, eight or nine biomarkers selected from the panel of markers consisting of:
Cortisol
IGF.BP.2—insulin-like growth factor binding protein 2
IL.17—interleukin—17
Pancreatic Polypeptide
ApoE ECU—apolipoprotein E
Calcium Corrected (Ca corr=Ca total+((40−alb)* 0.02))
ABeta 42
Apolipoprotein E4 Allelle
VCAM−1—vascular cell adhesion molecule 1
   and naturally-occurring variants thereof.

In some embodiments, the kit comprises at least one reagent specific for:
Cortisol or a naturally-occurring variant thereof
IGF.BP.2—insulin-like growth factor binding protein 2 or a naturally-occurring variant thereof
IL.17—interleukin—17 or a naturally-occurring variant thereof
Pancreatic Polypeptide or a naturally-occurring variant thereof
ApoE ECU—apolipoprotein E or a naturally-occurring variant thereof
Calcium Corrected (Ca corr=Ca total+((40−alb)*0.02)) or a naturally-occurring variant thereof
ABeta 42 or a naturally-occurring variant thereof
Apolipoprotein E4 Allelle
VCAM−1—vascular cell adhesion molecule 1 or a naturally-occurring variant thereof

In some embodiments, the kit comprises at least one reference biomarker, wherein the reference biomarker is selected from the group consisting of:

| | | |
|---|---|---|
| Cortisol | SOD—superoxide | MPO—Myeloperoxidase |
| | dismutase | |
| IGF.BP.2—insulin-like | TIMP-1—tissue inhibitor of | Neut—neutrophils |
| growth factor binding | metalloproteinase 1 | |
| protein 2 | | |
| IL.17—interleukin-17 | Adiponectin | PCV—packed cell volume |
| Pancreatic Polypeptide | BLC—chemokine | Rb85—Rubidium |
| | (C—X—C motif) ligand | |
| ApoE ECU— | β2 Microglobin | RCC—red cell count |
| apolipoprotein E | | |
| Calcium Corrected (Ca corr = | Cancer Antigen 19.9 | rFol—red cell folate |
| Ca total + ((40 − alb) * | | |
| 0.02)) | | |
| ABeta 42 | Eotaxin | Selenium |
| Apolipoprotein E4 Allelle | MIP-1-α—chemokine | TNF.RII—Tumor necrosis |
| | (C—C motif) ligand 3 | factor receptor superfamily |
| | | member 1B |
| VCAM-1—vascular cell | alb/tpr | tPr (total protein) |
| adhesion molecule 1 | | |
| Alb—albumin | CD40—CD40 molecule | VEGF Vascular endothelial |
| | | growth factor |
| B2M—beta-2-microglobulin | Chromium isotope | ANG-2—Angiopoietin-2 |
| | 52/Chromium isotope 53 | |
| CEA—carcinoembryonic | FT3 | α-2-macroglobulin |
| antigen | | |
| EGF.R—epidermal growth | HCY—homocysteine | EGFR—Epidermal growth |
| factor receptor | | factor receptor |
| Hb—haemoglobin | IL.10—interleukin 10 | Hepatocyte Growth Factor |
| | | (HGF) |
| Zinc | MCHC—mean cell | ICAM-1—Intercellular |
| | haemoglobin concentration | adhesion molecule 1 |
| Triiodothyronine | MMP.2—matrix | TNF receptor superfamily |
| | metallopeptidase 2 (72 kDa | member 5 |
| | type IV collagenase | |

and naturally-occurring variants thereof.

In some embodiments, the kit further comprises instructions for carrying out the method of diagnosing and/or aiding in the diagnosis of a neurological disorder in an individual and/or monitoring progression of a neurological disorder in an individual and/or stratifying an individual (i.e., sorting an individual with a probable diagnosis of a neurological disorder or diagnosed with a neurological disorder into different classes of the disorder), as herein described.

In some embodiments, the reagent specific for the biomarker is an antibody, or a fragment thereof, capable of detecting the biomarker. In some embodiments, the kit of the present invention includes a surface to which at least one reagent specific for said biomarker is attached. In some embodiments, the kit of the present invention includes a combination of a surface as herein described having attached thereto at least one reagent specific for a biomarker and a reference sample to which a test sample can be compared. The reference sample may be a biological sample from an individual (or a pooled sample from group of individuals) with a confirmed neurological disorder.

The present invention also provides a composition for use in diagnosing, aiding diagnosis and/or monitoring progression of a neurological disorder in an individual and/or stratifying an individual, the composition comprising at least one reagent specific for at least four biomarkers, wherein the at least four biomarkers are selected from a primary panel of markers consisting of:

| | | |
|---|---|---|
| Cortisol | SOD—superoxide | MPO—Myeloperoxidase |
| | dismutase | |
| IGF.BP.2—insulin-like | TIMP-1—tissue inhibitor of | Neut—neutrophils |
| growth factor binding | metalloproteinase 1 | |
| protein 2 | | |
| IL.17—interleukin-17 | Adiponectin | PCV—packed cell volume |
| Pancreatic Polypeptide | BLC—chemokine (C—X—C | Rb85—Rubidium |
| | motif) ligand | |
| ApoE ECU— | β2 Microglobin | RCC—red cell count |
| apolipoprotein E | | |
| Calcium Corrected (Ca corr = | Cancer Antigen 19.9 | rFol—red cell folate |
| Ca total + ((40 − alb) * | | |
| 0.02)) | | |
| ABeta 42 | Eotaxin | Selenium |
| Apolipoprotein E4 Allelle | MIP-1-α—chemokine (C—C | TNF.RII—Tumor necrosis |
| | motif) ligand 3 | factor receptor superfamily |
| | | member 1B |
| VCAM-1—vascular cell | alb/tpr | tPr (total protein) |
| adhesion molecule 1 | | |
| Alb—albumin | CD40—CD40 molecule | VEGF Vascular endothelial |
| | | growth factor |
| B2M—beta-2-microglobulin | Chromium isotope | ANG-2—Angiopoietin-2 |
| | 52/Chromium isotope 53 | |
| CEA—carcinoembryonic | FT3 | α-2-macroglobulin |
| antigen | | |
| EGF.R—epidermal growth | HCY—homocysteine | EGFR—Epidermal growth |
| factor receptor | | factor receptor |
| Hb—haemoglobin | IL.10—interleukin 10 | Hepatocyte Growth Factor |
| | | (HGF) |
| Zinc | MCHC—mean cell | ICAM-1—Intercellular |
| | haemoglobin concentration | adhesion molecule 1 |
| Triiodothyronine | MMP.2—matrix | TNF receptor superfamily |
| | metallopeptidase 2 (72 kDa | member 5 |
| | type IV collagenase | |

and naturally-occurring variants thereof.

In another embodiment, at least two of the at least four biomarkers are selected from the group consisting of:
Cortisol
IGF.BP.2—insulin-like growth factor binding protein 2
IL.17—interleukin—17
Pancreatic Polypeptide
ApoE ECU—apolipoprotein E
Calcium Corrected (Ca corr=Ca total+((40−alb)*0.02))
ABeta 42
Apolipoprotein E4 Allelle
VCAM-1—vascular cell adhesion molecule 1
   and naturally-occurring variants thereof.

In some embodiments, the composition further comprises at least one reagent specific for at least one other biomarker in combination the at least one reagent specific for the at least four biomarkers, wherein the at least one other biomarker is selected from a panel of markers consisting of:

| | |
|---|---|
| Alb—albumin | SOD—superoxide |
| | dismutase |
| B2M—beta-2-microglobulin | TIMP-1—tissue inhibitor of |
| | metalloproteinase 1 |
| CEA—carcinoembryonic | Adiponectin |
| antigen | |
| EGF.R—epidermal growth | BLC—chemokine (C—X—C |
| factor receptor | motif) ligand |
| Hb—haemoglobin | β2 Microglobin |
| Zinc | Cancer Antigen 19.9 |
| IL.17—interleukin-17 | Eotaxin |
| VCAM-1—vascular cell | MIP-1-α—chemokine (C—C |
| adhesion molecule 1 | motif) ligand 3 |

and naturally-occurring variants thereof.

In some embodiments, the composition further comprises at least one reagent specific for at least another biomarker in combination with the at least one reagent for the at least four biomarkers, wherein the at least another biomarker is selected from a panel of markers consisting of:

| | |
|---|---|
| alb/tpr | MPO—Myeloperoxidase |
| CD40—CD40 molecule | Neut—neutrophils |
| Chromium isotope | PCV—packed cell volume |
| 52/Chromium isotope 53 | |
| FT3 | Rb85—Rubidium |
| HCY—homocysteine | RCC—red cell count |
| IL.10—interleukin 10 | rFol—red cell folate |
| MCHC—mean cell | Selenium |
| haemoglobin concentration | |
| MMP.2—matrix | TNF.RII—Tumor necrosis |
| metallopeptidase 2 (72 kDa | factor receptor superfamily |
| type IV collagenase | member 1B |
| EGFR—Epidermal growth | tPr (total protein) |
| factor receptor | |
| Hepatocyte Growth Factor | VEGF Vascular endothelial |
| (HGF) | growth factor |
| ICAM-1—Intercellular | ANG-2—Angiopoietin-2 |
| adhesion molecule 1 | |
| TNF receptor superfamily | α-2-macroglobulin |
| member 5 | |
| Triiodothyronine | |

and naturally-occurring variants thereof.

In some other embodiments, the present invention provides a composition for use in diagnosing, aiding diagnosis and/or monitoring progression of a neurological disorder in an individual and/or stratifying (i.e., sorting an individual with a probable diagnosis of a neurological disorder or diagnosed with a neurological disorder into different classes of the disorder) an individual, the kit comprising at least one reagent specific for at least four biomarkers, and at least one reagent specific for at least one of the other and another marker wherein the markers are as described herein.

In some embodiments, the composition further comprises at least one reagent specific for at least three, four, five, six, seven, eight or nine biomarkers selected from the panel of markers consisting of:
Cortisol
IGF.BP.2—insulin-like growth factor binding protein 2
IL.17—interleukin—17
Pancreatic Polypeptide
ApoE ECU—apolipoprotein E
Calcium Corrected (Ca corr=Ca total+((40−alb)* 0.02))
ABeta 42
Apolipoprotein E4 Allelle
VCAM−1—vascular cell adhesion molecule 1
   and naturally-occurring variants thereof.

In some embodiments, the composition comprises at least one reagent specific for:
Cortisol or a naturally-occurring variant thereof
IGF.BP.2—insulin-like growth factor binding protein 2 or a naturally-occurring variant thereof
IL.17—interleukin—17 or a naturally-occurring variant thereof
Pancreatic Polypeptide or a naturally-occurring variant thereof
ApoE ECU—apolipoprotein E or a naturally-occurring variant thereof
Calcium Corrected (Ca corr=Ca total+((40−alb)*0.02)) or a naturally-occurring variant thereof
ABeta 42 or a naturally-occurring variant thereof
Apolipoprotein E4 Allelle
VCAM−1—vascular cell adhesion molecule 1 or a naturally-occurring variant thereof

In another embodiment, at least two of the at least four biomarkers are selected from the group consisting of:
Cortisol
IGF.BP.2—insulin-like growth factor binding protein 2
IL.17—interleukin—17
Pancreatic Polypeptide
ApoE ECU—apolipoprotein E
Calcium Corrected (Ca corr=Ca total+((40−alb)*0.02))
ABeta 42
Apolipoprotein E4 Allelle
VCAM-1—vascular cell adhesion molecule 1
   and naturally-occurring variants thereof.

The present invention also provides a system of diagnosing or aiding diagnosis of a neurological disorder and/or monitoring a neurological disorder, the system comprising a computational means for comparing a measured level of at least four biomarkers in a biological sample from an individual to a reference level for the at least four biomarkers, wherein the at least four biomarkers are selected from a panel of markers consisting of:

| | | |
|---|---|---|
| Cortisol | SOD—superoxide | MPO—Myeloperoxidase |
| | dismutase | |
| IGF.BP.2—insulin-like | TIMP-1—tissue inhibitor of | Neut—neutrophils |
| growth factor binding | metalloproteinase 1 | |
| protein 2 | | |
| IL.17—interleukin-17 | Adiponectin | PCV—packed cell volume |
| Pancreatic Polypeptide | BLC—chemokine (C—X—C | Rb85—Rubidium |
| | motif) ligand | |
| ApoE ECU— | β2 Microglobin | RCC—red cell count |
| apolipoprotein E | | |
| Calcium Corrected (Ca corr = | Cancer Antigen 19.9 | rFol—red cell folate |
| Ca total + ((40 − alb) * | | |
| 0.02)) | | |
| ABeta 42 | Eotaxin | Selenium |
| Apolipoprotein E4 Allelle | MIP-1-α—chemokine (C—C | TNF.RII—Tumor necrosis |
| | motif) ligand 3 | factor receptor superfamily |
| | | member 1B |
| VCAM-1—vascular cell | alb/tpr | tPr (total protein) |
| adhesion molecule 1 | | |
| Alb—albumin | CD40—CD40 molecule | VEGF Vascular endothelial |
| | | growth factor |
| B2M—beta-2-microglobulin | Chromium isotope | ANG-2—Angiopoietin-2 |
| | 52/Chromium isotope 53 | |
| CEA—carcinoembryonic | FT3 | α-2-macroglobulin |
| antigen | | |
| EGF.R—epidermal growth | HCY—homocysteine | EGFR—Epidermal growth |
| factor receptor | | factor receptor |
| Hb—haemoglobin | IL.10—interleukin 10 | Hepatocyte Growth Factor |
| | | (HGF) |
| Zinc | MCHC—mean cell | ICAM-1—Intercellular |
| | haemoglobin concentration | adhesion molecule 1 |
| Triiodothyronine | MMP.2—matrix | TNF receptor superfamily |
| | metallopeptidase 2 (72 kDa | member 5 |
| | type IV collagenase | |

and naturally-occurring variants thereof.

The present invention also provides a method of treating an individual for a neurological disorder, the method comprising obtaining a biological sample from an individual; comparing a measured level of at least four biomarkers in the biological sample to a reference level for the at least four biomarkers, wherein the at least four biomarkers are selected from a panel of markers consisting of:

| | | |
|---|---|---|
| Cortisol | SOD—superoxide | MPO—Myeloperoxidase |
| | dismutase | |
| IGF.BP.2—insulin-like | TIMP-1—tissue inhibitor of | Neut—neutrophils |
| growth factor binding | metalloproteinase 1 | |
| protein 2 | | |
| IL.17—interleukin-17 | Adiponectin | PCV—packed cell volume |
| Pancreatic Polypeptide | BLC—chemokine (C—X—C | Rb85—Rubidium |
| | motif) ligand | |
| ApoE ECU— | β2 Microglobin | RCC—red cell count |
| apolipoprotein E | | |
| Calcium Corrected (Ca corr = | Cancer Antigen 19.9 | rFol—red cell folate |
| Ca total + ((40 − alb) * | | |
| 0.02)) | | |
| ABeta 42 | Eotaxin | Selenium |
| Apolipoprotein E4 Allelle | MIP-1-α—chemokine (C—C | TNF.RII—Tumor necrosis |
| | motif) ligand 3 | factor receptor superfamily |
| | | member 1B |
| VCAM-1—vascular cell | alb/tpr | tPr (total protein) |
| adhesion molecule 1 | | |
| Alb—albumin | CD40—CD40 molecule | VEGF Vascular endothelial |
| | | growth factor |
| B2M—beta-2-microglobulin | Chromium isotope | ANG-2—Angiopoietin-2 |
| | 52/Chromium isotope 53 | |
| CEA—carcinoembryonic | FT3 | α-2-macroglobulin |
| antigen | | |
| EGF.R—epidermal growth | HCY—homocysteine | EGFR—Epidermal growth |
| factor receptor | | factor receptor |
| Hb—haemoglobin | IL.10—interleukin 10 | Hepatocyte Growth Factor |
| | | (HGF) |
| Zinc | MCHC—mean cell | ICAM-1—Intercellular |
| | haemoglobin concentration | adhesion molecule 1 |
| Triiodothyronine | MMP.2—matrix | TNF receptor superfamily |
| | metallopeptidase 2 (72 kDa | member 5 |
| | type IV collagenase | |

and naturally-occurring variants thereof;
and, where there is a difference in the measured level of the at least four biomarkers compared to the reference level of the at least four biomarkers, indicative of a neurological disorder or severity of a neurological disorder, administering to the individual a therapeutically effective amount of an agent capable of alleviating a symptom of the neurological disorder.

### DESCRIPTION OF THE DRAWING

FIG. 1 shows the Receiver Operating Characteristic curves for predictive models developed using Random Forests, Boosted trees and Linear Discrimination Analysis.

### DETAILED DESCRIPTION OF THE INVENTION

### Method of Aiding Diagnosis of or Diagnosing a Neurological Disorder

The present inventors have identified a panel of biomarkers present in a biological sample of an individual (e.g. blood, including serum or plasma) whose concentrations or levels are altered in individuals with a neurological disorder. Accordingly, changes in the level of any one or more of these biomarkers can be used to assess cognitive function, to diagnose or aid in the diagnosis of a neurological disorder and/or to monitor a neurological disorder in a patient (e.g., tracking disease progression in a patient and/or tracking the effect of medical or surgical therapy in the patient). Changes in the level of any one or more of these biomarkers can also be used to stratify a patient (i.e., sorting an individual with a probable diagnosis of a neurological disorder or diagnosed with a neurological disorder into different classes of the disorder) and diagnosing or aiding in the diagnosis of mild cognitive impairment (MCI) as well as diagnosing or aiding in the diagnosis of cognitive impairment.

Thus, in one aspect, the present invention provides a method of aiding diagnosis of a neurological disorder, the method comprising comparing a measured level of at least four biomarkers in a biological sample from an individual to a reference level for the at least four biomarkers, wherein the at least four biomarkers are selected from a panel of markers consisting of:

| | | |
|---|---|---|
| Cortisol | SOD—superoxide | MPO—Myeloperoxidase |
| | dismutase | |
| IGF.BP.2—insulin-like | TIMP-1—tissue inhibitor of | Neut—neutrophils |
| growth factor binding | metalloproteinase 1 | |
| protein 2 | | |
| IL.17—interleukin-17 | Adiponectin | PCV—packed cell volume |
| Pancreatic Polypeptide | BLC—chemokine (C—X—C | Rb85—Rubidium |
| | motif) ligand | |
| ApoE ECU— | β2 Microglobin | RCC—red cell count |
| apolipoprotein E | | |
| Calcium Corrected (Ca corr = | Cancer Antigen 19.9 | rFol—red cell folate |
| Ca total + ((40 − alb) * | | |
| 0.02)) | | |
| ABeta 42 | Eotaxin | Selenium |
| Apolipoprotein E4 Allelle | MIP-1-α—chemokine (C—C | TNF.RII—Tumor necrosis |
| | motif) ligand 3 | factor receptor superfamily |
| | | member 1B |
| VCAM-1—vascular cell | alb/tpr | tPr (total protein) |
| adhesion molecule 1 | | |
| Alb—albumin | CD40—CD40 molecule | VEGF Vascular endothelial |
| | | growth factor |
| B2M—beta-2-microglobulin | Chromium isotope | ANG-2—Angiopoietin-2 |
| | 52/Chromium isotope 53 | |
| CEA—carcinoembryonic | FT3 | α-2-macroglobulin |
| antigen | | |
| EGF.R—epidermal growth | HCY—homocysteine | EGFR—Epidermal growth |
| factor receptor | | factor receptor |
| Hb—haemoglobin | IL.10—interleukin 10 | Hepatocyte Growth Factor |
| | | (HGF) |
| Zinc | MCHC—mean cell | ICAM-1—Intercellular |
| | haemoglobin concentration | adhesion molecule 1 |
| Triiodothyronine | MMP.2—matrix | TNF receptor superfamily |
| | metallopeptidase 2 (72 kDa | member 5 |
| | type IV collagenase | |

and naturally-occurring variants thereof.

In another embodiment, at least two of the at least four biomarkers are selected from the group consisting of:
Cortisol
IGF.BP.2—insulin-like growth factor binding protein 2
IL.17—interleukin—17
Pancreatic Polypeptide
ApoE ECU—apolipoprotein E
Calcium Corrected (Ca corr=Ca total+((40−alb)*0.02))
ABeta 42
Apolipoprotein E4 Allelle
VCAM-1—vascular cell adhesion molecule 1
   and naturally-occurring variants thereof.

In another aspect of the present invention, there is provided a method of diagnosing a neurological disorder, the method comprising comparing a measured level of at least four biomarkers in a biological sample from an individual to a reference level for the at least four biomarkers, wherein the at least four biomarkers are selected from a panel of markers consisting of:

| | | |
|---|---|---|
| Cortisol | SOD—superoxide | MPO—Myeloperoxidase |
| | dismutase | |
| IGF.BP.2—insulin-like | TIMP-1—tissue inhibitor of | Neut—neutrophils |
| growth factor binding | metalloproteinase 1 | |
| protein 2 | | |
| IL.17—interleukin-17 | Adiponectin | PCV—packed cell volume |
| Pancreatic Polypeptide | BLC—chemokine (C—X—C | Rb85—Rubidium |
| | motif) ligand | |
| ApoE ECU— | β2 Microglobin | RCC—red cell count |
| apolipoprotein E | | |
| Calcium Corrected (Ca corr = | Cancer Antigen 19.9 | rFol—red cell folate |
| Ca total + ((40 − alb) * | | |
| 0.02)) | | |
| ABeta 42 | Eotaxin | Selenium |
| Apolipoprotein E4 Allelle | MIP-1-α—chemokine (C—C | TNF.RII—Tumor necrosis |
| | motif) ligand 3 | factor receptor superfamily |
| | | member 1B |
| VCAM-1—vascular cell | alb/tpr | tPr (total protein) |
| adhesion molecule 1 | | |
| Alb—albumin | CD40—CD40 molecule | VEGF Vascular endothelial |
| | | growth factor |
| B2M—beta-2-microglobulin | Chromium isotope | ANG-2—Angiopoietin-2 |
| | 52/Chromium isotope 53 | |
| CEA—carcinoembryonic | FT3 | α-2-macroglobulin |
| antigen | | |
| EGF.R—epidermal growth | HCY—homocysteine | EGFR—Epidermal growth |
| factor receptor | | factor receptor |
| Hb—haemoglobin | IL.10—interleukin 10 | Hepatocyte Growth Factor |
| | | (HGF) |
| Zinc | MCHC—mean cell | ICAM-1—Intercellular |
| | haemoglobin concentration | adhesion molecule 1 |
| Triiodothyronine | MMP.2—matrix | TNF receptor superfamily |
| | metallopeptidase 2 (72 kDa | member 5 |
| | type IV collagenase | |

and naturally-occurring variants thereof.

For the purpose of brevity, some of the following description will be made in the context of Alzheimer's disease (AD). However, the skilled addressee would understand that the present invention may also be used in diagnosing, aiding in diagnosis and/or monitoring the progression of other neurological disorders, as well as stratifying patients according to the severity of other neurological disorders, such as those associated with neural degeneration, including, but not limited to, PD, frontotemporal dementia, cerebrovascular disease, multiple sclerosis and neuropathies. The biomarkers of the present invention can also be used to assess cognitive function in AD and other neurological disorders.

As used herein, the term “biomarker” includes, but is not limited to, proteins (polypeptides), polynucleotides and/or metabolites present in a biological sample (e.g., a biological fluid sample) whose level (e.g., concentration, expression and/or activity) in a biological sample from a subject with a neurological disorder is increased or decreased as compared to the level of the same biomarker in a normal control subject. Any listed biomarkers also include thier gene and protein synonyms.

In some embodiments, the methods of the present invention further comprises comparing a measured level of at least one other biomarker in a biological sample from the individual in combination with a measured level of the at least four biomarkers to a reference level for the at least one other biomarker and the at least four biomarkers, wherein the at least one other biomarker is selected from a panel of markers consisting of:

| | |
|---|---|
| Alb—albumin | SOD—superoxide |
| | dismutase |
| B2M—beta-2-microglobulin | TIMP-1—tissue inhibitor of |
| | metalloproteinase 1 |
| CEA—carcinoembryonic | Adiponectin |
| antigen | |
| EGF.R—epidermal growth | BLC—chemokine (C—X—C |
| factor receptor | motif) ligand |
| Hb—haemoglobin | β2 Microglobin |
| Zinc | Cancer Antigen 19.9 |
| IL.17—interleukin-17 | Eotaxin |
| VCAM-1—vascular cell | MIP-1-α—chemokine (C—C |
| adhesion molecule 1 | motif) ligand 3 |

and naturally-occurring variants thereof.

In some embodiments, the methods of the present invention further comprise comparing a measured level of at least another biomarker marker in a biological sample from the individual in combination with either or both of (i) a measured level of the at least four biomarkers and (ii) a measured level of the at least one other biomarker, to a reference level for the at least four biomarkers and the at least another biomarker and/or the at least one other biomarker, wherein the at least another biomarker is selected from a tertiary panel of markers consisting of:

| | |
|---|---|
| alb/tpr | MPO—Myeloperoxidase |
| CD40—CD40 molecule | Neut—neutrophils |
| Chromium isotope | PCV—packed cell volume |
| 52/Chromium isotope 53 | |
| FT3 | Rb85—Rubidium |
| HCY—homocysteine | RCC—red cell count |
| IL.10—interleukin 10 | rFol—red cell folate |
| MCHC—mean cell | Selenium |
| haemoglobin concentration | |
| MMP.2—matrix | TNF.RII—Tumor necrosis |
| metallopeptidase 2 (72 kDa | factor receptor superfamily |
| type IV collagenase | member 1B |
| EGFR—Epidermal growth | tPr (total protein) |
| factor receptor | |
| Hepatocyte Growth Factor | VEGF Vascular endothelial |
| (HGF) | growth factor |
| ICAM-1—Intercellular | ANG-2—Angiopoietin-2 |
| adhesion molecule 1 | |
| TNF receptor superfamily | α-2-macroglobulin |
| member 5 | |
| Triiodothyronine | |

and naturally-occurring variants thereof.

In some other embodiments, the present invention provides a method of aiding diagnosis of a neurological disorder, the method comprising comparing a measured level of at least four biomarkers in a biological sample from an individual in combination with a other and a tertiary marker to a reference level for the at least four biomarkers, and at least one of the other and another marker wherein the markers are as described herein.

In some embodiments, comparing the measured level of the at least four biomarkers in the biological sample from the individual comprises comparing the measured level of at least three three, four, five, six, seven, eight or nine biomarkers selected from the group consisting of:
Cortisol
IGF.BP.2—insulin-like growth factor binding protein 2
IL.17—interleukin—17
Pancreatic Polypeptide
ApoE ECU—apolipoprotein E
Calcium Corrected (Ca corr=Ca total+((40−alb)*0.02))
ABeta 42
Apolipoprotein E4 Allelle
VCAM−1—vascular cell adhesion molecule 1
   and naturally-occurring variants thereof.

It would be understood by the skilled addressee that the degree of sensitivity and/or selectivity of the methods of the present invention in aiding diagnosis, diagnosing and/or monitoring an individual with a neurological disorder and/or stratifying an individual, as herein described, will generally be greater where the method comprises comparing the measured level of all five biomarkers in the biological sample.

It would also be understood by the skilled addressee that the degree of sensitivity and/or selectivity of the methods of the present invention in aiding diagnosis, diagnosing and/or monitoring an individual with a neurological disorder and/or stratifying an individual, as herein described, will generally be greater where the method comprises comparing the measured level of all nine biomarkers in the biological sample with at least one other and/or at least another biomarker, as herein described.

In some embodiments of the present invention, the method of the present invention comprises comparing the measured level of:
Cortisol or a naturally-occurring variant thereof
IGF.BP.2—insulin-like growth factor binding protein 2 or a naturally-occurring variant thereof
IL.17—interleukin—17 or a naturally-occurring variant thereof
Pancreatic Polypeptide or a naturally-occurring variant thereof
ApoE ECU—apolipoprotein E or a naturally-occurring variant thereof
Calcium Corrected (Ca corr=Ca total+((40−alb)*0.02)) or a naturally-occurring variant thereof
ABeta 42 or a naturally-occurring variant thereof
Apolipoprotein E4 Allelle
VCAM−1—vascular cell adhesion molecule 1 or a naturally-occurring variant thereof
   in the biological sample of the individual to reference levels for the biomarkers.

Typically, a change in the measured level of a biomarker in a biological sample from the individual as compared to a reference level for the same biomarker is indicative of a neurological disorder.

In some embodiments, the biomarkers of the present invention can be used in combination with the age of an individual to aid in the diagnosis and/or to diagnose a neurological disorder in an individual, for example, as herein described (e.g., as described in the Examples section herein).

In some embodiments of the present invention, comparing the measured level to a reference level for each biomarker measured comprises calculating a fold difference between the measured level and the reference level. In some embodiments of the present invention, the method further comprises comparing the fold difference for each biomarker measured with a minimum fold difference level. In some embodiments of the present invention, the method further comprises the step of obtaining a value for the comparison of the measured level to the reference level. Also provided herein are computer readable formats comprising values obtained by the methods, as herein described.

In some embodiments, the neurological disorder is diagnosed when the level of a biomarker (e.g., concentration, expression and/or activity) is increased or decreased about 20% to about 100% as compared to a reference level of the biomarker.

In some embodiments, the biological sample is a peripheral biological fluid sample, including, but not limited to cerebral spinal fluid, blood, serum or plasma. In some embodiments, the biological sample is plasma.

In some embodiments, the comparison of the measured value and the reference value includes calculating a fold difference between the measured value and the reference value. In some embodiments the measured value is obtained by measuring the level of the biomarker(s) in the sample, while in other embodiments the measured value is obtained from a third party. Typically, an increase or a decrease in the measured level of the at least one biomarker in a biological sample from an individual as compared to a reference level of the at least one biomarker suggests a diagnosis of a neurological disorder.

As used herein, the terms “Alzheimer's disease patient”, “AD patient”, and “individual diagnosed with AD” and the like refer collectively to an individual who has been diagnosed with AD or has been given a probable diagnosis of AD.

As used herein, the term “biological sample” typically refers to a variety of sample types obtained from an individual that can be used in a diagnostic or monitoring assay and includes, but is not limited to, blood (including whole blood), plasma or serum, urine, cerebrospinal fluid, tears or saliva. A blood sample may include, for example, various cell types present in the blood including platelets, lymphocytes, polymorphonuclear cells, macrophages, erythrocytes. In some embodiments of the present invention, the biomarker is selected from those listed in Table 1. The term also includes samples that have been manipulated in any way after their procurement, such as by treatment with reagents, solubilization, or enrichment for certain components, such as proteins or polynucleotides.

As used herein, the term “peripheral biological fluid sample” typically refers to a biological fluid sample that is not derived from the central nervous system (i.e., is not a CSF sample) and includes blood samples and other biological fluids not derived from the CNS (e.g., tears, saliva, urine).

The terms “AD biomarker” and the like, when used herein, are not intended to indicate the biomarker is only to be used to aid in the diagnosis, diagnose, monitor or stratify an individual with AD. As this disclosure makes clear, the biomarkers of the present invention are also useful for, for example, assessing cognitive function, assessing MCI, stratifying AD, etc., as well as assessing cognitive function and stratifying other neurological disorders, such as those associated with neurodegeneration.

The term “biomarker polynucleotide”, as used herein, typically refers to any of: a polynucleotide sequence encoding a biomarker of the present invention, the associated trans-acting control elements (e.g., promoter, enhancer, and other gene regulatory sequences), and/or mRNA encoding a biomarker of the present invention.

As used herein, methods for “aiding diagnosis” typically refer to methods that assist in making a clinical determination regarding the presence, or nature, of the neurological disorder (such as AD) and may or may not be conclusive with respect to the definitive diagnosis. Accordingly, for example, a method of aiding diagnosis of neurological disorder can comprise measuring the amount of one or more biomarkers, as herein described, in a biological sample from an individual. In another example, a method of aiding diagnosis of a neurological condition according to the present invention can be used in combination with other methods of clinical assessment of a neurological disorder, including, but not limited to, memory and/or psychological tests, imaging examination (such as magnetic resonance imaging (MRI) and positron emission tomography (PET)), assessment of language impairment and/pr other focal cognitive deficits (such as apraxia, acalculia and left-right disorientation), assessment of impaired judgment and general problem-solving difficulties, assessment of personality changes ranging from progressive passivity to marked agitation and measuring the level of amyloid beta and tau proteins in the cerebral spinal fluid of a patient.

As used herein, the term “stratifying” typically refers to sorting individuals into different classes or strata based on the features of a neurological disease. For example, stratifying a population of individuals with a neurological disorder involves assigning the individuals on the basis of the severity of the disease (e.g., mild, moderate, advanced, etc.).

As used herein, the terms “neurological disease” and “neurological disorder” typically refer to a disease or disorder of the central nervous system. Neurological diseases or disorders include, but are not limited to multiple sclerosis, neuropathies, and neurodegenerative disorders such as AD, Parkinson's disease, amyotrophic lateral sclerosis (ALS), mild cognitive impairment (MCI), Downs's and all forms of dementia including front temporal dementia, Dementia with Lewy Bodies, Vascular dementia, Parkinson's disease dementia etc. The term includes all diseases that are similar and linked to AD/MCI. Approximately 30% of Parkinson's disease patients develop Alzheimer's disease. Iron homeostasis and oxidative stress stress plays a role in both diseases as well as inflammatory pathway. People with Downs's syndrome develop amyloid plaque pathology and many go on to develop Alzheimer's disease. Optionally the neurological diseases or disorders include those with specific inflammatory and amyloid plaque forming pathology.

As used herein, the term “individual” typically refers to a mammal and includes, but is not limited to, humans, primates, farm animals, rodents and pets.

A “normal” individual or a sample from a “normal” individual, as used herein for quantitative and qualitative data, typically refers to an individual who has or would be assessed by a physician as not having AD or other neurological condition and has an Mini-Mental State Examination (MMSE) score or would achieve a MMSE score in the range of 25-30 (see Folstein et al., J. Psychiatr. Res 1975; 12:1289-198). A “Normal” individual is typically age-matched within a range of 5 to 10 years, including, but not limited to, an individual that is age-matched with the individual to be assessed.

As used herein, an “individual with mild AD” is typically an individual who (i) has been diagnosed with AD or has been given a diagnosis of probable AD, and (ii) has either been assessed with the Mini-Mental State Examination (MMSE) and scored 22-27 or would achieve a score of 22-27 upon MMSE testing. Accordingly, “mild AD”, as used herein, typically refers to AD in an individual who has either been assessed with the MMSE and scored 22-27 or would achieve a score of 22-27 upon MMSE testing.

As used herein, an “individual with moderate AD” is an individual who (i) has been diagnosed with AD or has been given a diagnosis of probable AD, and (ii) has either been assessed with the MMSE and scored 16-21 or would achieve a score of 16-21 upon MMSE testing. Accordingly, “moderate AD”, as used herein, refers to AD in an individual who has either been assessed with the MMSE and scored 16-21 or would achieve a score of 16-21 upon MMSE testing.

As used herein, an “individual with severe AD” is an individual who (i) has been diagnosed with AD or has been given a diagnosis of probable AD, and (ii) has both been assessed with the MMSE and scored 12-15 or would achieve a score of 12-15 upon MMSE testing. Accordingly, “severe AD”, as used herein, refers to AD in an individual who has both been assessed with the MMSE and scored 12-15 or would achieve a score of 12-15 upon MMSE testing.

As used herein, the term “treatment” typically refers to the alleviation, amelioration, and/or stabilization of symptoms, as well as delay in progression of symptoms of a particular disorder. For example, “treatment” of AD includes any one or more of: elimination of one or more symptoms of AD, reduction of one or more symptoms of AD, stabilization of the symptoms of AD (e.g., failure to progress to more advanced stages of AD), and delay in progression (i.e., worsening) of one or more symptoms of AD.

As used herein, the term “fold difference” typically refers to a numerical representation of the magnitude difference between a measured value and a reference value for a biomarker of the present invention. Fold difference can be calculated mathematically by division of the numeric measured value with the numeric reference value. For example, if a measured value for a biomarker is 20 nanograms/milliliter (ng/ml) and the reference value is 10 ng/ml, the fold difference is 2. Alternatively, if a measured value for a biomarker is 10 nanograms/milliliter (ng/ml), and the reference value is 20 ng/ml, the fold difference is −0.50 (or −50%).

As used herein, a “reference value” can be an absolute value, a relative value, a value that has an upper and/or lower limit, a range of values, an average value, a median value, a mean value or a value as compared to a particular control or baseline value. A reference value can be based on an individual sample value, such as, for example, a value obtained from a sample from an individual with AD, MCI or cognitive impairment, but at an earlier point in time, or a value obtained from a sample from an AD patient other than the individual being tested, or a “normal” individual, as hereinbefore described (i.e., an individual not diagnosed with AD or other neurological condition). The reference value can be based on a large number of samples, such as from AD patients or normal individuals or based on a pool of samples including or excluding the sample to be tested. A reference value may be derived from a sample taken at an earlier point in time.

As used herein, “a”, “an”, and “the” can mean singular or plural (i.e., can mean one or more) unless indicated otherwise.

As used herein, the term “naturally-occurring” typically refers to a peptide biomarker (or a variant thereof) having an amino acid sequence that occurs in nature (e.g., a natural protein). Where the biomarker is a polynucleotide, it would be understood by the skilled addressee that the term “naturally-occurring” typically refers to a biomarker having a nucleotide sequence that occurs in nature.

As used herein, a “variant” of a biomarker may exhibit an amino acid or nucleic acid sequence that is at least 80% identical to a native molecule. Also encompassed by the term “variant” are naturally-occurring molecules that have an amino acid or nucleic acid sequence that is at least 90% identical, preferably at least 95% identical, more preferably at least 98% identical, even more preferably at least 99% identical, or most preferably at least 99.9% identical to the native molecule. Percent identity may be determined by visual inspection and mathematical calculation. Among the naturally-occurring variants provided are variants of a native biomarker that retain native biological activity or a substantial equivalent thereof. Also provided herein are naturally-occurring variants that have no substantial biological activity, such as those derived from mutations or a precursor of a biologically active biomarker.

Variants of the biomarkers of the present invention may also include polypeptides or polynucleotides that are substantially homologous to the native form of the biomarker, but which have an amino acid or nucleic acid sequence that is different from that of the native form because of one or more deletions, insertions or substitutions. In some embodiments, variants include polypeptides or polynucleotides that comprise from one to ten deletions, insertions or substitutions of amino acid or nucleic acid residues when compared to the native form. A given sequence may be replaced, for example, by a residue having similar physiochemical characteristics. Examples of such conservative substitution of one aliphatic residue for another, such as Ile, Val, Leu or Ala for one another; substitution of one polar residue for another, such as between Lys and Arg, Glu and Asp, or Gln and Asn; or substitutions of one aromatic residue for another, such as Phe, Trp or Tyr for one another. Other conservative substitutions, for example, involving substitutions of entire regions having similar hydrophobicity characteristics, are well known in the art. Variants may also be generated by the truncation of a native molecule. A “conservative amino acid substitution” is typically one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, an amino acid residue of a biomarker polypeptide may be replaced with another amino acid residue from the same side chain family.

The biological activity of a biomarker can be assessed by the skilled addressee by any number of means known in the art depending upon the nature of the biomarker in question.

Assessment of results derived by the methods of the present invention can depend on whether the data were obtained by the qualitative or quantitative methods described herein and/or type of reference point used. For example, as described herein in the Examples, quantitative or absolute values (e.g., protein concentration levels) in a biological fluid sample may be obtained. “Quantitative” results or data typically refer to absolute values that can include a concentration of a biomarker in pg/ml or ng/ml in a sample. An example of a quantitative value is the measurement of concentration of protein levels directly for example by ELISA. “Qualitative” result or data typically refers to a relative value which is compared to a reference value.

The results may also be assessed and compared by one or more of the statistical methods selected from the group consisting of Random Forest, Support Vector Machine, Linear Models for MicroArray data (LIMMA) and/or Significance Analyses of Microarray Data (SAM), Best First, Greedy Stepwise, Naive Bayes, Linear Forward Selection, Scatter Search, Linear Discriminant Analysis (LDA), Stepwise Logistic Regression, Receiver Operating Characteristic and Classification Trees (CT).

In some embodiments, multiple reagents specific for the biomarkers of the present invention are attached to a suitable substrate (surface), for example, as slide, filter or beads. Qualitative assessment of results may include normalizing data. In this disclosure, various sets of biomarkers are described. It is understood that the invention contemplates use of any of these sets, any one or more members of the sets, as well as markers comprising the sets.

Reagents may include antibodies or antigen-binding fragments thereof (including, for example, polyclonal, monoclonal, humanized, anti-idiotypic, chimeric or single chain antibodies, and FAb, F(ab′)₂ and FAb expression library fragments, scFV molecules, and epitope-binding fragments thereof), oligonucleotides or fragments or other small molecules that are capable of binding to a biomarker of the present invention.

Methods of Assessing, Diagnosing or aiding in the Diagnosis of Cognitive Impairment

The present invention also provides a method of assessing cognitive function, assessing cognitive impairment, diagnosing or aiding diagnosis of cognitive impairment, the method comprising comparing a measured level of at least four biomarkers in a biological sample from an individual to a reference level for the at least four biomarkers, wherein the at least four biomarkers are selected from a panel of markers consisting of:

| | | |
|---|---|---|
| Cortisol | SOD—superoxide | MPO—Myeloperoxidase |
| | dismutase | |
| IGF.BP.2—insulin-like | TIMP-1—tissue inhibitor of | Neut—neutrophils |
| growth factor binding | metalloproteinase 1 | |
| protein 2 | | |
| IL.17—interleukin-17 | Adiponectin | PCV—packed cell volume |
| Pancreatic Polypeptide | BLC—chemokine (C—X—C | Rb85—Rubidium |
| | motif) ligand | |
| ApoE ECU— | β2 Microglobin | RCC—red cell count |
| apolipoprotein E | | |
| Calcium Corrected (Ca corr = | Cancer Antigen 19.9 | rFol—red cell folate |
| Ca total + ((40 − alb) * | | |
| 0.02)) | | |
| ABeta 42 | Eotaxin | Selenium |
| Apolipoprotein E4 Allelle | MIP-1-α—chemokine (C-C | TNF.RII—Tumor necrosis |
| | motif) ligand 3 | factor receptor superfamily |
| | | member 1B |
| VCAM-1—vascular cell | alb/tpr | tPr (total protein) |
| adhesion molecule 1 | | |
| Alb—albumin | CD40—CD40 molecule | VEGF Vascular endothelial |
| | | growth factor |
| B2M—beta-2-microglobulin | Chromium isotope | ANG-2—Angiopoietin-2 |
| | 52/Chromium isotope 53 | |
| CEA—carcinoembryonic | FT3 | α-2-macroglobulin |
| antigen | | |
| EGF.R—epidermal growth | HCY—homocysteine | EGFR—Epidermal growth |
| factor receptor | | factor receptor |
| Hb—haemoglobin | IL.10—interleukin 10 | Hepatocyte Growth Factor |
| | | (HGF) |
| Zinc | MCHC—mean cell | ICAM-1—Intercellular |
| | haemoglobin concentration | adhesion molecule 1 |
| Triiodothyronine | MMP.2—matrix | TNF receptor superfamily |
| | metallopeptidase 2 (72 kDa | member 5 |
| | type IV collagenase | |

and naturally-occurring variants thereof.

In another embodiment, at least two of the at least four biomarkers are selected from the group consisting of:
Cortisol
IGF.BP.2—insulin-like growth factor binding protein 2
IL.17—interleukin—17
Pancreatic Polypeptide
ApoE ECU—apolipoprotein E
Calcium Corrected (Ca corr=Ca total+((40−alb)*0.02))
ABeta 42
Apolipoprotein E4 Allelle
VCAM-1—vascular cell adhesion molecule 1
   and naturally-occurring variants thereof.

In some embodiments, comparing the measured level of the at least four biomarkers in the biological sample from the individual comprises comparing the measured level of at least three, four, five, six, seven, eight or nine biomarkers, selected from the panel of markers consisting of:
Cortisol
IGF.BP.2—insulin-like growth factor binding protein 2
IL.17—interleukin—17
Pancreatic Polypeptide
ApoE ECU—apolipoprotein E
Calcium Corrected (Ca corr=Ca total+((40−alb)*0.02))
ABeta 42
Apolipoprotein E4 Allelle
VCAM−1—vascular cell adhesion molecule 1
   and naturally-occurring variants thereof.

It would be understood by the skilled addressee that the degree of sensitivity and/or selectivity of the methods of the present invention in aiding diagnosis, diagnosing and/or monitoring an individual with a neurological disorder and/or stratifying an individual, as herein described, will generally be greater where the method comprises comparing the measured level of nine biomarkers in the biological sample.

In some embodiments, the methods of the present invention further comprises comparing a measured level of at least one other biomarker in a biological sample from the individual in combination with a measured level of the at least four biomarkers to a reference level for the at least one other biomarker and the at least four biomarkers, wherein the at least one other biomarker is selected from apanel of markers consisting of:

| | |
|---|---|
| Alb—albumin | SOD—superoxide |
| | dismutase |
| B2M—beta-2-microglobulin | TIMP-1—tissue inhibitor of |
| | metalloproteinase 1 |
| CEA—carcinoembryonic | Adiponectin |
| antigen | |
| EGF.R—epidermal growth | BLC—chemokine (C—X—C |
| factor receptor | motif) ligand |
| Hb—haemoglobin | β2 Microglobin |
| Zinc | Cancer Antigen 19.9 |
| IL.17—interleukin-17 | Eotaxin |
| VCAM-1—vascular cell | MIP-1-α—chemokine (C-C |
| adhesion molecule 1 | motif) ligand 3 |

and naturally-occurring variants thereof.

In some embodiments, comparing the measured level of the at least one other biomarker in a biological sample from the individual comprises comparing the measured level of at least up to all of the biomarkers, selected from the group consisting of:

| | |
|---|---|
| Alb—albumin | SOD—superoxide |
| | dismutase |
| B2M—beta-2-microglobulin | TIMP-1—tissue inhibitor of |
| | metalloproteinase 1 |
| CEA—carcinoembryonic | Adiponectin |
| antigen | |
| EGF.R—epidermal growth | BLC—chemokine (C—X—C |
| factor receptor | motif) ligand |
| Hb—haemoglobin | β2 Microglobin |
| Zinc | Cancer Antigen 19.9 |
| IL.17—interleukin-17 | Eotaxin |
| VCAM-1—vascular cell | MIP-1-α—chemokine (C-C |
| adhesion molecule 1 | motif) ligand 3 |

and naturally-occurring variants thereof.

It would be understood by the skilled addressee that the degree of sensitivity and/or selectivity of the methods of the present invention in aiding diagnosis, diagnosing and/or monitoring an individual with a neurological disorder and/or stratifying an individual, as herein described, will generally be greater where the method comprises comparing the measured level of all nine other biomarkers in the biological sample, although it may suffice compare the measured level of one other biomarker in the biological sample from the individual.

In some embodiments, the methods of the present invention further comprise comparing a measured level of at least another biomarker marker in a biological sample from the individual in combination with a measured level of the at least four biomarkers to a reference level for the at least another biomarker and the at least four biomarkers, wherein the at least another biomarker is selected from a tertiary panel of markers consisting of:

| | |
|---|---|
| alb/tpr | MPO—Myeloperoxidase |
| CD40—CD40 molecule | Neut—neutrophils |
| Chromium isotope | PCV—packed cell volume |
| 52/Chromium isotope 53 | |
| FT3 | Rb85—Rubidium |
| HCY—homocysteine | RCC—red cell count |
| IL.10—interleukin 10 | rFol—red cell folate |
| MCHC—mean cell | Selenium |
| haemoglobin concentration | |
| MMP.2—matrix | TNF.RII—Tumor necrosis |
| metallopeptidase 2 (72 kDa | factor receptor superfamily |
| type IV collagenase | member 1B |
| EGFR—Epidermal growth | tPr (total protein) |
| factor receptor | |
| Hepatocyte Growth Factor | VEGF Vascular endothelial |
| (HGF) | growth factor |
| ICAM-1—Intercellular | ANG-2—Angiopoietin-2 |
| adhesion molecule 1 | |
| TNF receptor superfamily | α-2-macroglobulin |
| member 5 | |
| Triiodothyronine | |

and naturally-occurring variants thereof.

In some embodiments, comparing the measured level of the at least another biomarker in a biological sample from the individual comprises comparing the measured level of at least up to all of the biomarkers, selected from the group consisting of:

| | |
|---|---|
| alb/tpr | MPO—Myeloperoxidase |
| CD40—CD40 molecule | Neut—neutrophils |
| Chromium isotope | PCV—packed cell volume |
| 52/Chromium isotope 53 | |
| FT3 | Rb85—Rubidium |
| HCY—homocysteine | RCC—red cell count |
| IL.10—interleukin 10 | rFol—red cell folate |
| MCHC—mean cell | Selenium |
| haemoglobin concentration | |
| MMP.2—matrix | TNF.RII—Tumor necrosis |
| metallopeptidase 2 (72 kDa | factor receptor superfamily |
| type IV collagenase | member 1B |
| EGFR—Epidermal growth | tPr (total protein) |
| factor receptor | |
| Hepatocyte Growth Factor | VEGF Vascular endothelial |
| (HGF) | growth factor |
| ICAM-1—Intercellular | ANG-2—Angiopoietin-2 |
| adhesion molecule 1 | |
| TNF receptor superfamily | α-2-macroglobulin |
| member 5 | |
| Triiodothyronine | |

and naturally-occurring variants thereof.

It would be understood by the skilled addressee that the degree of sensitivity and/or selectivity of the methods of the present invention in aiding diagnosis, diagnosing and/or monitoring an individual with a neurological disorder and/or stratifying an individual, as herein described, will generally be greater where the method comprises comparing the measured level of all eleven tertiary biomarkers in the biological sample, although it may suffice compare the measured level of another biomarker in the biological sample from the individual.

In some embodiments, the method of the present invention comprises comparing the measured level of the at four biomarkers in a biological sample from the individual and further comprising comparing the measured level of at least one other biomarker in a biological sample from the individual, whether the measured levels of the at least four biomarkers and the at least one other biomarker are from the same biological sample or from different biological samples from the individual. In some embodiments, the method of the present invention comprises comparing the measured level of the at least four biomarkers in a biological sample from the individual and further comprising comparing the measured level of at least another biomarker in a biological sample from the individual, whether the measured levels of the at least four biomarkers and the at least another biomarker are from the same biological sample or from different biological samples from the individual. In some embodiments, the method of the present invention comprises comparing the measured level of the at least four biomarkers in a biological sample from the individual and further comprising comparing the measured level of the at least one one other biomarker in a biological sample from the individual and further comprising comparing the measured level of the at least another biomarker in a biological sample from the individual, whether the measured levels of the at least four biomarkers, the at least one other biomarker and the at least another biomarker are from the same biological sample or from different biological samples from the individual.

It would also be understood by the skilled addressee that, in some embodiments of the present invention, the degree of sensitivity and/or selectivity of the methods of the present invention in aiding diagnosis, diagnosing and/or monitoring an individual with a neurological disorder and/or stratifying an individual, as herein described, may be greater where the method comprises comparing the measured level of all biomarkers in the biological sample with the at least one other biomarker and/or the at least another biomarker, as herein described.

In some embodiments of the present invention, the method of the present invention comprises comparing measured level of:
Cortisol or a naturally-occurring variant thereof
IGF.BP.2—insulin-like growth factor binding protein 2 or a naturally-occurring variant thereof
IL.17—interleukin—17 or a naturally-occurring variant thereof
Pancreatic Polypeptide or a naturally-occurring variant thereof
ApoE ECU—apolipoprotein E or a naturally-occurring variant thereof
Calcium Corrected (Ca corr=Ca total+((40−alb)*0.02)) or a naturally-occurring variant thereof
ABeta 42 or a naturally-occurring variant thereof
Apolipoprotein E4 Allelle
VCAM−1—vascular cell adhesion molecule 1 or a naturally-occurring variant thereof
   in the biological sample of the individual to reference levels for the biomarkers.

### Method of Stratifying an Individual

In yet another aspect of the present invention, there is provided a method of stratifying an individual (i.e., sorting an individual with a probable diagnosis of a neurological disorder or diagnosed with a neurological disorder into different classes of the disorder) into one or more classes of a neurological disorder, the method comprising comparing a measured level of at least four biomarkers in a biological sample from an individual to a reference level for the at least four biomarkers, wherein the at least four biomarkers are selected from a panel of markers consisting of:

| | | |
|---|---|---|
| Cortisol | SOD—superoxide | MPO—Myeloperoxidase |
| | dismutase | |
| IGF.BP.2—insulin-like | TIMP-1—tissue inhibitor of | Neut—neutrophils |
| growth factor binding | metalloproteinase 1 | |
| protein 2 | | |
| IL.17—interleukin-17 | Adiponectin | PCV—packed cell volume |
| Pancreatic Polypeptide | BLC—chemokine (C—X—C | Rb85—Rubidium |
| | motif) ligand | |
| ApoE ECU— | β2 Microglobin | RCC—red cell count |
| apolipoprotein E | | |
| Calcium Corrected (Ca corr = | Cancer Antigen 19.9 | rFol—red cell folate |
| Ca total + ((40 − alb) * | | |
| 0.02)) | | |
| ABeta 42 | Eotaxin | Selenium |
| Apolipoprotein E4 Allelle | MIP-1-α—chemokine (C-C | TNF.RII—Tumor necrosis |
| | motif) ligand 3 | factor receptor superfamily |
| | | member 1B |
| VCAM-1—vascular cell | alb/tpr | tPr (total protein) |
| adhesion molecule 1 | | |
| Alb—albumin | CD40—CD40 molecule | VEGF Vascular endothelial |
| | | growth factor |
| B2M—beta-2-microglobulin | Chromium isotope | ANG-2—Angiopoietin-2 |
| | 52/Chromium isotope 53 | |
| CEA—carcinoembryonic | FT3 | α-2-macroglobulin |
| antigen | | |
| EGF.R—epidermal growth | HCY—homocysteine | EGFR—Epidermal growth |
| factor receptor | | factor receptor |
| Hb—haemoglobin | IL.10—interleukin 10 | Hepatocyte Growth Factor |
| | | (HGF) |
| Zinc | MCHC—mean cell | ICAM-1—Intercellular |
| | haemoglobin concentration | adhesion molecule 1 |
| Triiodothyronine | MMP.2—matrix | TNF receptor superfamily |
| | metallopeptidase 2 (72 kDa | member 5 |
| | type IV collagenase | |

and naturally-occurring variants thereof.

In another embodiment, at least two of the at least four biomarkers are selected from the group consisting of:
Cortisol
IGF.BP.2—insulin-like growth factor binding protein 2
IL.17—interleukin—17
Pancreatic Polypeptide
ApoE ECU—apolipoprotein E
Calcium Corrected (Ca corr=Ca total+((40−alb)*0.02))
ABeta 42
Apolipoprotein E4 Allelle
VCAM-1—vascular cell adhesion molecule 1
   and naturally-occurring variants thereof.

In some embodiments, comparing the measured level of the at least four biomarkers in the biological sample from the individual comprises comparing the measured level of at least three three, four, five, six, seven, eight or nine biomarkers selected from the group consisting of:
Cortisol
IGF.BP.2—insulin-like growth factor binding protein 2
IL.17—interleukin—17
Pancreatic Polypeptide
ApoE ECU—apolipoprotein E
Calcium Corrected (Ca corr=Ca total+((40−alb)*0.02))
ABeta 42
Apolipoprotein E4 Allelle
VCAM-1—vascular cell adhesion molecule 1

It would be understood by the skilled addressee that the degree of sensitivity and/or selectivity of the methods of the present invention in aiding diagnosis, diagnosing and/or monitoring an individual with a neurological disorder and/or stratifying an individual, as herein described, will generally be greater where the method comprises comparing the measured level of up to all biomarkers in the biological sample.

In some embodiments, the methods of the present invention further comprises comparing a measured level of at least one other biomarker in a biological sample from the individual in combination with a measured level of the at least four biomarkers to a reference level for the at least one other biomarker and the at least four biomarkers, wherein the at least one other biomarker is selected from a panel of markers consisting of:

| | |
|---|---|
| Alb—albumin | SOD—superoxide |
| | dismutase |
| B2M—beta-2-microglobulin | TIMP-1—tissue inhibitor of |
| | metalloproteinase 1 |
| CEA—carcinoembryonic | Adiponectin |
| antigen | |
| EGF.R—epidermal growth | BLC—chemokine (C—X—C |
| factor receptor | motif) ligand |
| Hb—haemoglobin | β2 Microglobin |
| Zinc | Cancer Antigen 19.9 |
| IL.17—interleukin-17 | Eotaxin |
| VCAM-1—vascular cell | MIP-1-α—chemokine (C-C |
| adhesion molecule 1 | motif) ligand 3 |

and naturally-occurring variants thereof.

In some embodiments, comparing the measured level of the at least one other biomarker in a biological sample from the individual comprises comparing the measured level of at least up to sixteen other biomarkers. It would be understood by the skilled addressee that the degree of sensitivity and/or selectivity of the methods of the present invention in aiding diagnosis, diagnosing and/or monitoring an individual with a neurological disorder and/or stratifying an individual, as herein described, will generally be greater where the method comprises comparing the measured level of all nine other biomarkers in the biological sample, although it may suffice compare the measured level of one other biomarker in the biological sample from the individual.

In some embodiments, the methods of the present invention further comprises comparing a measured level of at least another biomarker marker in a biological sample from the individual in combination with a measured level of the at least four biomarkers to a reference level for the at least another biomarker and the at least four biomarkers, wherein the at least another biomarker is selected from a tertiary panel of markers consisting of:

| | |
|---|---|
| alb/tpr | MPO—Myeloperoxidase |
| CD40—CD40 molecule | Neut—neutrophils |
| Chromium isotope | PCV—packed cell volume |
| 52/Chromium isotope 53 | |
| FT3 | Rb85—Rubidium |
| HCY—homocysteine | RCC—red cell count |
| IL.10—interleukin 10 | rFol—red cell folate |
| MCHC—mean cell | Selenium |
| haemoglobin concentration | |
| MMP.2—matrix | TNF.RII—Tumor necrosis |
| metallopeptidase 2 (72 kDa | factor receptor superfamily |
| type IV collagenase | member 1B |
| EGFR—Epidermal growth | tPr (total protein) |
| factor receptor | |
| Hepatocyte Growth Factor | VEGF Vascular endothelial |
| (HGF) | growth factor |
| ICAM-1—Intercellular | ANG-2—Angiopoietin-2 |
| adhesion molecule 1 | |
| TNF receptor superfamily | α-2-macroglobulin |
| member 5 | |
| Triiodothyronine | |

and naturally-occurring variants thereof.

In some embodiments, comparing the measured level of the at least another biomarker in a biological sample from the individual comprises comparing the measured level of at least three to twenty five sequentially of the another biomarkers. It would be understood by the skilled addressee that the degree of sensitivity and/or selectivity of the methods of the present invention in aiding diagnosis, diagnosing and/or monitoring an individual with a neurological disorder and/or stratifying an individual, as herein described, will generally be greater where the method comprises comparing the measured level of all eleven tertiary biomarkers in the biological sample, although it may suffice compare the measured level of another biomarker in the biological sample from the individual.

In some embodiments, the method of the present invention comprises comparing the measured level of the four biomarkers in a biological sample from the individual and further comprising comparing the measured level of the at least one other biomarker in a biological sample from the individual, whether the measured level of the biomarkers are from the same biological sample or from different biological samples from the individual. In some embodiments, the method of the present invention comprises comparing the measured level of the four biomarkers in a biological sample from the individual and further comprising comparing the measured level of the at least another biomarker in a biological sample from the individual, whether the measured level of the biomarkers are from the same biological sample or from different biological samples from the individual. In some embodiments, the method of the present invention comprises comparing the measured level of the four biomarkers in a biological sample from the individual and further comprising comparing the measured level of the at least one other biomarker in a biological sample from the individual and further comprising comparing the measured level of the at least another biomarker in a biological sample from the individual, whether the measured level of the biomarkers are from the same biological sample or from different biological samples from the individual.

In some embodiments of the present invention, the method of the present invention comprises comparing measured level of:
Cortisol or a naturally-occurring variant thereof
IGF.BP.2—insulin-like growth factor binding protein 2 or a naturally-occurring variant thereof
IL.17—interleukin—17 or a naturally-occurring variant thereof
Pancreatic Polypeptide or a naturally-occurring variant thereof
ApoE ECU—apolipoprotein E or a naturally-occurring variant thereof
Calcium Corrected (Ca corr=Ca total+((40−alb)*0.02)) or a naturally-occurring variant thereof
ABeta 42 or a naturally-occurring variant thereof
Apolipoprotein E4 Allelle
VCAM−1—vascular cell adhesion molecule 1 or a naturally-occurring variant thereof
   in the biological sample of the individual to reference levels for the biomarkers.

### Methods of Monitoring the Progression of a Neurological Disorder

In a further aspect of the present invention, there is provided a method of monitoring progression of a neurological disorder, the method comprising comparing a measured level of at least four biomarkers in a biological sample from an individual to a reference level for the at least four biomarkers, wherein the at least four biomarkers are selected from a panel of markers consisting of:

| | | |
|---|---|---|
| Cortisol | SOD—superoxide | MPO—Myeloperoxidase |
| | dismutase | |
| IGF.BP.2—insulin-like | TIMP-1—tissue inhibitor of | Neut—neutrophils |
| growth factor binding | metalloproteinase 1 | |
| protein 2 | | |
| IL.17—interleukin-17 | Adiponectin | PCV—packed cell volume |
| Pancreatic Polypeptide | BLC—chemokine (C—X—C | Rb85—Rubidium |
| | motif) ligand | |
| ApoE ECU— | β2 Microglobin | RCC—red cell count |
| apolipoprotein E | | |
| Calcium Corrected (Ca corr = | Cancer Antigen 19.9 | rFol—red cell folate |
| Ca total + ((40 − alb) * | | |
| 0.02)) | | |
| ABeta 42 | Eotaxin | Selenium |
| Apolipoprotein E4 Allelle | MIP-1-α—chemokine (C-C | TNF.RII—Tumor necrosis |
| | motif) ligand 3 | factor receptor superfamily |
| | | member 1B |
| VCAM-1—vascular cell | alb/tpr | tPr (total protein) |
| adhesion molecule 1 | | |
| Alb—albumin | CD40—CD40 molecule | VEGF Vascular endothelial |
| | | growth factor |
| B2M—beta-2-microglobulin | Chromium isotope | ANG-2—Angiopoietin-2 |
| | 52/Chromium isotope 53 | |
| CEA—carcinoembryonic | FT3 | α-2-macroglobulin |
| antigen | | |
| EGF.R—epidermal growth | HCY—homocysteine | EGFR—Epidermal growth |
| factor receptor | | factor receptor |
| Hb—haemoglobin | IL.10—interleukin 10 | Hepatocyte Growth Factor |
| | | (HGF) |
| Zinc | MCHC—mean cell | ICAM-1—Intercellular |
| | haemoglobin concentration | adhesion molecule 1 |
| Triiodothyronine | MMP.2—matrix | TNF receptor superfamily |
| | metallopeptidase 2 (72 kDa | member 5 |
| | type IV collagenase | |

and naturally-occurring variants thereof.

In another embodiment, at least two of the at least four biomarkers are selected from the group consisting of:
Cortisol
IGF.BP.2—insulin-like growth factor binding protein 2
IL.17—interleukin—17
Pancreatic Polypeptide
ApoE ECU—apolipoprotein E
Calcium Corrected (Ca corr=Ca total+((40−alb)*0.02))
ABeta 42
Apolipoprotein E4 Allelle
VCAM-1—vascular cell adhesion molecule 1
   and naturally-occurring variants thereof.

In some embodiments, comparing the measured level of the at least four biomarkers in the biological sample from the individual comprises comparing the measured level of at least up to nine (numbered sequentially) biomarkers for the panel comprising:
Cortisol
IGF.BP.2—insulin-like growth factor binding protein 2
IL.17—interleukin—17
Pancreatic Polypeptide
ApoE ECU—apolipoprotein E
Calcium Corrected (Ca corr=Ca total+((40−alb)*0.02))
ABeta 42
Apolipoprotein E4 Allelle
VCAM−1—vascular cell adhesion molecule 1
   and naturally-occurring variants thereof.

It would be understood by the skilled addressee that the degree of sensitivity and/or selectivity of the methods of the present invention in aiding diagnosis, diagnosing and/or monitoring an individual with a neurological disorder and/or stratifying an individual, as herein described, will generally be greater where the method comprises comparing the measured level of all biomarkers in the biological sample.

In some embodiments, the methods of the present invention further comprise comparing a measured level of at least one other biomarker in a biological sample from the individual in combination with a measured level of the at least four biomarkers to a reference level for the at least one other biomarker and the at least four biomarkers, wherein the at least one other biomarker is selected from a panel of markers consisting of:

| | |
|---|---|
| Alb—albumin | SOD—superoxide |
| | dismutase |
| B2M—beta-2-microglobulin | TIMP-1—tissue inhibitor of |
| | metalloproteinase 1 |
| CEA—carcinoembryonic | Adiponectin |
| antigen | |
| EGF.R—epidermal growth | BLC—chemokine (C—X—C |
| factor receptor | motif) ligand |
| Hb—haemoglobin | β2 Microglobin |
| Zinc | Cancer Antigen 19.9 |
| IL.17—interleukin - 17 | Eotaxin |
| VCAM-1—vascular cell | MIP-1-α—chemokine (C-C |
| adhesion molecule 1 | motif) ligand 3 |

and naturally-occurring variants thereof.

In some embodiments, comparing the measured level of the at least one other biomarker in a biological sample from the individual comprises comparing the measured level of at least up to sixteen of the biomarkers, It would be understood by the skilled addressee that the degree of sensitivity and/or selectivity of the methods of the present invention in aiding diagnosis, diagnosing and/or monitoring an individual with a neurological disorder and/or stratifying an individual, as herein described, will generally be greater where the method comprises comparing the measured level of all nine other biomarkers in the biological sample, although it may suffice compare the measured level of one other biomarker in the biological sample from the individual.

In some embodiments, the methods of the present invention further comprise comparing a measured level of at least another biomarker marker in a biological sample from the individual in combination with a measured level of the at least four biomarkers to a reference level for the at least another biomarker and the at least four biomarkers, wherein the at least another biomarker is selected from a panel of markers consisting of:

| | |
|---|---|
| alb/tpr | MPO—Myeloperoxidase |
| CD40—CD40 molecule | Neut—neutrophils |
| Chromium isotope | PCV—packed cell volume |
| 52/Chromium isotope 53 | |
| FT3 | Rb85—Rubidium |
| HCY—homocysteine | RCC—red cell count |
| IL.10—interleukin 10 | rFol—red cell folate |
| MCHC—mean cell | Selenium |
| haemoglobin concentration | |
| MMP.2—matrix | TNF.RII—Tumor necrosis |
| metallopeptidase 2 (72 kDa | factor receptor superfamily |
| type IV collagenase | member 1B |
| EGFR—Epidermal growth | tPr (total protein) |
| factor receptor | |
| Hepatocyte Growth Factor | VEGF Vascular endothelial |
| (HGF) | growth factor |
| ICAM-1—Intercellular | ANG-2—Angiopoietin-2 |
| adhesion molecule 1 | |
| TNF receptor superfamily | α-2-macroglobulin |
| member 5 | |
| Triiodothyronine | |

and naturally-occurring variants thereof.

In some embodiments, comparing the measured level of the at least another biomarker in a biological sample from the individual comprises comparing the measured level of at least up to all of the another biomarkers. It would be understood by the skilled addressee that the degree of sensitivity of the methods of the present invention in aiding diagnosis, diagnosing and/or monitoring an individual with a neurological disorder and/or stratifying an individual, as herein described, will generally be greater where the method comprises comparing the measured level of all eleven tertiary biomarkers in the biological sample, although it may suffice compare the measured level of another biomarker in the biological sample from the individual.

In some embodiments, the method of the present invention comprises comparing the measured level of the at four biomarkers in a biological sample from the individual and further comprising comparing the measured level of the at least one other biomarker in a biological sample from the individual, whether the measured level of the biomarkers are from the same biological sample or from different biological samples from the individual. In some embodiments, the method of the present invention comprises comparing the measured level of the four biomarkers in a biological sample from the individual and further comprising comparing the measured level of the at least another biomarker in a biological sample from the individual, whether the measured level of the biomarkers are from the same biological sample or from different biological samples from the individual. In some embodiments, the method of the present invention comprises comparing the measured level of the at four biomarkers in a biological sample from the individual and further comprising comparing the measured level of the at least one one other biomarker in a biological sample from the individual and further comprising comparing the measured level of the at least another biomarker in a biological sample from the individual, whether the measured level of the biomarkers are from the same biological sample or from different biological samples from the individual.

In some embodiments of the present invention, the method of the present invention comprises comparing measured level of:
Cortisol or a naturally-occurring variant thereof
IGF.BP.2—insulin-like growth factor binding protein 2 or a naturally-occurring variant thereof
IL.17—interleukin—17 or a naturally-occurring variant thereof
Pancreatic Polypeptide or a naturally-occurring variant thereof
ApoE ECU—apolipoprotein E or a naturally-occurring variant thereof
Calcium Corrected (Ca corr=Ca total+((40−alb)*0.02)) or a naturally-occurring variant thereof
ABeta 42 or a naturally-occurring variant thereof
Apolipoprotein E4 Allelle
VCAM−1—vascular cell adhesion molecule 1 or a naturally-occurring variant thereof
   in the biological sample of the individual to reference levels for the biomarkers.

In some embodiments of the present invention, measured levels for the biomarkers are obtained from an individual at more than one time point. Such “serial” sampling is well suited, for example, to monitoring the progression of the neurological disorder. Serial sampling can be performed on any desired timeline, such as monthly, quarterly (i.e., every three months), semi-annually, annually, biennially, or less frequently. The comparison between the measured levels and the reference level may be carried out each time a new sample is measured, or the data relating to levels may be held for less frequent analysis.

In some embodiments of the present invention, biological samples including peripheral biological fluid samples are collected from individuals who are suspected of having a neurological disorder or developing a neurological disorder such as AD or MCI. The present invention also contemplates samples from individuals for whom cognitive assessment is desired. Alternatively, individuals (or others involved in, for example, research and/or clinicians) may desire such assessments without any indication of a neurological disorder or suspected neurological disorder. For example, a normal individual may desire such information. In some embodiments, individuals are 65 years or older, although individuals from whom biological samples, such as peripheral biological fluid samples are taken for use in the methods of the present invention may be as young as 35 to 40 years old, when early onset AD or familial AD is suspected.

### Methods for Identifying Biomarkers

The present invention also provides a method for identifying one or more biomarkers useful for diagnosis, aiding in diagnosis and/or monitoring a neurological disorder and/or stratifying an individual (i.e., sorting an individual with a probable diagnosis of a neurological disorder or diagnosed with a neurological disorder into different classes of the disorder).

In one aspect of the present invention, there is provided a method of identifying at least one biomarker for use in diagnosing, aiding diagnosis and/or monitoring progression of a neurological disorder in an individual and/or stratifying an individual, the method comprising obtaining measured values from a set of biological samples for a plurality of biomarkers, wherein the set of biological samples is divisible into subsets on the basis of a neurological disorder, comparing the measured values from each subset for at least one biomarker; and identifying at least one biomarker for which the measured values are significantly different between the subsets.

In some embodiments, comparing the measured values from each subset for at least one biomarker is carried out by one or more of the statistical methods selected from the group consisting of Random Forest, Support Vector Machine, Linear Models for MicroArray data (LIMMA) and/or Significance Analyses of Microarray Data (SAM), Best First, Greedy Stepwise, Naive Bayes, Linear Forward Selection, Scatter Search, Linear Discriminant Analysis (LDA), Stepwise Logistic Regression, Receiver Operating Characteristic and Classification Trees (CT).

In some embodiments, the method comprises comparing the measured values from each subset for at least one biomarker by using Boosted Trees (BT). In some embodiments, the method provides sensitivity of at least 85% and specificity of at least 85% in diagnosing or aiding diagnosis of a neurological disorder in an individual.

In some embodiments, the method comprises comparing the measured values from each subset for at least one biomarker is carried out by a combination of Random Forest, Support Vector Machine, Linear Models for MicroArray data (LIMMA) and/or Significance Analyses of Microarray Data (SAM), Best First, Greedy Stepwise, Naive Bayes, Linear Forward Selection, Scatter Search, Linear Discriminant Analysis (LDA), Stepwise Logistic Regression and Receiver Operating Characteristic and Classification trees.

In some embodiments, the at least one biomarker is selected from the group consisting of:

| | | |
|---|---|---|
| Cortisol | SOD—superoxide | MPO—Myeloperoxidase |
| | dismutase | |
| IGF.BP.2—insulin-like | TIMP-1—tissue inhibitor of | Neut—neutrophils |
| growth factor binding | metalloproteinase 1 | |
| protein 2 | | |
| IL.17—interleukin - 17 | Adiponectin | PCV—packed cell volume |
| Pancreatic Polypeptide | BLC—chemokine (C—X—C | Rb85—Rubidium |
| | motif) ligand | |
| ApoE ECU— | β2 Microglobin | RCC—red cell count |
| apolipoprotein E | | |
| Calcium Corrected (Ca corr = | Cancer Antigen 19.9 | rFol—red cell folate |
| Ca total + ((40 − alb) * | | |
| 0.02)) | | |
| ABeta 42 | Eotaxin | Selenium |
| Apolipoprotein E4 Allelle | MIP-1-α—chemokine (C-C | TNF.RII—Tumor necrosis |
| | motif) ligand 3 | factor receptor superfamily |
| | | member 1B |
| VCAM-1—vascular cell | alb/tpr | tPr (total protein) |
| adhesion molecule 1 | | |
| Alb—albumin | CD40—CD40 molecule | VEGF Vascular endothelial |
| | | growth factor |
| B2M—beta-2-microglobulin | Chromium isotope | ANG-2—Angiopoietin-2 |
| | 52/Chromium isotope 53 | |
| CEA—carcinoembryonic | FT3 | α-2-macroglobulin |
| antigen | | |
| EGF.R—epidermal growth | HCY—homocysteine | EGFR—Epidermal growth |
| factor receptor | | factor receptor |
| Hb—haemoglobin | IL.10—interleukin 10 | Hepatocyte Growth Factor |
| | | (HGF) |
| Zinc | MCHC—mean cell | ICAM-1—Intercellular |
| | haemoglobin concentration | adhesion molecule 1 |
| Triiodothyronine | MMP.2—matrix | TNF receptor superfamily |
| | metallopeptidase 2 (72 kDa | member 5 |
| | type IV collagenase | |

and naturally-occurring variants thereof.

In some embodiments, the method comprises comparing the measured values from each subset for the at least one biomarker and may further include comparing the age of individuals from which the set of biological samples was obtained, as herein described (see, e.g., Examples herein).

In some embodiments of the present invention, levels of a panel of biomarkers are obtained for a set of biological samples from one or more individuals. The samples are selected such that they can be segregated into one or more subsets on the basis of a neurological disease (e.g., samples from normal individuals and those diagnosed with amyotrophic lateral sclerosis or samples from individuals with mild AD and those with severe AD and/or other neurological diseases, such as neurodegenerative diseases). The measured values from the samples are compared to each other to identify those biomarkers which differ significantly amongst the subsets. Those biomarkers that vary significantly amongst the subsets may then be used in methods for aiding in the diagnosis, diagnosis, stratification and/or monitoring a neurological disorder, as herein described.

In other aspects of the present invention, measured values of a panel of biomarkers in a set of biological samples from one or more individuals (where the samples can be segregated into one or more subsets on the basis of a neurological disorder) are compared, wherein biomarkers that vary significantly are useful for aiding in the diagnosis, diagnosis, stratification and/or monitoring a neurological disease, as herein described. In further aspects of the present invention, levels (e.g., concentration, expression and/or activity) of a group of biomarkers in a set of biological fluid samples from one or more individuals (where the samples can be segregated into one or more subsets on the basis of a neurological disorder) are measured to produce measured values, wherein biomarkers whose levels vary significantly (e.g., from a reference level) are useful for aiding in the diagnosis, diagnosis, stratification and/or monitoring a neurological disorder, as herein described.

This aspect of the present invention typically utilizes a set of biological samples, such as blood samples, that are derived from one or more individuals. The set of samples is selected such that it can be divided into one or more subsets on the basis of a neurological disorder or severity of a neurological disorder. The division into subsets can be on the basis of presence/absence of disease, stratification of disease (e.g., mild vs. moderate), or subclassification of disease (e.g., relapsing/remitting vs. progressive relapsing).

Biomarkers measured in the practice of the present invention may be, for example, any proteinaceous biological marker found in a biological sample of a subject. Table 1 includes a collection or panel of exemplary biomarkers.

Accordingly, in one aspect of the present invention, there is provided a method of identifying at least one biomarker which can be used to aid in the diagnosis, to diagnose, detect and/or stratify a neurological disorder, as herein described. In some embodiments, the methods of the present invention are carried out by obtaining a set of measured values for a plurality of biomarkers from a set of biological samples, where the set of biological samples is divisible into at least two subsets in relation to a neurological disorder, comparing said measured values between the subsets for each biomarker, and identifying biomarkers which are significantly different between the subsets.

The process of comparing the measured values may be carried out by any method known in the art, including Significance Analysis of Microarrays, Tree Harvesting, CART, MARS, Self Organizing Maps, Frequent Item Set, or Bayesian networks. In some embodiments of the present invention, the process of comparing the measured values is carried out by one or more of the statistical methods selected from the group consisting of Random Forest (RF), Boosted Trees (BT), Linear Models for Micro Array data (LIMMA), Classification Trees (CT), Linear Discriminant Analysis (LDA), Stepwise Logistic Regression and Receiver Operating Characteristic (ROC).

RF (classification) is a variable selection method that uses classification trees to infer class membership to each case. RF grows a number of classification trees (a forest), and counts the number of votes from trees (each tree provides a vote for a specific class) to predict class membership. RF outputs a variable importance, which is a relative measure on how well each variable is able to predict the class membership. Variable importance is plotted as the mean decrease in accuracy from each RF model. To compile a reduced list of useful biomarkers, and increase the accuracy of class prediction, multiple RF iterations after variable reduction, based upon variable importance, can be computed.

The LIMMA method has been widely used in the analysis of micro array data. Its general purpose to identify gene expression difference between two classes where P>>N (i.e., more variables than observations). The method typically starts with fitting a standard linear model to the data, and then uses an Empirical Bayes approach to borrow information across variables (reduction of sample error), and uses a moderated t-statistic with an augmented degrees of freedom. The LIMMA method outputs a False Discovery Rate (FDR) adjusted p-value (the q-value) which is useful in the relative difference between mean samples. The LIMMA method can be used to determine differences in mean biomarker level between HC and AD participants.

The CT method is an alternative approach to a non-linear regression where there are many complex interactions between multiple variables, whether they are continuous or categorical in nature. The method creates multiple partitions or subdivisions of data (recursive partitioning) so that the interaction between multiple variables becomes simpler. Recursive partitioning is analogous to creating multiple classification trees, where the interior branches are questions, and the outer leaves are the answers to the questions. Once simple partitions or trees have been formulated, simple local models are computed before outputting final tree structures, including criterions at which each branch (or variable) should be split by. This method has advantages in that (i) it allows one to see what variables are selected for the final tree in the model, and (ii) it allows for further biomarker analyses in combination with Receiver Operating Characteristic (ROC) analyses; integrating lifestyle, genetic markers and biomarkers to identify proportional AD risk.

BT (classification) is a variable selection and class prediction method that builds an initial binary classification tree (a root node and two child nodes), and then fits another tree based upon the partition residuals from the prior tree. This computation can be iterated many times, and acts as a weighted remodelling process prior to votes for class prediction are totaled from all trees. BT outputs a relative influence measure that, similar to the variable importance, provides a relative measure on how well each variable is able to predict class membership. The BT method also produces a predicted probability of class membership, which is useful for comparison of predicted class membership to actual class membership.

LDA is a statistical method that determines a linear combination of variables that separate two or more class groups.

Stepwise Logistic Regression is a statistical method whereby many predictor variables are added to a Logistic Regression framework, and multiple “steps” are taken to add/remove variables to decrease the error within the statistical model. In this way, the method accurately assesses each of the variables added into the model and determines how much each contributes to the prediction. Thus, using the biomarkers (including age) chosen from the RF, BT, LIMMA and CT methods as herein described, the Stepwise Logistic Regression can be performed and compared with the standard Logistic Regression.

The ROC method has been primarily used as a diagnostic tool to define a criterion by which a certain markers can correctly classify a person into a designated class. ROC analyses provides multiple outcomes, one of which, the Area Under the Curve (AUC) is a useful measure for assessing model performance. The AUC statistic can be utilized within the biomarker analysis to compare Logistic Regression and Stepwise Logistic Regression models (e.g., from training set data) using different numbers of biomarkers. Sensitivity and specificity from statistical models computed on test set data can also be plotted to provide a graphical comparison of the performance of the models.

In one aspect, the present invention provides a method for identifying at least one biomarker useful for diagnosing, aiding diagnosis of a neurological disorder in an individual and/or monitoring progression of a neurological disorder in an individual and/or stratifying a patient (i.e., sorting an individual with a probable diagnosis of a neurological disorder or diagnosed with a neurological disorder into different classes of the disorder), the method comprising obtaining measured values from a set of biological samples for a plurality of biomarkers, wherein the set of biological samples is divisible into subsets on the basis of a neurological disorder or severity of a neurological disorder, comparing the measured values from each subset for at least one biomarker; and identifying at least one biomarker for which the measured values are different (e.g., significantly different) between the subsets. In some embodiments, the comparing process is carried out using Significance Analysis of Microarrays. In some embodiments, the neurological disorder is Alzheimer's disease.

In some embodiments, the at least one biomarker is selected from the group consisting of:

| | | |
|---|---|---|
| Cortisol | SOD—superoxide | MPO—Myeloperoxidase |
| | dismutase | |
| IGF.BP.2—insulin-like | TIMP-1—tissue inhibitor of | Neut—neutrophils |
| growth factor binding | metalloproteinase 1 | |
| protein 2 | | |
| IL.17—interleukin - 17 | Adiponectin | PCV—packed cell volume |
| Pancreatic Polypeptide | BLC—chemokine (C—X—C | Rb85—Rubidium |
| | motif) ligand | |
| ApoE ECU— | β2 Microglobin | RCC—red cell count |
| apolipoprotein E | | |
| Calcium Corrected (Ca corr = | Cancer Antigen 19.9 | rFol—red cell folate |
| Ca total + ((40 − alb) * | | |
| 0.02)) | | |
| ABeta 42 | Eotaxin | Selenium |
| Apolipoprotein E4 Allelle | MIP-1-α—chemokine (C-C | TNF.RII—Tumor necrosis |
| | motif) ligand 3 | factor receptor superfamily |
| | | member 1B |
| VCAM-1—vascular cell | alb/tpr | tPr (total protein) |
| adhesion molecule 1 | | |
| Alb—albumin | CD40—CD40 molecule | VEGF Vascular endothelial |
| | | growth factor |
| B2M—beta-2-microglobulin | Chromium isotope | ANG-2—Angiopoietin-2 |
| | 52/Chromium isotope 53 | |
| CEA—carcinoembryonic | FT3 | α-2-macroglobulin |
| antigen | | |
| EGF.R—epidermal growth | HCY—homocysteine | EGFR—Epidermal growth |
| factor receptor | | factor receptor |
| Hb—haemoglobin | IL.10—interleukin 10 | Hepatocyte Growth Factor |
| | | (HGF) |
| Zinc | MCHC—mean cell | ICAM-1—Intercellular |
| | haemoglobin concentration | adhesion molecule 1 |
| Triiodothyronine | MMP.2—matrix | TNF receptor superfamily |
| | metallopeptidase 2 (72 kDa | member 5 |
| | type IV collagenase | |

and naturally-occurring variants thereof.

Tables 2 and 3 disclosed herein provide a listing of biomarkers (clustered by the methods as described herein) that are increased or decreased in AD subjects as compared to age-matched normal controls or other non-AD forms of neurodegeneration, such as, for example, PD and PN (that is, as compared to all controls). Any one or more of the biomarkers listed in Tables 1 to 3, or reagents specific for the biomarker, can be used in the methods disclosed herein, such as for example, for aiding in the diagnosis of or diagnosing AD or to diagnose AD as distinguished from other non-AD neurodegenerative diseases or disorders, such as for example PD and PN.

Accordingly, in some examples, positively correlated AD biomarkers for use in the methods of the present invention, as herein described, such as, for example, for aiding in the diagnosis of or diagnosing neurological disorders, including AD, are selected from the group consisting of biomarkers listed in Tables 2 and 3.

In some embodiments, the methods of the present invention can be used before, after and/or concurrently with other methods of aiding diagnosis, diagnosing and/or monitoring a neurological disorder in an individual and/or stratifying an individual, as herein described, for example, as a one other screen.

The present invention also provides methods of evaluating the results of the methods as herein described. Such evaluation generally entails reviewing the results and can assist, for example, in advising medical practitioners and others regarding clinical and/or diagnostic follow-up and/or treatment options. The present invention also provides methods for assessing a biological sample for an indicator of any one or more of the following: cognitive function and/or impairment; MCI; AD; extent of AD (e.g., mild, moderate, severe); and progression of AD by measuring the level of or obtaining the measured level of or comparing a measured level of an AD biomarker as herein described. Methods of assessing cognitive impairment may include the ADAS-COG, which is generally accepted to be equivalent to MMSE scoring.

### Methods of Assessing Efficacy of Treatment Modalities

In some embodiments, the present invention also provides methods for assessing the efficacy of treatment modalities in an individual or a population of individuals, such as from a single or multiple collection centre(s), subject to impaired cognitive function and/or diagnosed with a neurological disorder comprising (i) comparing a measured level of at least four biomarkers in a biological sample from an individual to a reference level for the at least four biomarkers, wherein the at least four biomarkers are selected from a panel of markers consisting of:

| | | |
|---|---|---|
| Cortisol | SOD—superoxide | MPO—Myeloperoxidase |
| | dismutase | |
| IGF.BP.2—insulin-like | TIMP-1—tissue inhibitor of | Neut—neutrophils |
| growth factor binding | metalloproteinase 1 | |
| protein 2 | | |
| IL.17—interleukin - 17 | Adiponectin | PCV—packed cell volume |
| Pancreatic Polypeptide | BLC—chemokine (C—X—C | Rb85—Rubidium |
| | motif) ligand | |
| ApoE ECU— | β2 Microglobin | RCC—red cell count |
| apolipoprotein E | | |
| Calcium Corrected (Ca corr = | Cancer Antigen 19.9 | rFol—red cell folate |
| Ca total + ((40 − alb) * | | |
| 0.02)) | | |
| ABeta 42 | Eotaxin | Selenium |
| Apolipoprotein E4 Allelle | MIP-1-α—chemokine (C-C | TNF.RII—Tumor necrosis |
| | motif) ligand 3 | factor receptor superfamily |
| | | member 1B |
| VCAM-1—vascular cell | alb/tpr | tPr (total protein) |
| adhesion molecule 1 | | |
| Alb—albumin | CD40—CD40 molecule | VEGF Vascular endothelial |
| | | growth factor |
| B2M—beta-2-microglobulin | Chromium isotope | ANG-2—Angiopoietin-2 |
| | 52/Chromium isotope 53 | |
| CEA—carcinoembryonic | FT3 | α-2-macroglobulin |
| antigen | | |
| EGF.R—epidermal growth | HCY—homocysteine | EGFR—Epidermal growth |
| factor receptor | | factor receptor |
| Hb—haemoglobin | IL.10—interleukin 10 | Hepatocyte Growth Factor |
| | | (HGF) |
| Zinc | MCHC—mean cell | ICAM-1—Intercellular |
| | haemoglobin concentration | adhesion molecule 1 |
| Triiodothyronine | MMP.2—matrix | TNF receptor superfamily |
| | metallopeptidase 2 (72 kDa | member 5 |
| | type IV collagenase | |

and naturally-occurring variants thereof.

In another embodiment, at least two of the at least four biomarkers are selected from the group consisting of:
Cortisol
IGF.BP.2—insulin-like growth factor binding protein 2
IL.17—interleukin—17
Pancreatic Polypeptide
ApoE ECU—apolipoprotein E
Calcium Corrected (Ca corr=Ca total+((40−alb)*0.02))
ABeta 42
Apolipoprotein E4 Allelle
VCAM-1—vascular cell adhesion molecule 1
   and naturally-occurring variants thereof.

In some embodiments, comparing the measured level of the at least four biomarkers in the biological sample from the individual comprises comparing the measured level of at least up to all biomarkers. It would be understood by the skilled addressee that the degree of sensitivity and/or selectivity of the methods of the present invention in aiding diagnosis, diagnosing and/or monitoring an individual with a neurological disorder and/or stratifying an individual, as herein described, will generally be greater where the method comprises comparing the measured level of all biomarkers listed herein in the biological sample.

Typically, diagnosing efficacy of treatment will be based on a comparison of measured levels to an appropriate reference, wherein the appropriate reference is a measured level taken before the onset of treatment and/or during treatment. Measured levels of the at least four biomarkers may be obtained once or multiple times during assessment of the treatment modality.

In some embodiments, the methods of the present invention further comprise comparing a measured level of at least one other biomarker in the biological sample from the individual in combination with a measured level of the at least four biomarkers to a reference level for the at least one other biomarker and the at least four biomarkers, wherein the at least one other biomarker is selected from a other panel of markers consisting of:

| | |
|---|---|
| Alb—albumin | SOD—superoxide |
| | dismutase |
| B2M—beta-2-microglobulin | TIMP-1—tissue inhibitor of |
| | metalloproteinase 1 |
| CEA—carcinoembryonic | Adiponectin |
| antigen | |
| EGF.R—epidermal growth | BLC—chemokine (C—X—C |
| factor receptor | motif) ligand |
| Hb—haemoglobin | β2 Microglobin |
| Zinc | Cancer Antigen 19.9 |
| IL.17—interleukin - 17 | Eotaxin |
| VCAM-1—vascular cell | MIP-1-α—chemokine (C-C |
| adhesion molecule 1 | motif) ligand 3 |

and naturally-occurring variants thereof.

In some embodiments, comparing the measured level of the at least one other biomarker in a biological sample from the individual comprises comparing the measured level of at least up to sixteen other biomarkers. It would be understood by the skilled addressee that the degree of sensitivity and/or selectivity of the methods of the present invention in aiding diagnosis, diagnosing and/or monitoring an individual with a neurological disorder and/or stratifying an individual, as herein described, will generally be greater where the method comprises comparing the measured level of all sixteen other biomarkers in the biological sample, although it may suffice to compare the measured level of one other biomarker in the biological sample from the individual.

In some embodiments, the methods of the present invention further comprise comparing a measured level of at least another biomarker marker in a biological sample from the individual in combination with a measured level of the at least four biomarkers to a reference level for the at least another biomarker and the at least four biomarkers, wherein the at least another biomarker is selected from a tertiary panel of markers consisting of:

| | |
|---|---|
| alb/tpr | MPO—Myeloperoxidase |
| CD40—CD40 molecule | Neut—neutrophils |
| Chromium isotope | PCV—packed cell volume |
| 52/Chromium isotope 53 | |
| FT3 | Rb85—Rubidium |
| HCY—homocysteine | RCC—red cell count |
| IL.10—interleukin 10 | rFol—red cell folate |
| MCHC—mean cell | Selenium |
| haemoglobin concentration | |
| MMP.2—matrix | TNF.RII—Tumor necrosis |
| metallopeptidase 2 (72 kDa | factor receptor superfamily |
| type IV collagenase | member 1B |
| EGFR—Epidermal growth | tPr (total protein) |
| factor receptor | |
| Hepatocyte Growth Factor | VEGF Vascular endothelial |
| (HGF) | growth factor |
| ICAM-1—Intercellular | ANG-2—Angiopoietin-2 |
| adhesion molecule 1 | |
| TNF receptor superfamily | α-2-macroglobulin |
| member 5 | |
| Triiodothyronine | |

and naturally-occurring variants thereof.

In some embodiments, comparing the measured level of the at least another biomarker in a biological sample from the individual comprises comparing the measured level of at least up to all of the another biomarkers. It would be understood by the skilled addressee that the degree of sensitivity and/or selectivity of the methods of the present invention in aiding diagnosis, diagnosing and/or monitoring an individual with a neurological disorder and/or stratifying an individual, as herein described, will generally be greater where the method comprises comparing the measured level of all of the another biomarkers in the biological sample, although it may suffice compare the measured level of one another biomarker in the biological sample from the individual.

In some embodiments, the methods for assessing the efficacy of treatment modalities in an individual or a population of individuals, such as from a single or multiple collection centre(s), subject to impaired cognitive function and/or diagnosed with a neurological disorder of the present invention comprises comparing the measured level of the four biomarkers in a biological sample from the individual and further comprising comparing the measured level of the at least one other biomarker in a biological sample from the individual, whether the measured level of the biomarkers are from the same biological sample or from different biological samples from the individual. In some embodiments, the method of the present invention comprises comparing the measured level of the at four biomarkers in a biological sample from the individual and further comprising comparing the measured level of the at least another biomarker in a biological sample from the individual, whether the measured level of the primary and tertiary biomarkers are from the same biological sample or from different biological samples from the individual. In some embodiments, the method of the present invention comprises comparing the measured level of the four biomarkers in a biological sample from the individual and further comprising comparing the measured level of the at least one other biomarker in a biological sample from the individual and further comprising comparing the measured level of the at least another biomarker in a biological sample from the individual, whether the measured level of the primary, one other and tertiary biomarkers are from the same biological sample or from different biological samples from the individual.

In some embodiments of the present invention, the method of the present invention comprises comparing the measured level of:
Cortisol or a naturally-occurring variant thereof
IGF.BP.2—insulin-like growth factor binding protein 2 or a naturally-occurring variant thereof
IL.17—interleukin—17 or a naturally-occurring variant thereof
Pancreatic Polypeptide or a naturally-occurring variant thereof
ApoE ECU—apolipoprotein E or a naturally-occurring variant thereof
Calcium Corrected (Ca corr=Ca total+((40−alb)*0.02)) or a naturally-occurring variant thereof
ABeta 42 or a naturally-occurring variant thereof
Apolipoprotein E4 Allelle
VCAM−1—vascular cell adhesion molecule 1 or a naturally-occurring variant thereof

It would be understood by those skilled in the art that the relative concentration of a biomarkers of the present invention, as herein described, in serum, CSF, or other biological sample, as a composite (or collective) or any subset of such a composite, composed of five or more elements is more predictive than the absolute concentration of any individual biomarker in predicting clinical phenotypes, disease detection, stratification, monitoring, and treatment of AD, PD, frontotemporal dementia, cerebrovascular disease, multiple sclerosis, and neuropathies.

Although the use of any one of the biomarkers of the present invention for practice of the methods of the present invention may provide acceptable levels of sensitivity and specificity, it would be understood by the skilled addressee that the effectiveness (e.g., sensitivity and/or specificity) of the methods of the present invention are typically enhanced when more that four biomarkers are utilized. In some embodiments of the present invention, the methods are generally enhanced when at least five biomarkers are utilized, such as those listed in Table 2 herein.

Multiple biomarkers may be selected from the biomarkers disclosed herein by a variety of methods, including Random Forest, Support Vector Machine, Linear Models for MicroArray data (LIMMA) and/or Significance Analyses of Microarray Data (SAM), Best First, Greedy Stepwise, Naive Bayes, Linear Forward Selection, Scatter Search, Linear Discriminant Analysis (LDA), Stepwise Logistic Regression and Receiver Operating Characteristic and Classification Trees. The present inventors used these methods in combination to identify a small set of biomarkers that have good sensitivity and specificity for predicting clinical phenotype.

### REFERENCE LEVELS

For methods of diagnosing a neurological disorder (such as AD), as described herein, the reference level is typically a predetermined level considered “normal” for a given biomarker (e.g., an average level for one or more age-matched individuals not diagnosed with a neurological disorder or an average level for one or more age-matched individuals diagnosed with another neurological disorder and/or healthy age-matched individuals), although reference levels which are determined contemporaneously (e.g., a reference value that is derived from a pool of samples including the sample being tested) are also contemplated by the present invention. For the biomarkers of the present invention, a measured level for a biomarker which is below or above the reference level suggests (i.e., aids in the diagnosis of) or indicates a diagnosis of a neurological disorder.

If the comparison between the measured level(s) of a biomarker and the reference level(s) indicates a difference (that is, an increase or decrease) that is suggestive/indicative of a neurological disorder (e.g., AD or MCI), then the appropriate diagnosis is aided in or made. Conversely, if the comparison of the measured level(s) to the reference level(s) does not indicate differences that suggest or indicate a diagnosis of the neurological condition, then the appropriate diagnosis is not aided in or made.

The reference level used for comparison with the measured level for a biomarker may vary, depending on the aspect of the present invention being practiced, as will be understood from the foregoing discussion. For diagnosis methods, the “reference level” is typically a predetermined reference level, such as an average of levels obtained from a population that is not afflicted with the neurological condition that is the subject of the diagnostic method (including normal, healthy individuals), but in some instances, the reference level can be a mean or median level from a group of individuals including those with a neurological disorder, such as AD. In some embodiments of the present invention, the predetermined reference level is derived from (e.g., is the mean or median of) levels obtained from an age-matched population. In some embodiments of the present invention, the age-matched population comprises individuals with non-AD neurodegenerative disorders.

For MCI diagnosis methods (i.e., methods of diagnosing or aiding in the diagnosis of MCI), the reference level may be a predetermined reference level, such as an average of levels obtained from a population that is not afflicted with AD or MCI, but in some instances, the reference level can be a mean or median level from a group of individuals including MCI and/or AD patients. In some embodiments of the present invention, the predetermined reference level is derived from (e.g., is the mean or median of) levels obtained from an age-matched population.

For AD monitoring methods (e.g., methods of diagnosing or aiding in the diagnosis of AD progression in an AD patient), the reference level may be a predetermined level, such as an average of levels obtained from a population that is not afflicted with AD or MCI, a population that has been diagnosed with MCI or AD, and, in some instances, the reference level can be a mean or median level from a group of individuals including MCI and/or AD patients. Alternately, the reference level may be a historical reference level for the particular patient (e.g., a biomarker level that was obtained from a sample derived from the same individual, but at an earlier point in time). In some instances, the predetermined reference level is derived from (e.g., is the mean or median of) levels obtained from an age-matched population.

For AD stratification methods (i.e., methods of stratifying AD patients into mild, moderate and severe stages of AD), the reference level may be a predetermined reference level that is the mean or median of levels from a population which has been diagnosed with AD or MCI (preferably a population diagnosed with AD). In some embodiments of the present invention, the predetermined reference level is derived from (e.g., is the mean or median of) levels obtained from an age-matched population.

Age-matched populations (from which reference values may be obtained) are ideally the same age as the individual being tested, but approximately age-matched populations are also acceptable. Approximately age-matched populations may be within 1, 2, 3, 4, or 5 years of the age of the individual tested, or may be groups of different ages which encompass the age of the individual being tested. Approximately age-matched populations may be in 2, 3, 4, 5, 6, 7, 8, 9, or year increments (e.g. a “5 year increment” group which serves as the source for reference values for a 62 year old individual might include 58-62 year old individuals, 59-63 year old individuals, 60-64 year old individuals, 61-65 year old individuals, or 62-66 year old individuals).

Identifying and/or Measuring Levels of Biomarkers

In some embodiments, a biomarker is considered “identified” as being useful for aiding in the diagnosis, diagnosis, stratification and/or monitoring a neurological disorder, as herein described, when it is significantly different between the subsets of peripheral biological samples tested. Levels of a biomarker are “significantly different” typically when the probability that the particular biomarker has been identified by chance is less than a predetermined value. The method of calculating such probability will depend on the exact method utilizes to compare the levels between the subsets (e.g., if SAM is used, the q-value will give the probability of misidentification, and the p value will give the probability if the t test (or similar statistical analysis) is used). As will be understood by those skilled in the art, the predetermined value will vary depending on the number of biomarkers measured per sample and the number of samples utilized. Accordingly, a predetermined value may range from as high as 50% to as low as 20%, 10%, 5%, 3%, 2%, or 1%.

As herein described, the level of the at least four biomarkers is measured in one or more biological samples from an individual. The biomarker levels may be measured using any available measurement technology that is capable of measuring the level of the biomarkers in a biological sample. The measurement may be either quantitative or qualitative, so long as the measurement is capable of indicating whether the level of the biomarkers in the peripheral biological fluid sample is above or below a reference value.

The measured level may be a primary measurement of the level a particular biomarker, a measurement of the quantity of biomarker itself (quantitative data, such as by detecting the number of biomarker molecules in the sample) or it may be a secondary measurement of the biomarker (a measurement from which the quantity of the biomarker can be but not necessarily deduced (qualitative data), such as a measure of enzymatic activity (when the biomarker is an enzyme) or a measure of mRNA coding for the biomarker). Qualitative data may also be derived or obtained from primary measurements.

Although some assay formats will allow testing of biological samples without prior processing of the sample, it is expected that most biological samples will be processed prior to testing. Processing generally takes the form of elimination of cells (nucleated and non-nucleated), such as erythrocytes, leukocytes, and platelets in blood samples, and may also include the elimination of certain proteins, such as certain clotting cascade proteins from blood. In some examples, the peripheral biological fluid sample is collected in a container comprising EDTA.

In some embodiments, biomarker levels will be measured using an affinity-based measurement technology. As used herein, the term “affinity”, as used herein, is understood in the art and typically means the extent, or strength, of binding of an agent (e.g., an antibody, or a fragment thereof, to a biomarker, or epitope thereof), as described herein. Affinity may be measured and/or expressed in a number of ways known in the art, including, but not limited to, equilibrium dissociation constant (K_{D} or K_{d}), apparent equilibrium dissociation constant (K_{D′} or K_{d′}), and IC₅₀ (amount needed to effect 50% inhibition in a competition assay; used interchangeably herein with “I₅₀”). It would be understood to those skilled in the art that, for the purposes of the present invention, an affinity is an average affinity for a given population of antibodies which bind to an epitope. Values of K_{D′} reported herein in terms of mg IgG per ml (or mg/ml) indicate mg immunoglobulin per ml of serum, plasma or other biological sample.

Affinity-based measurement technology typically utilizes an agent that specifically binds to the biomarkers being measured (i.e., an “affinity reagent” such as an antibody, an aptamer, or a fragment thereof, as herein described), although other technologies, such as spectroscopy-based technologies (e.g., matrix-assisted laser desorption ionization-time of flight, or MALDI-TOF, spectroscopy) or assays measuring bioactivity (e.g., assays measuring mitogenicity of growth factors) may be used.

Affinity-based technologies include antibody-based assays (immunoassays) and assays utilizing aptamers (nucleic acid molecules which specifically bind to other molecules), such as ELONA. Additionally, assays utilizing both antibodies and aptamers are also contemplated (e.g., a sandwich format assay utilizing an antibody for capture and an aptamer for detection).

If immunoassay technology is employed, any immunoassay technology which can quantitatively or qualitatively measure the level of a biomarker in a biological sample may be used. Suitable immunoassay technology includes, but is not limited to, radioimmunoassay, immunofluorescent assay, enzyme immunoassay, chemiluminescent assay, enzyme-linked immunosorbant assay (ELISA), immuno-PCR, and western blot assay, multi-analyte profiling (MAP) to measure multiple proteins in small sample volumes (±100 ìL) for multiple species and sample types and is certified according to the Clinical Laboratory Improvement Amendments (CLIA)

Likewise, aptamer-based assays which can quantitatively or qualitatively measure the level of a biomarker in a biological sample may be used in the methods of the present invention. Typically, aptamers may be substituted for antibodies in nearly all formats of immunoassay, although aptamers allow additional assay formats (such as amplification of bound aptamers using nucleic acid amplification technology such as PCR (see, for example, U.S. Pat. No. 4,683,202) or isothermal amplification with composite primers (see, for example, U.S. Pat. Nos. 6,251,639 and 6,692,918).

A wide variety of affinity-based assays are known in the art. Affinity-based assays will typically utilize at least one epitope derived from the biomarker of interest, and many affinity-based assay formats utilize more than one epitope (e.g., two or more epitopes are involved in “sandwich” format assays; at least one epitope is used to capture the marker, and at least one different epitope is used to detect the marker).

Affinity-based assays may be in competition or direct reaction formats, utilize sandwich-type formats, and may further be heterogeneous (e.g., utilize solid supports) or homogenous (e.g., take place in a single phase) and/or utilize or immunoprecipitation. Many assays involve the use of a labelled affinity reagent (e.g., antibody, polypeptide, or aptamer). The labels may be, for example, enzymatic, fluorescent, chemiluminescent, radioactive, or dye molecules. Assays which amplify the signals from the probe are also known, examples of which are assays which utilize biotin and avidin, and enzyme-labelled and mediated immunoassays, such as ELISA and ELONA assays. Herein, in the examples referred to as “quantitative data”, the biomarker concentrations were obtained using ELISA. Either of the biomarker or reagent specific for the biomarker can be attached to a surface and levels can be measured directly or indirectly.

In a heterogeneous format, the assay utilizes two phases (typically aqueous liquid and solid). Typically, a biomarker-specific affinity reagent is bound to a solid support to facilitate separation of the biomarker from the bulk of the biological sample. After reaction for a time sufficient to allow for formation of affinity reagent/biomarker complexes, the solid support or surface containing the antibody (or fragment thereof) is typically washed prior to detection of bound polypeptides. The affinity reagent in the assay for measurement of a biomarker may be provided on a support (e.g., solid or semi-solid). Alternatively, the polypeptides in the sample can be immobilized on a support or surface. Examples of supports that can be used are nitrocellulose (e.g., in membrane or microtiter well form), polyvinyl chloride (e.g., in sheets or microtiter wells), polystyrene latex (e.g., in beads or microtiter plates), polyvinylidine fluoride, diazotized paper, nylon membranes, activated beads, glass and Protein A beads. Both standard and competitive formats for these assays are known in the art. Accordingly, provided herein are complexes comprising at least one biomarker bound to a reagent specific for the biomarker, wherein said reagent is attached to a surface. Also provided herein are complexes comprising at least one biomarker bound to a reagent specific for the biomarker, wherein said biomarker is attached to a surface.

Array-type heterogeneous assays are suitable for measuring levels of biomarkers when the methods of the invention are practiced utilizing multiple biomarkers. Array-type assays used in the practice of the methods of the invention will commonly utilize a solid substrate with two or more capture reagents specific for different biomarkers bound to the substrate a predetermined pattern (e.g., a grid). The peripheral biological fluid sample is applied to the substrate and biomarkers in the sample are bound by the capture reagents. After removal of the sample (and appropriate washing), the bound biomarkers are detected using a mixture of appropriate detection reagents that specifically bind the various biomarkers. Binding of the detection reagent is commonly accomplished using a visual system, such as a fluorescent dye-based system. Because the capture reagents are arranged on the substrate in a predetermined pattern, array-type assays provide the advantage of detection of multiple biomarkers without the need for a multiplexed detection system.

In a homogeneous format, the assay takes place in single phase (e.g., aqueous liquid phase). Typically, the biological sample is incubated with an affinity reagent specific for the biomarker in solution. For example, it may be under conditions that will precipitate any affinity reagent/antibody complexes which are formed. Both standard and competitive formats for these assays are known in the art.

In a standard (direct reaction) format, the level of biomarker/affinity reagent complex is directly monitored. This may be accomplished by, for example, determining the amount of a labelled detection reagent that forms is bound to biomarker/affinity reagent complexes. In a competitive format, the amount of biomarker in the sample is deduced by monitoring the competitive effect on the binding of a known amount of labelled biomarker (or other competing ligand) in the complex. Amounts of binding or complex formation can be determined either qualitatively or quantitatively.

In some embodiments of the present invention, the reagents specific for the at least one biomarker is an antibody, or a fragment thereof. Suitable antibodies are polyclonal or monoclonal antibodies. A polyclonal antibody may be produced by a method well known in the art, which includes injecting the biomarker antigen into an animal, and collecting blood samples from the animal to obtain serum containing antibodies. Such polyclonal antibodies may be prepared from any animal host, such as goats, rabbits, sheep, monkeys, horses, pigs, cows and dogs.

A reagent may be specific for (e.g., capable of binding to) more than one biomarker. For example, where the reagent is a polyclonal antibody, or mixture thereof, there will be some antibodies specific for one biomarker and another antibody specific for another biomarker.

A monoclonal antibody may be prepared by a method well known in the art, such as a hybridoma method (see Kohler and Milstein (1976) European Journal of Immunology 6: 511-519) or a phage antibody library technique (see Clackson et al., Nature, 352: 624-628, 1991; Marks et al., J. Mol. Biol., 222 (58): 1-597, 1991). The hybridoma method may employ cells extracted from an immunologically compatible host animal, such as mice, which is injected with the biomarker of interest, as one group, and a cancer or myeloma cell line as another group. Cells of these two groups are fused with each other by a method well known in the art, such as a method using polyethylene glycol, and antibody-producing cells are proliferated by a standard tissue culture method. After a uniform cell colony is obtained by subcloning using a limited dilution technique, a hybridoma capable of producing an antibody specific for (or to) the biomarker is cultivated in large quantities in vitro or in vivo according to a standard methodology. A monoclonal antibody produced by the hybridoma may be used without purification, but is typically be used after being purified by a method well known in the art so as to obtain the best outcome. The phage antibody library method is a method in which a phage antibody library is constructed in vitro by obtaining antibody genes (single-chain fragment variable (scFv) type) for a variety of biomarkers and expressing them in the form of a fusion protein on the surfaces of phages, and a monoclonal antibody capable of binding to a biomarker of the present invention is isolated from the library.

An antibody prepared by the above methods may be isolated using gel electrophoresis, dialysis, salting out, ion exchange chromatography, affinity chromatography, etc. In addition, the antibody of the present invention may include functional fragments of antibody molecules, as well as a complete form having two full-length light chains and two full-length heavy chains. The functional fragment of antibody molecules means a fragment retaining at least an antigen-binding function, and include Fab, F(ab′)2, Fv, and the like.

Specifically, suitable methodology to measure plasma ApoE methodology may be described in Lui et al, J Alzheimers Dis. 2010; 20(4):1233-42. “Plasma amyloid-beta as a biomarker in Alzheimer's disease: the AIBL study of aging”.

The methods of the present invention, as herein described, may also be implemented using any device capable of implementing the methods. Examples of devices that may be used include but are not limited to electronic computational devices, including computers of all types. When the methods of the present invention are implemented in a computer, the computer program that may be used to configure the computer to carry out the steps of the methods may be contained in any computer readable medium capable of containing the computer program. Examples of computer readable medium that may be used include but are not limited to diskettes, CD-ROMs, DVDs, ROM, RAM, and other memory and computer storage devices. The computer program that may be used to configure the computer to carry out the steps of the methods may also be provided over an electronic network, for example, over the internet, world wide web, an intranet, or other network.

In some embodiments, the methods described herein are implemented in a system comprising a processor and a computer readable medium that includes program code means for causing the system to carry out the steps of the methods of the present invention. The processor may be any processor capable of carrying out the operations needed for implementation of the methods of the present invention. The program code typically means any code that when implemented in the system can cause the system to carry out the steps of the methods of the present invention. Examples of program code means include but are not limited to instructions to carry out the methods of the present invention written in a high level computer language such as C++, Java, or Fortran; instructions to carry out the methods described in this patent written in a low level computer language such as assembly language; or instructions to carry out the methods described in this patent in a computer executable form such as compiled and linked machine language.

Complexes formed comprising a biomarker and an affinity reagent are detected by any of a number of known techniques known in the art, depending on the format of the assay and the preference of the user. For example, unlabelled affinity reagents may be detected with DNA amplification technology (e.g., for aptamers and DNA-labelled antibodies) or labelled “secondary” antibodies which bind the affinity reagent. Alternately, the affinity reagent may be labelled, and the amount of complex may be determined directly (as for dye-(fluorescent or visible), bead-, or enzyme-labelled affinity reagent) or indirectly (as for affinity reagents “tagged” with biotin, expression tags, and the like). Herein the examples provided referred to as “qualitative data” filter based antibody arrays using chemiluminescence were used to obtain measurements for biomarkers.

As will be understood by those skilled in the art, the mode of detection of the signal will depend on the exact detection system utilized in the assay. For example, if a radiolabelled detection reagent is utilized, the signal will be measured using a technology capable of quantitating the signal from the biological sample or of comparing the signal from the biological sample with the signal from a reference sample, such as scintillation counting, autoradiography (typically combined with scanning densitometry) and the like. If a chemiluminescent detection system is used, then the signal will typically be detected using a luminometer. Methods for detecting signal from detection systems are well known in the art and need not be further described here.

When more than one biomarker is measured, the biological sample may be divided into a number of aliquots, with separate aliquots used to measure different biomarkers (although division of the biological sample into multiple aliquots to allow multiple determinations of the levels of the biomarker in a particular sample are also contemplated). Alternately, the biological sample (or an aliquot therefrom) may be tested to determine the levels of multiple biomarkers in a single reaction using an assay capable of measuring the individual levels of different biomarkers in a single assay, such as an array-type assay or assay utilizing multiplexed detection technology (e.g., an assay utilizing detection reagents labelled with different fluorescent dye markers).

It is common in the art to perform “replicate” measurements when measuring biomarkers. Replicate measurements are ordinarily obtained by splitting a sample into multiple aliquots and separately measuring the biomarker(s) in separate reactions of the same assay system. Replicate measurements are not necessary to the methods of the present invention, but some embodiments of the invention will utilize replicate testing, such as duplicate and triplicate testing.

### Comparing Levels of Biomarkers

The process of comparing a measured value and a reference value according to the present invention may be carried out in any convenient manner appropriate to the type of measured value and reference value for a biomarker. As herein described, “measuring” can be performed using quantitative or qualitative measurement techniques and the mode of comparing a measured value and a reference value can vary depending on the measurement technology employed. For example, when a qualitative calorimetric assay is used to measure biomarker levels, the levels may be compared by visually comparing the intensity of the coloured reaction product, or by comparing data from densitometric or spectrometric measurements of the coloured reaction product (e.g., comparing numerical data or graphical data, such as bar charts, derived from the measuring device). However, it is expected that the measured values used in the methods of the invention will most commonly be quantitative values (e.g., quantitative measurements of concentration, such as nanograms of biomarker per milliliter of sample, or absolute amount). In other embodiments of the present invention, measured values are qualitative. As with qualitative measurements, the comparison can be made by inspecting the numerical data, by inspecting representations of the data (e.g., inspecting graphical representations such as bar or line graphs).

The process of comparing may be manual (such as visual inspection by the practitioner of the method) or it may be automated. For example, an assay device (such as a luminometer for measuring chemiluminescent signals) may include circuitry and software enabling it to compare a measured value with a reference value for a biomarker. Alternately, a separate device (e.g., a digital computer) may be used to compare the measured value(s) and the reference value(s). Automated devices for comparison may include stored reference values for the biomarker(s) being measured, or they may compare the measured value(s) with reference values that are derived from contemporaneously measured reference samples.

In some embodiments, the methods of the present invention utilize a simple or binary comparison between the measured level(s) and the reference level(s) (e.g., the comparison between a measured level and a reference level determines whether the measured level is higher or lower than the reference level). For example, a comparison showing that the measured value for a biomarker is lower than the reference value indicates or suggests a diagnosis of a neurological disorder.

As herein described, a biomarker in a biological sample may be measured quantitatively (absolute values) or qualitatively (relative values). The respective biomarker levels for a given assessment may or may not overlap. As described herein, for some embodiments of the present invention, qualitative data indicate a given level of cognitive impairment (mild, moderate or severe), which can be measured by MMSE scores, and in other embodiments of the present invention, quantitative data indicate a given level of cognitive impairment.

As will be apparent to those skilled in the art, when replicate measurements are taken for the biomarker(s) tested, the measured value that is compared with the reference value is a value that takes into account the replicate measurements. The replicate measurements may be taken into account by using either the mean or median of the measured values as the “measured value”.

### Screening Prospective Agents for Biomarker Modulation Activity

The present invention also provides methods of screening for candidate agents for the treatment of a neurological disorder by assaying prospective candidate agents for activity in modulating the biomarkers of the present invention. Such screening assays may be performed either in vitro and/or in vivo. Candidate agents identified in the screening methods as herein described may be useful as therapeutic agents, for example, for the treatment of AD, MCI and/or other neurological disorders.

Thus, it is another aspect of the present invention to provide a method of identifying candidate agents for treatment of a neurological disorder, the method comprising assaying a prospective candidate agent for activity in modulating expression and/or activity of at least four biomarkers selected from a primary panel of markers consisting of:

| | | |
|---|---|---|
| Cortisol | SOD—superoxide | MPO—Myeloperoxidase |
| | dismutase | |
| IGF.BP.2—insulin-like | TIMP-1—tissue inhibitor of | Neut—neutrophils |
| growth factor binding | metalloproteinase 1 | |
| protein 2 | | |
| IL.17—interleukin - 17 | Adiponectin | PCV—packed cell volume |
| Pancreatic Polypeptide | BLC—chemokine (C—X—C | Rb85—Rubidium |
| | motif) ligand | |
| ApoE ECU— | β2 Microglobin | RCC—red cell count |
| apolipoprotein E | | |
| Calcium Corrected (Ca corr = | Cancer Antigen 19.9 | rFol—red cell folate |
| Ca total + ((40 − alb) * | | |
| 0.02)) | | |
| ABeta 42 | Eotaxin | Selenium |
| Apolipoprotein E4 Allelle | MIP-1-α—chemokine (C-C | TNF.RII—Tumor necrosis |
| | motif) ligand 3 | factor receptor superfamily |
| | | member 1B |
| VCAM-1—vascular cell | alb/tpr | tPr (total protein) |
| adhesion molecule 1 | | |
| Alb—albumin | CD40—CD40 molecule | VEGF Vascular endothelial |
| | | growth factor |
| B2M—beta-2-microglobulin | Chromium isotope | ANG-2—Angiopoietin-2 |
| | 52/Chromium isotope 53 | |
| CEA—carcinoembryonic | FT3 | α-2-macroglobulin |
| antigen | | |
| EGF.R—epidermal growth | HCY—homocysteine | EGFR—Epidermal growth |
| factor receptor | | factor receptor |
| Hb—haemoglobin | IL.10—interleukin 10 | Hepatocyte Growth Factor |
| | | (HGF) |
| Zinc | MCHC—mean cell | ICAM-1—Intercellular |
| | haemoglobin concentration | adhesion molecule 1 |
| Triiodothyronine | MMP.2—matrix | TNF receptor superfamily |
| | metallopeptidase 2 (72 kDa | member 5 |
| | type IV collagenase | |

and naturally-occurring variants thereof.

In another embodiment, at least two of the at least four biomarkers are selected from the group consisting of:
Cortisol
IGF.BP.2—insulin-like growth factor binding protein 2
IL.17—interleukin—17
Pancreatic Polypeptide
ApoE ECU—apolipoprotein E
Calcium Corrected (Ca corr=Ca total+((40−alb)*0.02))
ABeta 42
Apolipoprotein E4 Allelle
VCAM-1—vascular cell adhesion molecule 1
   and naturally-occurring variants thereof.

In some embodiments, assaying a prospective candidate agent for activity in modulating expression and/or activity of the at least four biomarkers comprises assaying a prospective candidate agent for activity in modulating expression and/or activity of at least three three, four, five, six, seven, eight or nine biomarkers selected from the group consisting of:
Cortisol
IGF.BP.2—insulin-like growth factor binding protein 2
IL.17—interleukin—17
Pancreatic Polypeptide
ApoE ECU—apolipoprotein E
Calcium Corrected (Ca corr=Ca total+((40−alb)*0.02))
ABeta 42
Apolipoprotein E4 Allelle
VCAM−1—vascular cell adhesion molecule 1
   and naturally-occurring variants thereof.

It would be understood by the skilled addressee that the degree of sensitivity and/or selectivity of identifying candidate agents for treatment of a neurological disorder, as herein described, will generally be greater where the method comprises assaying a prospective candidate agent for activity in modulating expression and/or activity of all biomarkers.

In some embodiments, the methods of the present invention further comprises assaying a prospective candidate agent for activity in modulating expression and/or activity of at least one other biomarker in combination with assaying a prospective candidate agent for activity in modulating expression and/or activity of at least four of the biomarkers, wherein the at least one other biomarker is selected from a panel of markers consisting of:

| | |
|---|---|
| Alb—albumin | SOD—superoxide |
| | dismutase |
| B2M—beta-2-microglobulin | TIMP-1—tissue inhibitor of |
| | metalloproteinase 1 |
| CEA—carcinoembryonic | Adiponectin |
| antigen | |
| EGF.R—epidermal growth | BLC—chemokine (C—X—C |
| factor receptor | motif) ligand |
| Hb—haemoglobin | β2 Microglobin |
| Zinc | Cancer Antigen 19.9 |
| IL.17—interleukin - 17 | Eotaxin |
| VCAM-1—vascular cell | MIP-1-α—chemokine (C-C |
| adhesion molecule 1 | motif) ligand 3 |

and naturally-occurring variants thereof.

In some embodiments, assaying a prospective candidate agent for activity in modulating expression and/or activity of at least one other biomarker comprises assaying a prospective candidate agent for activity in modulating expression and/or activity of at least up to all sixteen biomarkers. It would be understood by the skilled addressee that the degree of sensitivity and/or selectivity of identifying candidate agents for treatment of a neurological disorder, as herein described, will generally be greater where the method comprises assaying a prospective candidate agent for activity in modulating expression and/or activity of all other biomarkers, although it may suffice to assay a prospective candidate agent for activity in modulating expression and/or activity of only one other biomarker.

In some embodiments, the methods of the present invention further comprises assaying a prospective candidate agent for activity in modulating expression and/or activity of at least another biomarker marker in combination with assaying a prospective candidate agent for activity in modulating expression and/or activity of the at least four biomarkers, wherein the at least another biomarker is selected from a tertiary panel of markers consisting of:

| | |
|---|---|
| alb/tpr | MPO—Myeloperoxidase |
| CD40—CD40 molecule | Neut—neutrophils |
| Chromium isotope | PCV—packed cell volume |
| 52/Chromium isotope 53 | |
| FT3 | Rb85—Rubidium |
| HCY—homocysteine | RCC—red cell count |
| IL.10—interleukin 10 | rFol—red cell folate |
| MCHC—mean cell | Selenium |
| haemoglobin concentration | |
| MMP.2—matrix | TNF.RII—Tumor necrosis |
| metallopeptidase 2 (72 kDa | factor receptor superfamily |
| type IV collagenase | member 1B |
| EGFR—Epidermal growth | tPr (total protein) |
| factor receptor | |
| Hepatocyte Growth Factor | VEGF Vascular endothelial |
| (HGF) | growth factor |
| ICAM-1—Intercellular | ANG-2—Angiopoietin-2 |
| adhesion molecule 1 | |
| TNF receptor superfamily | α-2-macroglobulin |
| member 5 | |
| Triiodothyronine | |

and naturally-occurring variants thereof.

In some embodiments, assaying a prospective candidate agent for activity in modulating expression and/or activity of the at least another biomarker comprises assaying a prospective candidate agent for activity in modulating expression and/or activity of at least up to all of the another biomarkers, It would be understood by the skilled addressee that the degree of sensitivity and/or selectivity of identifying candidate agents for treatment of a neurological disorder, as herein described, will generally be greater where the method comprises assaying a prospective candidate agent for activity in modulating expression and/or activity of all of the another biomarkers, although it may suffice to assay a prospective candidate agent for activity in modulating expression and/or activity of only another biomarker.

In some embodiments, the method of the present invention comprises assaying a prospective candidate agent for activity in modulating expression and/or activity of at least four biomarkers and further comprising assaying a prospective candidate agent for activity in modulating expression and/or activity of at least one other biomarker. In some embodiments, the method of the present invention comprises assaying a prospective candidate agent for activity in modulating expression and/or activity of at least four biomarkers and further comprising assaying a prospective candidate agent for activity in modulating expression and/or activity of at least another biomarker. In some embodiments, the method of the present invention comprises assaying a prospective candidate agent for activity in modulating expression and/or activity of at least four biomarkers and further comprising assaying a prospective candidate agent for activity in modulating expression and/or activity of at least one other biomarker and further comprising assaying a prospective candidate agent for activity in modulating expression and/or activity of at least another biomarker.

In some embodiments of the present invention, the method of the present invention comprises assaying a prospective candidate agent for activity in modulating expression and/or activity of:
Cortisol or a naturally-occurring variant thereof
IGF.BP.2—insulin-like growth factor binding protein 2 or a naturally-occurring variant thereof
IL.17—interleukin—17 or a naturally-occurring variant thereof
Pancreatic Polypeptide or a naturally-occurring variant thereof
ApoE ECU—apolipoprotein E or a naturally-occurring variant thereof
Calcium Corrected (Ca corr=Ca total+((40−alb)*0.02)) or a naturally-occurring variant thereof
ABeta 42 or a naturally-occurring variant thereof
Apolipoprotein E4 Allelle
VCAM−1—vascular cell adhesion molecule 1 or a naturally-occurring variant thereof

The screening methods of the present invention may utilize the biomarkers described herein and/or biomarker polynucleotides as “drug targets”. Prospective agents can be tested for activity in modulating a drug target in an assay system. As will be understood by those skilled in the art, the mode of testing for modulation activity will depend on the biomarker and the form of the drug target used (e.g., protein or gene). A wide variety of suitable assays are known in the art.

When the biomarker protein itself is the drug target, prospective agents are tested for activity in modulating levels or activity of the protein itself. Modulation of levels of a biomarker can be accomplished by, for example, increasing or reducing half-life of the biomarker protein. Modulation of activity of a biomarker can be accomplished by increasing or reducing the availability of the biomarker to bind to its cognate receptor(s) or ligand(s).

When a biomarker polynucleotide is the drug target, the prospective agent is tested for activity in modulating synthesis of the biomarker. The exact mode of testing for modulatory activity of a prospective agent may depend on the form of the biomarker polynucleotide selected for testing. For example, if the drug target is a biomarker polynucleotide, modulatory activity is typically tested by measuring either mRNA transcribed from the gene (transcriptional modulation) or by measuring protein produced as a consequence of such transcription (translational modulation). As will be understood by those in the art, many assay formats will utilize a modified form of the biomarker gene where a heterologous sequence (e.g., encoding an expression marker such as an enzyme or an expression tag such as oligo-histidine or a sequence derived from another protein, such as myc) is fused to (or even replaces) the sequence encoding the biomarker protein. Such heterologous sequence(s) allow for convenient detection of levels of protein transcribed from the drug target.

Prospective agents for use in the screening methods of the present invention may be chemical compounds and/or complexes of any sort, including both organic and inorganic molecules (and complexes thereof). As will be understood in the art, organic molecules are most commonly screened for biomarker modulatory activity. In some embodiments of the present invention, the prospective agents for testing will exclude the target biomarker protein.

Screening assays may be in any format known in the art, including cell-free in vitro assays, cell culture assays, organ culture assays, and in vivo assays (e.g., assays utilizing animal models of a neurological disorder, such as AD or MCI). Accordingly, the present invention also provides a variety of embodiments for screening prospective agents to identify candidate agents for the treatment of a neurological disorder.

In some embodiments of the present invention, prospective agents are screened to identify candidate agents for the treatment of a neurological disorder in a cell-free assay. Each prospective agent is incubated with the drug target in a cell-free environment and modulation of the biomarker is measured. Cell-free environments useful in the screening methods of the invention include cell lysates (particularly useful when the drug target is a biomarker gene) and biological fluids such as whole blood or fractionated fluids derived therefrom such as plasma and serum (particularly useful when the biomarker protein is the drug target). When the drug target is a biomarker gene (or polynucleotide), the modulation measured may be modulation of transcription or translation. When the drug target is the biomarker protein, the modulation may of the half-life of the protein or of the availability of the biomarker protein to bind to its cognate receptor or ligand.

In some embodiments of the present invention, prospective agents are screened to identify candidate agents for the treatment of a neurological disorder in a cell-based assay. Each prospective agent is incubated with cultured cells, and modulation of a target biomarker is measured. In certain embodiments, the cultured cells are astrocytes, neuronal cells (such as hippocampal neurons), fibroblasts, or glial cells. When the drug target is a biomarker gene (polynucleotide), transcriptional or translational modulation may be measured. When the drug target is a biomarker protein, the biomarker protein is also added to the assay mixture, and modulation of the half-life of the protein or of the availability of the biomarker protein to bind to its cognate receptor or ligand is measured.

Further embodiments of the present invention relate to screening prospective agents to identify candidate agents for the treatment of a neurological disorder in organ culture-based assays. In some embodiments, each prospective agent is incubated with either a whole organ or a portion of an organ (such as a portion of brain tissue, such as a brain slice) derived from a non-human animal and modulation of the target biomarker is measured. When the drug target is a biomarker gene (polynucleotide), transcriptional or translational modulation may be measured. When the drug target is a biomarker protein, the biomarker protein is also added to the assay mixture, and modulation of the half-life of the protein or of the availability of the biomarker protein to bind to its cognate receptor is measured.

Additional embodiments relate to screening prospective agents to identify candidate agents for the treatment of a neurological disorder utilizing in vivo assays. In some embodiments, each prospective agent is administered to a non-human animal and modulation of the target biomarker is measured. Depending on the particular drug target and the aspect of the treatment of the neurological disorder that is sought to be addressed, the animal used in such assays may either be a “normal” animal (e.g., C57 mouse) or an animal which is a model of the neurological disorder. For instance, a number of animal models of AD are known in the art, including the 3×Tg-AD mouse (Caccamo et al., 2003, Neuron 39(3):409-21), mice over expressing human amyloid beta precursor protein (APP) and presenilin genes (Westaway et al., 1997, Nat. Med. 3(1):67-72), and others (see Higgins et al., 2003, Behav. Pharmacol. 14(5-6):419-38). When the drug target is a biomarker gene (polynucleotide), transcriptional or translational modulation may be measured. When the drug target is a biomarker protein, modulation of the half-life of the target biomarker or of the availability of the biomarker protein to bind to its cognate receptor or ligand is measured. The exact mode of measuring modulation of the target AD biomarker may depend on the identity of the biomarker, the format of the assay, and the preference of the practitioner. A wide variety of methods are known in the art for measuring modulation of transcription, translation, protein half-life, protein availability, and other aspects which can be measured. In view of the common knowledge of these techniques, they need not be further described herein.

### Kits and Reagents

The present invention also provides a kit for use in the methods of the present invention, as herein described (for example, diagnosing, aiding diagnosis and/or monitoring progression of a neurological disorder in an individual and/or stratifying (i.e., sorting an individual with a probable diagnosis of a neurological disorder or diagnosed with a neurological disorder into different classes of the disorder) an individual), the kit comprising at least one reagent specific for at least four biomarkers, wherein the at least four biomarkers are selected from a primary panel of markers consisting of:

| | | |
|---|---|---|
| Cortisol | SOD—superoxide | MPO—Myeloperoxidase |
| | dismutase | |
| IGF.BP.2—insulin-like | TIMP-1—tissue inhibitor of | Neut—neutrophils |
| growth factor binding | metalloproteinase 1 | |
| protein 2 | | |
| IL.17—interleukin-17 | Adiponectin | PCV—packed cell volume |
| Pancreatic Polypeptide | BLC—chemokine (C—X—C | Rb85—Rubidium |
| | motif) ligand | |
| ApoE ECU—apolipoprotein | β2 Microglobin | RCC—red cell count |
| E | | |
| Calcium Corrected (Ca corr = | Cancer Antigen 19.9 | rFol—red cell folate |
| Ca total + ((40 − alb) * | | |
| 0.02)) | | |
| ABeta 42 | Eotaxin | Selenium |
| Apolipoprotein E4 Allelle | MIP-1-α—chemokine (C-C | TNF.RII—Tumor necrosis |
| | motif) ligand 3 | factor receptor superfamily |
| | | member 1B |
| VCAM-1—vascular cell | alb/tpr | tPr (total protein) |
| adhesion molecule 1 | | |
| Alb—albumin | CD40—CD40 molecule | VEGF Vascular endothelial |
| | | growth factor |
| B2M—beta-2-microglobulin | Chromium isotope | ANG-2—Angiopoietin-2 |
| | 52/Chromium isotope 53 | |
| CEA—carcinoembryonic | FT3 | α-2-macroglobulin |
| antigen | | |
| EGF.R—epidermal growth | HCY—homocysteine | EGFR—Epidermal growth |
| factor receptor | | factor receptor |
| Hb—haemoglobin | IL.10—interleukin 10 | Hepatocyte Growth Factor |
| | | (HGF) |
| Zinc | MCHC—mean cell | ICAM-1—Intercellular |
| | haemoglobin concentration | adhesion molecule 1 |
| Triiodothyronine | MMP.2—matrix | TNF receptor superfamily |
| | metallopeptidase 2 (72 kDa | member 5 |
| | type IV collagenase | |

and naturally-occurring variants thereof.

In another embodiment, at least two of the at least four biomarkers are selected from the group consisting of:
Cortisol
IGF.BP.2—insulin-like growth factor binding protein 2
IL.17—interleukin—17
Pancreatic Polypeptide
ApoE ECU—apolipoprotein E
Calcium Corrected (Ca corr=Ca total+((40−alb)*0.02))
ABeta 42
Apolipoprotein E4 Allelle
VCAM-1—vascular cell adhesion molecule 1
   and naturally-occurring variants thereof.

In some embodiments, the kit further comprises at least one reagent specific for at least one other biomarker in combination with the one reagent for the at least four of the biomarkers, wherein the at least one other biomarker is selected from a other panel of markers consisting of:

| | |
|---|---|
| Alb—albumin | SOD—superoxide |
| | dismutase |
| B2M—beta-2-microglobulin | TIMP-1—tissue inhibitor of |
| | metalloproteinase 1 |
| CEA—carcinoembryonic | Adiponectin |
| antigen | |
| EGF.R—epidermal growth | BLC—chemokine (C-X-C |
| factor receptor | motif) ligand |
| Hb—haemoglobin | β2 Microglobin |
| Zinc | Cancer Antigen 19.9 |
| IL.17—interleukin - 17 | Eotaxin |
| VCAM-1—vascular cell | MIP-1-α—chemokine (C-C |
| adhesion molecule 1 | motif) ligand 3 |

and naturally-occurring variants thereof.

In some embodiments, the kit further comprises at least one reagent specific for at least another biomarker in combination with the one reagent for the at least two of the primary biomarkers, wherein the at least another biomarker is selected from a tertiary panel of markers consisting of:

| | |
|---|---|
| alb/tpr | MPO—Myeloperoxidase |
| CD40—CD40 molecule | Neut—neutrophils |
| Chromium isotope | PCV—packed cell volume |
| 52/Chromium isotope 53 | |
| FT3 | Rb85—Rubidium |
| HCY—homocysteine | RCC—red cell count |
| IL.10—interleukin 10 | rFol—red cell folate |
| MCHC—mean cell | Selenium |
| haemoglobin concentration | |
| MMP.2—matrix | TNF.RII—Tumor necrosis |
| metallopeptidase 2 (72 kDa | factor receptor superfamily |
| type IV collagenase | member 1B |
| EGFR—Epidermal growth | tPr (total protein) |
| factor receptor | |
| Hepatocyte Growth Factor | VEGF Vascular endothelial |
| (HGF) | growth factor |
| ICAM-1—Intercellular | ANG-2—Angiopoietin-2 |
| adhesion molecule 1 | |
| TNF receptor superfamily | α-2-macroglobulin |
| member 5 | |
| Triiodothyronine | |

and naturally-occurring variants thereof.

In some embodiments, the kit further comprises at least one reagent specific for at least four biomarkers, in combination with at least one reagent specific for at least one other biomarker and/or at least one reagent specific for at least another biomarker, wherein the biomarkers are as herein described.

In some embodiments, the kit comprises at least one reagent specific for at least all biomarkers selected from the panel of markers.

In some embodiments, the kit further comprises at least one reagent specific for at least up to sixteen other biomarkers selected from the panel of markers for the other biomarkers.

In some embodiments, the kit further comprises at least one reagent specific for at least twenty five of the another biomarkers selected from the panel of markers for the another biomarkers.

In some embodiments, the kit comprises at least one reagent specific for:
Cortisol or a naturally-occurring variant thereof
IGF.BP.2—insulin-like growth factor binding protein 2 or a naturally-occurring variant thereof
IL.17—interleukin—17 or a naturally-occurring variant thereof
Pancreatic Polypeptide or a naturally-occurring variant thereof
ApoE ECU—apolipoprotein E or a naturally-occurring variant thereof
Calcium Corrected (Ca corr=Ca total+((40−alb)*0.02)) or a naturally-occurring variant thereof
ABeta 42 or a naturally-occurring variant thereof
Apolipoprotein E4 Allelle
VCAM−1—vascular cell adhesion molecule 1 or a naturally-occurring variant thereof

In some embodiments, the kit further comprises instructions for carrying out the method of diagnosing and/or aiding in the diagnosis of a neurological disorder in an individual and/or monitoring progression of a neurological disorder in an individual and/or stratifying an individual (i.e., sorting an individual with a probable diagnosis of a neurological disorder or diagnosed with a neurological disorder into different classes of the disorder), as herein described.

In some embodiments, the reagent specific for the biomarker is an antibody, or a fragment thereof, capable of detecting the biomarker. In some embodiments, the kit of the present invention includes a surface to which at least one reagent specific for said biomarker is attached. In some embodiments, the kit of the present invention includes a combination of a surface as herein described having attached thereto at least one reagent specific for a biomarker and a reference sample to which a test sample can be compared. The reference sample may be a biological sample from an individual (or a pooled sample from group of individuals) with a confirmed neurological disorder.

Kits comprising a single reagent specific for a biomarker will generally have the reagent enclosed in a container (e.g., a vial, ampoule, or other suitable storage container), although kits including the reagent bound to a substrate (e.g., an inner surface of an assay reaction vessel) are also contemplated. Likewise, kits including more than one reagent may also have the reagents in containers (separately or in a mixture) or may have the reagents bound to a substrate.

In some embodiments, the reagent(s) specific for a biomarker(s) will be labelled with a detectable marker (e.g., a fluorescent dye or a detectable enzyme), or be modified to facilitate detection (e.g., biotinylated to allow for detection with an avidin- or streptavidin-based detection system). In some embodiments, the reagent(s) specific for a biomarker(s) will not be directly labelled or modified.

Certain kits of the present invention will also include one or more agents for detection of bound biomarker-specific reagent (i.e., a reagent specific for a biomarker). As will be apparent to those skilled in the art, the identity of the detection agent(s) will depend on the type of biomarker-specific reagent(s) included in the kit and the intended detection system. Detection agents include antibodies (or fragments thereof) specific for the biomarker-specific reagent (e.g., secondary antibodies), primers for amplification of an biomarker-specific reagent that is nucleotide based (e.g., aptamer) or of a nucleotide ‘tag’ attached to the biomarker-specific reagent, avidin- or streptavidin-conjugates for detection of biotin-modified biomarker-specific reagent(s) and the like. Detection systems are well known in the art and need not be further described here.

A modified substrate or other system for capture of biomarkers may also be included in the kits of the present invention, particularly when the kit is designed for use in a sandwich-format assay. The capture system may be any capture system useful in a biomarker assay system, such as a multi-well plate coated with a biomarker-specific reagent(s), beads coated with a biomarker-specific reagent(s), and the like. Capture systems are well known in the art and need not be further described here.

In some embodiments, kits of the present invention include biomarker-specific reagent(s) in the form of an array. The array may include at least two different reagents specific for biomarkers (each reagent specific for a different biomarker) bound to a substrate in a predetermined pattern (e.g., a grid). Accordingly, the present invention also provides arrays comprising one or more reagents specific for at least four biomarkers are selected from a panel of markers consisting of:

| | | |
|---|---|---|
| Cortisol | SOD—superoxide | MPO—Myeloperoxidase |
| | dismutase | |
| IGF.BP.2—insulin-like | TIMP-1—tissue inhibitor of | Neut—neutrophils |
| growth factor binding | metalloproteinase 1 | |
| protein 2 | | |
| IL.17—interleukin - 17 | Adiponectin | PCV—packed cell volume |
| Pancreatic Polypeptide | BLC—chemokine (C—X—C | Rb85—Rubidium |
| | motif) ligand | |
| ApoE ECU—apolipoprotein | β2 Microglobin | RCC—red cell count |
| E | | |
| Calcium Corrected (Ca corr = | Cancer Antigen 19.9 | rFol—red cell folate |
| Ca total + ((40 − alb) * | | |
| 0.02)) | | |
| ABeta 42 | Eotaxin | Selenium |
| Apolipoprotein E4 Allelle | MIP-1-α—chemokine (C-C | TNF.RII—Tumor necrosis |
| | motif) ligand 3 | factor receptor superfamily |
| | | member 1B |
| VCAM-1—vascular cell | alb/tpr | tPr (total protein) |
| adhesion molecule 1 | | |
| Alb—albumin | CD40—CD40 molecule | VEGF Vascular endothelial |
| | | growth factor |
| B2M—beta-2-microglobulin | Chromium isotope | ANG-2—Angiopoietin-2 |
| | 52/Chromium isotope 53 | |
| CEA—carcinoembryonic | FT3 | α-2-macroglobulin |
| antigen | | |
| EGF.R—epidermal growth | HCY—homocysteine | EGFR—Epidermal growth |
| factor receptor | | factor receptor |
| Hb—haemoglobin | IL.10—interleukin 10 | Hepatocyte Growth Factor |
| | | (HGF) |
| Zinc | MCHC—mean cell | ICAM-1—Intercellular |
| | haemoglobin concentration | adhesion molecule 1 |
| Triiodothyronine | MMP.2—matrix | TNF receptor superfamily |
| | metallopeptidase 2 (72 kDa | member 5 |
| | type IV collagenase | |

and naturally-occurring variants thereof.

In another embodiment, at least two of the at least four biomarkers are selected from the group consisting of:
Cortisol
IGF.BP.2—insulin-like growth factor binding protein 2
IL.17—interleukin—17
Pancreatic Polypeptide
ApoE ECU—apolipoprotein E
Calcium Corrected (Ca corr=Ca total+((40−alb)*0.02))
ABeta 42
Apolipoprotein E4 Allelle
VCAM-1—vascular cell adhesion molecule 1
   and naturally-occurring variants thereof.

In some embodiments, the array further comprises one or more reagents specific for at least one other biomarker in combination with one or more reagents specific for the at least four biomarkers wherein the other biomarker is selected from a other panel of markers consisting of:

| | |
|---|---|
| Alb—albumin | SOD—superoxide |
| | dismutase |
| B2M—beta-2-microglobulin | TIMP-1—tissue inhibitor of |
| | metalloproteinase 1 |
| CEA—carcinoembryonic | Adiponectin |
| antigen | |
| EGF.R—epidermal growth | BLC—chemokine (C-X-C |
| factor receptor | motif) ligand |
| Hb—haemoglobin | β2 Microglobin |
| Zinc | Cancer Antigen 19.9 |
| IL.17—interleukin - 17 | Eotaxin |
| VCAM-1—vascular cell | MIP-1-α—chemokine (C-C |
| adhesion molecule 1 | motif) ligand 3 |

and naturally-occurring variants thereof.

In some embodiments, the array further comprises one or more reagents specific for at least another biomarker in combination with one or more reagents specific for the at least four biomarkers, wherein the at least another biomarker is selected from a panel of markers consisting of:

| | |
|---|---|
| alb/tpr | MPO—Myeloperoxidase |
| CD40—CD40 molecule | Neut—neutrophils |
| Chromium isotope | PCV—packed cell volume |
| 52/Chromium isotope 53 | |
| FT3 | Rb85—Rubidium |
| HCY—homocysteine | RCC—red cell count |
| IL.10—interleukin 10 | rFol—red cell folate |
| MCHC—mean cell | Selenium |
| haemoglobin concentration | |
| MMP.2—matrix | TNF.RII—Tumor necrosis |
| metallopeptidase 2 (72 kDa | factor receptor superfamily |
| type IV collagenase | member 1B |
| EGFR—Epidermal growth | tPr (total protein) |
| factor receptor | |
| Hepatocyte Growth Factor | VEGF Vascular endothelial |
| (HGF) | growth factor |
| ICAM-1—Intercellular | ANG-2—Angiopoietin-2 |
| adhesion molecule 1 | |
| TNF receptor superfamily | α-2-macroglobulin |
| member 5 | |
| Triiodothyronine | |

and naturally-occurring variants thereof.

In some embodiments, the present invention also provides arrays comprising one or more reagents specific for at least four biomarkers, alone or in combination with either or both of (i) one or more reagents specific for at least one other biomarker and (ii) one or more reagents specific for at least another biomarker wherein the biomarkers are as described herein.

In some embodiments, the array comprises one or more reagents specific for:
Cortisol or a naturally-occurring variant thereof
IGF.BP.2—insulin-like growth factor binding protein 2 or a naturally-occurring variant thereof
IL.17—interleukin—17 or a naturally-occurring variant thereof
Pancreatic Polypeptide or a naturally-occurring variant thereof
ApoE ECU—apolipoprotein E or a naturally-occurring variant thereof
Calcium Corrected (Ca corr=Ca total+((40−alb)*0.02)) or a naturally-occurring variant thereof
ABeta 42 or a naturally-occurring variant thereof
Apolipoprotein E4 Allelle
VCAM−1—vascular cell adhesion molecule 1 or a naturally-occurring variant thereof

In some embodiments, the array comprises one or more reagents specific for the biomarkers selected from any of the panel of markers described herein.

In some embodiments, the array further comprises at least one reagent specific for any number of markers up to sixteen other biomarkers selected from the panel of other markers.

In some embodiments, the array further comprises at least one reagent specific for any number of markers up to twenty five biomarkers selected from the panel of another markers.

Other examples of biomarkers and sets of biomarkers are described herein. The localization of the different biomarker-specific reagents (the “capture reagents”) allows measurement of levels of a number of different biomarkers in the same reaction. Kits including the reagents in array form are commonly in a sandwich format, so such kits may also comprise detection reagents. In some embodiments of the present invention, kits will include different detection reagents, each detection reagent specific to a different biomarker. The detection reagents in such embodiments are normally reagents specific for the same biomarkers as the reagents bound to the substrate (although the detection reagents typically bind to a different portion or site on the biomarker target than the substrate-bound reagents) and are generally affinity-type detection reagents. As with detection reagents for any other format assay, the detection reagents may be modified with a detectable moiety, modified to allow binding of a separate detectable moiety, or be unmodified. Array-type kits including detection reagents that are either unmodified or modified to allow binding of a separate detectable moiety may also contain additional detectable moieties (e.g., detectable moieties which bind to the detection reagent, such as labelled antibodies which bind unmodified detection reagents or streptavidin modified with a detectable moiety for detecting biotin-modified detection reagents).

In some embodiments of the present invention, the kits also comprise instructions for carrying out the method of diagnosing, aiding diagnosis and/or stratifying a neurological disorder in an individual and/or monitoring progression of a neurological disorder in an individual, as herein described.

The instructions relating to the use of the kit for carrying out the present invention generally describe how the contents of the kit are to be used to carry out the methods of the present invention. Instructions may include information as sample requirements (e.g., form, pre-assay processing and size), steps necessary to measure the biomarker(s) and how to interpretation of results.

Instructions supplied in the kits of the present invention are typically written instructions on a label or package insert (e.g., a paper sheet included in the kit), but machine-readable instructions (e.g., instructions carried on a magnetic or optical storage disk) are also envisaged. In some embodiments of the present invention, machine-readable instructions comprise software for a programmable digital computer for comparing the measured values obtained using the reagents included in the kit.

### Compositions

The present invention also provides a composition for use in the methods of the present invention (e.g., for diagnosing, aiding diagnosis and/or monitoring progression of a neurological disorder in an individual and/or stratifying an individual), the composition comprising at least one reagent specific for at least four biomarkers, wherein the at least four biomarkers are selected from a primary panel of markers consisting of:

| | | |
|---|---|---|
| Cortisol | SOD—superoxide | MPO—Myeloperoxidase |
| | dismutase | |
| IGF.BP.2—insulin-like | TIMP-1—tissue inhibitor of | Neut—neutrophils |
| growth factor binding | metalloproteinase 1 | |
| protein 2 | | |
| IL.17—interleukin - 17 | Adiponectin | PCV—packed cell volume |
| Pancreatic Polypeptide | BLC—chemokine (C—X—C | Rb85—Rubidium |
| | motif) ligand | |
| ApoE ECU—apolipoprotein | β2 Microglobin | RCC—red cell count |
| E | | |
| Calcium Corrected (Ca corr = | Cancer Antigen 19.9 | rFol—red cell folate |
| Ca total + ((40 − alb) * | | |
| 0.02)) | | |
| ABeta 42 | Eotaxin | Selenium |
| Apolipoprotein E4 Allelle | MIP-1-α—chemokine (C-C | TNF.RII—Tumor necrosis |
| | motif) ligand 3 | factor receptor superfamily |
| | | member 1B |
| VCAM-1—vascular cell | alb/tpr | tPr (total protein) |
| adhesion molecule 1 | | |
| Alb—albumin | CD40—CD40 molecule | VEGF Vascular endothelial |
| | | growth factor |
| B2M—beta-2-microglobulin | Chromium isotope | ANG-2—Angiopoietin-2 |
| | 52/Chromium isotope 53 | |
| CEA—carcinoembryonic | FT3 | α-2-macroglobulin |
| antigen | | |
| EGF.R—epidermal growth | HCY—homocysteine | EGFR—Epidermal growth |
| factor receptor | | factor receptor |
| Hb—haemoglobin | IL.10—interleukin 10 | Hepatocyte Growth Factor |
| | | (HGF) |
| Zinc | MCHC—mean cell | ICAM-1—Intercellular |
| | haemoglobin concentration | adhesion molecule 1 |
| Triiodothyronine | MMP.2—matrix | TNF receptor superfamily |
| | metallopeptidase 2 (72 kDa | member 5 |
| | type IV collagenase | |

and naturally-occurring variants thereof.

In another embodiment, at least two of the at least four biomarkers are selected from the group consisting of:
Cortisol
IGF.BP.2—insulin-like growth factor binding protein 2
IL.17—interleukin—17
Pancreatic Polypeptide
ApoE ECU—apolipoprotein E —Calcium Corrected (Ca corr=Ca total+((40−alb)*0.02))
ABeta 42
Apolipoprotein E4 Allelle
VCAM−1—vascular cell adhesion molecule 1
   and naturally-occurring variants thereof.

In some embodiments, the composition further comprises at least one reagent specific for at least one other biomarker in combination with at least one reagent specific for the at least four biomarkers, wherein the at least one one other biomarker is selected from a other panel of markers consisting of:

| | |
|---|---|
| Alb—albumin | SOD—superoxide |
| | dismutase |
| B2M—beta-2-microglobulin | TIMP-1—tissue inhibitor of |
| | metalloproteinase 1 |
| CEA—carcinoembryonic | Adiponectin |
| antigen | |
| EGF.R—epidermal growth | BLC—chemokine (C-X-C |
| factor receptor | motif) ligand |
| Hb—haemoglobin | β2 Microglobin |
| Zinc | Cancer Antigen 19.9 |
| IL.17—interleukin - 17 | Eotaxin |
| VCAM-1—vascular cell | MIP-1-α—chemokine (C-C |
| adhesion molecule 1 | motif) ligand 3 |

and naturally-occurring variants thereof.

In some embodiments, the composition further comprises at least one reagent specific for at least another biomarker in combination with at least one reagent specific for at least four biomarkers, wherein the at least another biomarker is selected from a tertiary panel of markers consisting of:

| | |
|---|---|
| alb/tpr | MPO—Myeloperoxidase |
| CD40—CD40 molecule | Neut—neutrophils |
| Chromium isotope | PCV—packed cell volume |
| 52/Chromium isotope 53 | |
| FT3 | Rb85—Rubidium |
| HCY—homocysteine | RCC—red cell count |
| IL.10—interleukin 10 | rFol—red cell folate |
| MCHC—mean cell | Selenium |
| haemoglobin concentration | |
| MMP.2—matrix | TNF.RII—Tumor necrosis |
| metallopeptidase 2 (72 kDa | factor receptor superfamily |
| type IV collagenase | member 1B |
| EGFR—Epidermal growth | tPr (total protein) |
| factor receptor | |
| Hepatocyte Growth Factor | VEGF Vascular endothelial |
| (HGF) | growth factor |
| ICAM-1—Intercellular | ANG-2—Angiopoietin-2 |
| adhesion molecule 1 | |
| TNF receptor superfamily | α-2-macroglobulin |
| member 5 | |
| Triiodothyronine | |

and naturally-occurring variants thereof.

In some other embodiments, the present invention provides a composition for use in diagnosing, aiding diagnosis and/or monitoring progression of a neurological disorder in an individual and/or stratifying (i.e., sorting an individual with a probable diagnosis of a neurological disorder or diagnosed with a neurological disorder into different classes of the disorder) an individual, the kit comprising at least one reagent specific for at least four biomarkers, and at least one reagent specific for at least one of the other and/or tertiary biomarkers wherein the biomarkers are as described herein.

In some embodiments, the composition further comprises at least one reagent specific for at least up to all of the biomarkers listed.

In some embodiments, the composition further comprises at least one reagent specific for at least up to sixteen of the other biomarkers selected from the other panel of markers.

In some embodiments, the composition further comprises at least one reagent specific for at least up to twenty five of the another biomarkers selected from the panel of the another markers.

In some embodiments, the composition comprises at least one reagent specific for:
Cortisol or a naturally-occurring variant thereof
IGF.BP.2—insulin-like growth factor binding protein 2 or a naturally-occurring variant thereof
IL.17—interleukin—17 or a naturally-occurring variant thereof
Pancreatic Polypeptide or a naturally-occurring variant thereof
ApoE ECU—apolipoprotein E or a naturally-occurring variant thereof
Calcium Corrected (Ca corr=Ca total+((40−alb)*0.02)) or a naturally-occurring variant thereof
ABeta 42 or a naturally-occurring variant thereof
Apolipoprotein E4 Allelle
VCAM−1—vascular cell adhesion molecule 1 or a naturally-occurring variant thereof

In another aspect, the present invention provides a composition comprising one or more of the biomarkers as herein described (e.g., for use as reference samples and/or as appropriate controls).

The present invention also provides a system of diagnosing or aiding diagnosis of a neurological disorder and/or monitoring a neurological disorder, the system comprising a computational means for comparing a measured level of at least four biomarkers in a biological sample from an individual to a reference level for the at least four biomarkers, wherein the at least four biomarkers are selected from a primary panel of markers consisting of:

| | | |
|---|---|---|
| Cortisol | SOD—superoxide | MPO—Myeloperoxidase |
| | dismutase | |
| IGF.BP.2—insulin-like | TIMP-1—tissue inhibitor of | Neut—neutrophils |
| growth factor binding | metalloproteinase 1 | |
| protein 2 | | |
| IL.17—interleukin - 17 | Adiponectin | PCV—packed cell volume |
| Pancreatic Polypeptide | BLC—chemokine (C—X—C | Rb85—Rubidium |
| | motif) ligand | |
| ApoE ECU—apolipoprotein | β2 Microglobin | RCC—red cell count |
| E | | |
| Calcium Corrected (Ca corr = | Cancer Antigen 19.9 | rFol—red cell folate |
| Ca total + ((40 − alb) * | | |
| 0.02)) | | |
| ABeta 42 | Eotaxin | Selenium |
| Apolipoprotein E4 Allelle | MIP-1-α—chemokine (C-C | TNF.RII—Tumor necrosis |
| | motif) ligand 3 | factor receptor superfamily |
| | | member 1B |
| VCAM-1—vascular cell | alb/tpr | tPr (total protein) |
| adhesion molecule 1 | | |
| Alb—albumin | CD40—CD40 molecule | VEGF Vascular endothelial |
| | | growth factor |
| B2M—beta-2-microglobulin | Chromium isotope | ANG-2—Angiopoietin-2 |
| | 52/Chromium isotope 53 | |
| CEA—carcinoembryonic | FT3 | α-2-macroglobulin |
| antigen | | |
| EGF.R—epidermal growth | HCY—homocysteine | EGFR—Epidermal growth |
| factor receptor | | factor receptor |
| Hb—haemoglobin | IL.10—interleukin 10 | Hepatocyte Growth Factor |
| | | (HGF) |
| Zinc | MCHC—mean cell | ICAM-1—Intercellular |
| | haemoglobin concentration | adhesion molecule 1 |
| Triiodothyronine | MMP.2—matrix | TNF receptor superfamily |
| | metallopeptidase 2 (72 kDa | member 5 |
| | type IV collagenase | |

and naturally-occurring variants thereof.

In another embodiment, at least two of the at least four biomarkers are selected from the group consisting of:
Cortisol
IGF.BP.2—insulin-like growth factor binding protein 2
IL.17—interleukin—17
Pancreatic Polypeptide
ApoE ECU—apolipoprotein E
Calcium Corrected (Ca corr=Ca total+((40−alb)*0.02))
ABeta 42
Apolipoprotein E4 Allelle
VCAM-1—vascular cell adhesion molecule 1
   and naturally-occurring variants thereof.

The present invention also provides a method of treating an individual for a neurological disorder, the method comprising obtaining a biological sample from an individual; comparing a measured level of at least four biomarkers in the biological sample to a reference level for the at least four biomarkers, wherein the at least four biomarkers are selected from a primary panel of markers consisting of:

| | | |
|---|---|---|
| Cortisol | SOD—superoxide | MPO—Myeloperoxidase |
| | dismutase | |
| IGF.BP.2—insulin-like | TIMP-1—tissue inhibitor of | Neut—neutrophils |
| growth factor binding | metalloproteinase 1 | |
| protein 2 | | |
| IL.17—interleukin-17 | Adiponectin | PCV—packed cell volume |
| Pancreatic Polypeptide | BLC—chemokine (C—X—C | Rb85—Rubidium |
| | motif) ligand | |
| ApoE ECU—apolipoprotein | β2 Microglobin | RCC—red cell count |
| E | | |
| Calcium Corrected (Ca corr = | Cancer Antigen 19.9 | rFol—red cell folate |
| Ca total + ((40 − alb) * | | |
| 0.02)) | | |
| ABeta 42 | Eotaxin | Selenium |
| Apolipoprotein E4 Allelle | MIP-1-α—chemokine (C-C | TNF.RII—Tumor necrosis |
| | motif) ligand 3 | factor receptor superfamily |
| | | member 1B |
| VCAM-1—vascular cell | alb/tpr | tPr (total protein) |
| adhesion molecule 1 | | |
| Alb—albumin | CD40—CD40 molecule | VEGF Vascular endothelial |
| | | growth factor |
| B2M—beta-2-microglobulin | Chromium isotope | ANG-2—Angiopoietin-2 |
| | 52/Chromium isotope 53 | |
| CEA—carcinoembryonic | FT3 | α-2-macroglobulin |
| antigen | | |
| EGF.R—epidermal growth | HCY—homocysteine | EGFR—Epidermal growth |
| factor receptor | | factor receptor |
| Hb—haemoglobin | IL.10—interleukin 10 | Hepatocyte Growth Factor |
| | | (HGF) |
| Zinc | MCHC—mean cell | ICAM-1—Intercellular |
| | haemoglobin concentration | adhesion molecule 1 |
| Triiodothyronine | MMP.2—matrix | TNF receptor superfamily |
| | metallopeptidase 2 (72 kDa | member 5 |
| | type IV collagenase | |

and naturally-occurring variants thereof;
and, where there is a difference in the measured level of the at least four biomarkers compared to the reference level of the at least four biomarkers, indicative of a neurological disorder or severity of a neurological disorder, administering to the individual a therapeutically effective amount of an agent capable of alleviating a symptom of the neurological disorder. Exemplary agents include, but are not limited to, cholinesterase inhibitors (e.g., galantamine, rivastigmine, donepezil) and N-methyl D-aspartate (NMDA) antagonists (e.g., memantine).

In another embodiment, at least two of the at least four biomarkers are selected from the group consisting of:
Cortisol
IGF.BP.2—insulin-like growth factor binding protein 2
IL.17—interleukin—17
Pancreatic Polypeptide
ApoE ECU—apolipoprotein E
Calcium Corrected (Ca corr=Ca total+((40−alb)*0.02))
ABeta 42
Apolipoprotein E4 Allelle
VCAM−1—vascular cell adhesion molecule 1
   and naturally-occurring variants thereof.

The following Examples are provided to illustrate the invention, but are not intended to limit the scope of the present invention in any way.

### EXAMPLES

### Statistical Analysis of Biomarker Data from the Australian Imaging Biomarkers and Lifestyle (AIBL) Study

### A. Introduction

As part of the AIBL study, measurements of 151 biomarkers were taken from 1113 volunteer participants who had been classified as:
Diagnosed with Alzheimer's Disease (AD) (211 participants)
Diagnosed with Mild Cognitive impairment (MCI) (134 participants)
Health Controls (HC) (768 participants)

The data were statistically analysed to identify a small panel of biomarkers that could distiguish AD from HC. The MCI group was not included in the study.

### B. Data Cleaning and Outlier Checking

The dataset was cleaned before analysis by:

(a) replacing biomarker values recorded as below detection limits with a small positive value;
(b) removing clearly erroneous values. Values identified by inspection of descriptive statistics and diagnostic plots of the data as clearly incompatible with the main bulk of the data were removed and replaced by the median value for the biomarker; and
(c) imputing values for missing data using multivariate normal imputation (Schafer, J. L. (1997) Analysis of Incomplete Multivariate Data. Chapman & Hall, London) with five-fold replication, so that 5 similar datasets were generated each with different values imputed for the missing data.

Separate analyses were conducted for each of the five sets so that the robustness to missing data could be assessed.

Of the 151 biomarkers, 17 were found to have missing values for 60% or more of participants. These were excluded from further analysis, leaving 134 biomarkers in the study.

Inspection of descriptive statistics and diagnostic plots indicated substantial skewness in the distributions of the biomarker values. This was reduced by log transforming all the biomarker values.

### C. Statistical Modelling

Several different analysis approaches were used to identify formulae that distinguish between AD and HC participants on the basis of a small subset of their biomarker values. The use of multiple methods increases the robustness of the conclusions about the usefulness of the final set of biomarkers, since each method brings a different bias. Biomarkers selected by multiple methods are more likely to provide valid predictions.

A training set/test set approach was used so that the data used to fit the models was separate from that used to test their performance as predictors. The groups of AD cases and HC participants were each divided into a training set consisting of 70% of the group and a test set consisting of the remaining 30% of the group. The models were fitted to the training set and their performance evaluated on the test set. The fitting and testing was repeated five times, once for each of the five imputed datasets.

Four methods were used to identify a small subset of biomarkers giving good discrimination between AD and HC. These were:

1. Random Forests (RF) (Breiman, Leo (2001). “Random Forests”. Machine Learning 45 (1): 5-32; incorporated herein by reference in its entirety);
2. Linear Models for Micro Array data (LIMMA) (G. K. Smyth. Linear models and empirical bayes methods for assessing differential expression in microarray experiments. Statistical Applications in Genetics and Molecular Biology, 3, 2004; incorporated herein by reference in its entirety);
3. Classification Trees (CT) (Breiman, Leo; Friedman, J. H., Olshen, R. A., & Stone, C. J. (1984), Classification and regression trees, Monterey, Calif.: Wadsworth & Brooks/Cole Advanced Books & Software; incorporated herein by reference in its entirety); and
4. Boosted Trees (BT) ([2] J.H. Friedman (2001). “Greedy Function Approximation: A Gradient Boosting Machine,” Annals of Statistics 29(5):1189-1232; incorporated herein by reference in its entirety).

The use of multiple methods makes the resulting selection more robust to the detail of the models fitted.

### 1. Random Forests

RF (classification) is a variable selection method that uses classification trees to infer class membership to each case. RF grows a number of classification trees (a forest), and counts the number of votes from trees (each tree provides a vote for a specific class) to predict class membership. RF measures the impact of each biomarker by a ‘variable importance’, which is a relative measure on how well each variable is able to predict the class membership. The randomForest package for the R statistical package (A. Liaw and M. Wiener (2002). Classification and Regression by randomForest. R News 2(3), 18-22) was used to fit the model.

2. Linear Models for Micro Array data analysis (LIMMA)

The LIMMA method has been widely used in the analysis of micro array data. Its general purpose to identify gene expression difference between two classes where there are many more variables than observations). The method starts with fitting a standard linear model to the data, and then uses an Empirical Bayes approach to borrow information across variables (reduction of sample error), and uses a moderated t-statistic with an augmented degrees of freedom. The LIMMA method outputs a False Discovery Rate (FDR) adjusted p-value (the ‘q-value’) for each biomarker which indicates its value as a predictor. The LIMMA program for the R statistical package was used for this study (Smyth, G. K. (2005). Limma: linear models for microarray data. In: Bioinformatics and Computational Biology Solutions using R and Bioconductor, R. Gentleman, V. Carey, S. Dudoit, R. Irizarry, W. Huber (eds.), Springer, N.Y., pages 397-420.)

### 3. Classification Trees

The CT method is an alternative approach to a non-linear regression where there are many complex interactions between multiple variables, whether they are continuous or categorical in nature. The method creates multiple partitions or subdivisions of data (recursive partitioning) so that the interaction between multiple variables becomes simpler. Recursive partitioning is analogous to creating multiple classification trees, where the interior branches are questions, and the outer leaves are the answers to the questions. The final tree uses only a subset of the variables. The rpart command within the R statistical package was used for this study (R Development Core Team (2009). R: A language and environment for statistical computing. R Foundation for Statistical Computing, Vienna, Austria. ISBN 3-900051-07-0, URL http://www.R-project.org.)

### 4. Generalized Boosted Regression Modelling (Boosted Trees)

BT is a variable selection and class prediction method that builds an initial binary classification tree (a root node and two child nodes), and then fits another tree based upon the partition residuals from the prior tree. This computation is iterated many times, and acts as a weighted remodelling process prior to votes for class prediction are totalled from all trees. BT outputs a relative influence measure that, similar to the variable importance, provides a relative measure on how well each variable is able to predict class membership. The gmb command within the R statistical package was used for this study (R Development Core Team (2009). R: A language and environment for statistical computing. R Foundation for Statistical Computing, Vienna, Austria. ISBN 3-900051-07-0, URL http://www.R-project.org).

Each method gives an indicator of the value of each biomarker for discrimination: the variable importance in RF, the q-value in LIMMA, inclusion/exclusion in CT and relative influence in BT. These indicators were averaged over the five datasets created by imputation. The top 30 biomarkers identified by these averaged indicators for each of RF, LIMMA and BT method are given in Table 1, together with the 15 biomarkers included in the CT model, while Table 2 gives the 25 biomarkers that were selected by two or more of the methods.

TABLE 1
Top 30 Biomarkers identified by BT, RF and LIMMA and Biomarkers
selected by CT

| | | | |
|---|---|---|---|
| Boosted Trees Top 30 | Random Forest Top 30 | | |
| Relative Influence | Relative Importance | LIMMA Adjusted q value | Classification Tree |

| | | | |
|---|---|---|---|
| [symbol] | [symbol] | [symbol] | [symbol] |
| [symbol] | [symbol] | TNF.RII | IL.17 |
| MMP.9 | [symbol] | VEGF | [symbol] |
| CTGF | TIMP.1 | Beta.2.Microglobulin | [symbol] |
| B.Lymphocyte.Chemoattractant..BLC. | [symbol] | TIMP.1 | Cancer.Antigen.19.9 |
| ICAM.1 | [symbol] | EGF.R | Serum.Amyloid.P |
| SOD | IL.17 | [symbol] | [symbol] |
| EN.RAGE | VEGF | CD40 | [symbol] |
| Carcinoembryonic.Antigen | EGF.R | IL.17 | TIMP.1 |
| [symbol] | Carcinoembryonic.Antigen | [symbol] | von.Willebrand.Factor |
| [symbol] | Cancer.Antigen.19.9 | Fatty.Acid.Binding.Protein | PAPP.A |
| Apolipoprotein.E | E2 | CgA | Ciliary.Neurotrophic.- |
| | | | Factor.CNTF. |
| Thyroid.Stimulating.Hormone | TNF.RII | Myoglobin | Eotaxin |
| Eotaxin | HCC.4 | [symbol] | Glucagon |
| Beta.2.Microglobulin | Eotaxin | B.Lymphocyte.Chemoattractant..BLC. | IgE |
| IL.5 | Beta.2.Microglobulin | [symbol] | |
| LH..Luteinizing.Hormone. | B.Lymphocyte.Chemoattractant..BLC. | Sortilon | |
| IgA | SHBG | IL.10 | |
| Myeloperoxidase | Angiopoietin.2..ANG.2. | Carcinoembryonic.Antigen | |
| [symbol] | CD40 | ICAM.1 | |
| Alpha.2.Macroglobulin | MMP.2 | Angiopoietin.2..ANG.2. | |
| Adiponectin | Apolipoprotein.D | Hepatocyte.Growth.Factor..HGF. | |
| Complement.Factor.H | Haptoglobin | MIP.1alpha | |
| IL.16 | Testosterone | IL.8 | |
| MMP.2 | Apolipoprotein.A1 | PYY | |
| MIP.1alpha | Hepatocyte.Growth.Factor..HGF. | E2 | |
| C.Reactive.Protein | Adiponectin | Tenascin.C | |
| MDC | Apolipoprtein.B | TNF.alpha | |
| NrCAM | Myeloperoxidase | FAS | |
| Cancer.Antigen.19.9 | MIP.1alpha | Adiponectin | |

Biomarkers in bold italic were selected by all four methods, those in bold by three of the methods, those in italic by two of the methods and those in normal text by only one method.

TABLE 2
Top 25 Biomarkers including age and ApoE4 genotype

age
Adiponectin
Angiopoietin.2..ANG.2.
B.Lymphocyte.Chemoattractant..BLC.
Beta.2.Microglobulin
Cancer.Antigen.19.9
Carcinoembryonic.Antigen
CD40
Cortisol
E2
E4
EGF.R
Eotaxin
Hepatocyte.Growth.Factor..HGF.
ICAM.1
IGF.BP.2
IL.17
MIP.1alpha
MMP.2
Myeloperoxidase
Pancreatic.polypeptide
TIMP.1
TNF.RII
VCAM.1
VEGF

Table 3 gives the 5 biomarkers selected by all four of the methods, together with two that were close to the top of the list for all methods except BT. Age is also included in the list since it clearly affects the likelihood of an individual being diagnosed with AD.

TABLE 3
Top 8 Biomarkers including age and ApoE4 genotype

age
Cortisol
IGF.BP.2
E4
IL.17
Pancreatic.polypeptide
TIMP.1
VCAM.1

### D. Predictive Models and Model Validation

Having identified the 8 biomarkers of greatest value for prediction, three different methods were used to determine predictive functions of these biomarkers that can be used to classify new individuals as AD or HC. The predictive function can be calculated on data from a new individual and the new individual can be assessed as AD or HC according to whether the predictive function value is above or below a predefined cutoff value. The cutoff value can be chosen to achieve a balance between Sensitivity, the probability that an AD case is assessed as AD, and Specificity, the probability that a HC is assessed as HC. The RF and BT methods as described above were applied, together with Linear Discriminant Analysis (LDA). The LDA method has the advantage that the predictive function is an easily calculated function of the biomarker values, whereas RF and BT require special software for their evaluation. Thus if the performance of LDA is comparable with RF and BT it would have advantages in practical application of the predictor.

It was found that there was missing data on the 8 biomarkers for only 15 of the 979 participants. Therefore in the predictive modeling and validation, data for these 15 participants was excluded to avoid any impact of imputation on the conclusions.

The three models were fitted to a 70% training set as used above and the performance of the models was then tested using the 30% test sets that had been excluded from the model fitting procedures. This procedure was repeated 5 times with different randomly chosen training and test sets. Thus the conclusions are not biased by the fitting process.

The performance was measured using the Receiver Operating Characteristic (ROC) curve, a graph of the sensitivity versus the specificity of a test based on a function of the biomarkers for all possible cutoff values. (Pepe MS. (2003) The Statistical Evaluation of Medical Tests for Classification and Prediction. Oxford University Press, pp 67-68).

### E. Results

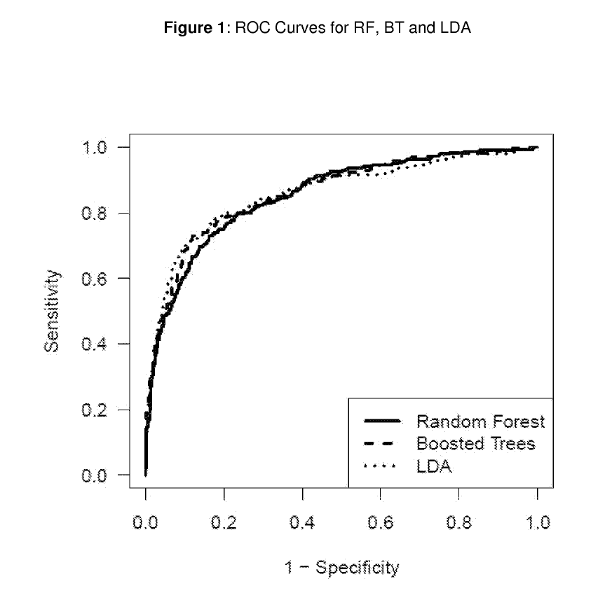

The ROC Curves for RF, BT and LDA are plotted in FIG. 1. It can be seen from FIG. 1 that the LDA curve shows comparable performance to the RF and BT curves. In particular the LDA curve is above the RF and BT curves in much of the region around a specificity of 0.8 to 0.9 (1-specificity=0.1 to 0.2). This indicates that the simpler LDA model will give good performance for tests with cutoff values in this region, which is often that of most interest.

Table 4 gives the sensitivity and specificity for the three methods for cutoff points chosen to give Sensitivity=Specificity. The Area Under the Curve (AUC) statistic commonly used to compare ROC curves is also included. All three methods give good performance and again the LDA method is seems slightly better than the others.

TABLE 4
Sensitivity, Specificity and AUC for RF, BT and LDA

| | Sensitivity/ | |
|---|---|---|
| | Specificy | AUC |
| Random Forest | 0.78 | 0.86 |
| Boosted Trees | 0.78 | 0.87 |
| LDA | 0.79 | 0.86 |

The coefficients in the fitted LDA model are given in Table 5. The values are positive for all biomarkers except IL-17, indicating that AD risk decreases with increasing IL17 concentration, but increases with increasing age, increasing concentrations of the biomarkers other than IL-17 and is higher for carriers of the E4 allele of APOE.

TABLE 5
Coefficients in fitted LDA model

| Biomarker | Coefficient |
|---|---|
| age | 0.055 |
| Cortisol | 1.255 |
| E4 | 1.139 |
| IGF.BP.2 | 0.393 |
| IL-17 | −1.197 |
| Pancreatic.polypeptide | 0.448 |
| TIMP.1 | 0.173 |
| VCAM.1 | 0.647 |

### F. Conclusions

The selection of a set of 6 biomarkers from a set of 151 biomarkers (together with ApoE status and Age) provides a simple predictor of AD status with good sensitivity and specificity. The use of a weighted average of the biomarkers developed using LDA is suitable to implement this predictor.

### G. Clinical Diagnosis of Alzheimer's Disease

A patient would typically arrive at a memory clinic having been referred with a history of cognitive decline. Current investigative processes include history taking, examination and collateral informant history. Subsequent investigations may include neuropsychology, imaging and blood tests as required from history and examination findings.

The present invention provides the clinician with an improved means of diagnosing or aiding in the diagnosis of AD or other neurological disorder. The methods of the present invention can be performed alone or in combination with existing means of diagnosis. For example, the clinician would collect a biological fluid sample (e.g., blood) from the patient and send the sample off to a diagnostic laboratory to perform the method of the present invention. The results will provide serum levels of the biochemical markers in the panel and a probability of cognitive decline, development of AD and/or other neurological disorder(s).

Clinicians can use this information as a guide to assess the degree of cognitive decline, development of AD and/or other neurological disorder(s), thus contributing to their management of their patients' health.

Knowing a degree of cognitive decline and development of AD and/or other neurological disorder(s) may assist in:
Consideration of therapy;
Inclusion in trials for new therapies delaying onset of AD and/or other neurological disorder;
Consideration on whether or not to move on to more invasive diagnostic tests (i.e. lumbar puncture, imaging using radiation);
Consideration for rigorous physical activity and antioxidant program interventions with some evidence to delay cognitive decline;
Planning for later (e.g., asset management, planning for medical and legal power of attorney, lifestyle adjustments, etc.)

While there is no clear therapy or preventive program at present, the capacity to identify individuals with a neurological disorder by the present invention may lead to the development of new intervention and preventative measures.

### H. Material and Methods for Analysing a Blood Sample

A blood sample will be taken and forwarded to a clinical pathology laboratory for testing. Stored blood samples were sourced from 3 different tube types: lithium-heparin tubes, EDTA tubes with added prostaglandin E1 (Sapphire Biosciences, 33.3 ng/ml) and serum tubes.

Blood samples were processed for plasma for use in a commercially available biomarker detection assay (e.g., ELISA). Blood samples were centrifuged at 1800 g for 15 minutes at room temperature and the plasma was transferred to a polypropylene tube and stored in liquid nitrogen until analysis. A 0.5 ml aliquot that had not been subject to any freeze-thaw cycle was shipped to Rules Based Medicine (RBM, Austin, Tex.) for analysis. No patient samples were older than 18 months at the time of analysis.

### I. Luminex xMAP Panel

Plasma samples were analyzed using a commercially available multiplexed luminex human discovery xMAP panel from Rules Based Medicine (RBM, Austin, Tex.). All assays were validated according to CLIA standards. In brief, the luminex technology multiplexes immunoassays on the surface of polystyrene microsphere beads. The microsphere beads are loaded with a ratio of two spectrally distinct fluorochromes yielding up to 100 uniquely colour-coded beads. The beads were coated with capture antibodies specific for the assay and run in either a standard sandwich or competitive immunoassay format. Capture-antibody microspheres were incubated with blocking solution and diluted plasma sample or calibration controls for one hour. Beads were rinsed and biotinylated detection reagent was added. Streptavidin-phycoerthyrin was then added to each well and incubated for 60 minutes. Following additional wash steps, the beads were resuspended in reading solution and read on the luminex instrument.

Some of the assays defined a lower limit of quantitation. For the purposes of this experiment, the lower limit of detection (LD) was utilized. The LLD was determined by analyzing 20 diluted blank samples (made of plasma matrix), calculating the mean background and adding 3 standard deviations to the mean. The AIBL dataset was analyzed with a 151 biomarker multiplex panel.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention before the priority date of each claim of this application.

Finally it is to be understood that various other modifications and/or alterations may be made without departing from the spirit of the present invention as outlined herein.

Future patent applications may be filed on the basis of or claiming priority from the present application. It is to be understood that the following provisional claims are provided by way of example only, and are not intended to limit the scope of what may be claimed in any such future application. Features may be added to or omitted from the provisional claims at a later date so as to further define or re-define the invention or inventions.

1-48. (canceled)

49. A kit for use in diagnosing, aiding diagnosis and/or monitoring progression of a neurological disorder in an individual and/or stratifying an individual, the kit comprising at least one reagent specific for at least six biomarkers, wherein the at least six biomarkers are selected from a panel of markers consisting of:
Cortisol
IGF.BP.2—insulin-like growth factor binding protein 2
IL.17—interleukin—17
Pancreatic Polypeptide
ApoE ECU—apolipoprotein E
ABeta 42
VCAM−1—vascular cell adhesion molecule 1
BLC—chemokine (C-X-C motif) ligand
and naturally-occurring variants thereof.

50. The kit of claim 49, further comprising at least one reagent specific for at least one other biomarker, wherein the at least one other biomarker is selected from a panel of markers consisting of:

| | |
|---|---|
| Alb—albumin | SOD—superoxide dismutase |
| B2M—beta-2-microglobulin | TIMP-1—tissue inhibitor of |
| | metalloproteinase 1 |
| CEA—carcinoembryonic | Adiponectin |
| antigen | |
| EGF.R—epidermal growth | BLC—chemokine (C—X—C |
| factor receptor | motif) ligand |
| Hb—haemoglobin | β2 Microglobin |
| Zinc | Cancer Antigen 19.9 |
| IL.17—interleukin - 17 | Eotaxin |
| VCAM -1—vascular cell | MIP-1-α—chemokine (C-C |
| adhesion molecule 1 | motif) ligand 3 |
| Cortisol | IGF.BP.2—insulin-like |
| | growth factor binding protein 2 |
| Pancreatic Polypeptide | ApoE ECU—apolipoprotein E |
| ABeta 42 | |

and naturally-occurring variants thereof.

51. The kit of claim 50, further comprising at least one reagent specific for at least another biomarker, wherein the at least another biomarker is selected from a panel of markers consisting of:

| | |
|---|---|
| alb/tpr | MPO—Myeloperoxidase |
| CD40—CD40 molecule | Neut—neutrophils |
| Chromium isotope | PCV—packed cell volume |
| 52/Chromium isotope 53 | |
| FT3 | Rb85—Rubidium |
| HCY—homocysteine | RCC—red cell count |
| IL.10—interleukin 10 | rFol—red cell folate |
| MCHC—mean cell | Selenium |
| haemoglobin concentration | |
| MMP.2—matrix | TNF.RII—Tumor necrosis |
| metallopeptidase 2 (72 kDa | factor receptor superfamily |
| type IV collagenase | member 1B |
| EGFR—Epidermal growth | tPr (total protein) |
| factor receptor | |
| Hepatocyte Growth Factor | VEGF Vascular endothelial |
| (HGF) | growth factor |
| ICAM-1—Intercellular | ANG-2—Angiopoietin-2 |
| adhesion molecule 1 | |
| TNF receptor superfamily | α-2-macroglobulin |
| member 5 | |
| Triiodothyronine | Calcium Corrected (Ca corr = |
| | Ca total + ((40 − alb) * |
| | 0.02)) |
| Apolipoprotein E4 Allelle | |

and naturally-occurring variants thereof.

52. The kit of claim 49 wherein the use in diagnosing, aiding diagnosis and/or monitoring progression of a neurological disorder in an individual and/or stratifying an individual comprises comparing a measured level of the at least six biomarkers and naturally-occurring variants thereof in a biological sample from an individual to a reference level for the at least six biomarkers and naturally-occurring variants thereof.

53. The kit of claim 52, wherein comparing the measured level of the at least six biomarkers in the biological sample from the individual comprises comparing the measured level of:
i) Abeta42, ApoE, VCAM1, BLC—chemokine (C-X-C motif) ligand, Pancreatic polypeptide, IL-17, or their naturally-occurring variants thereof;
ii) Abeta42, ApoE, Cortisol, BLC—chemokine (C-X-C motif) ligand, Pancreatic polypeptide, IL-17, or their naturally-occurring variants thereof;
iii) Abeta42, ApoE, Cortisol, VCAM1, Pancreatic polypeptide, IL-17, or their naturally-occurring variants thereof, or their naturally-occurring variants thereof;
iv) Abeta42, ApoE, Cortisol, VCAM1, BLC—chemokine (C-X-C motif) ligand, IL-17, or their naturally-occurring variants thereof; and
v) Abeta42, ApoE, Cortisol, VCAM1, BLC—chemokine (C-X-C motif) ligand, Pancreatic polypeptide, or their naturally-occurring variants thereof

54. The kit of claim 49, wherein the neurological disorder is Alzheimer's disease.

55. The kit of claim 52 wherein the comparing of a measured level of at least six biomarkers in a biological sample from an individual to a reference level for the at least six biomarkers is carried out by one or more of the statistical methods selected from the group consisting of Random Forest, Support Vector Machine, Linear Models for MicroArray data (LIMMA) and/or Significance Analyses of Microarray Data (SAM), Best First, Greedy Stepwise, Naive Bayes, Linear Forward Selection, Scatter Search, Linear Discriminant Analysis (LDA), Stepwise Logistic Regression, Receiver Operating Characteristic and Classification Trees (CT).

56. The kit of claim 52 wherein comparing the measured levels for each biomarker is carried out using Boosted Trees (BT) and wherein the comparing provides sensitivity of at least 85% and specificity of at least 85% in diagnosing or aiding diagnosis of a neurological disorder in an individual.

57. A method of diagnosing, aiding diagnosis, stratifying an individual into one or more classes, or monitoring progression of a neurological disorder, the method comprising comparing a measured level of at least six biomarkers in a biological sample from an individual to a reference level for the at least six biomarkers, wherein the at least six biomarkers are selected from a panel of markers consisting of:
Cortisol
IGF.BP.2—insulin-like growth factor binding protein 2
IL.17—interleukin—17
Pancreatic Polypeptide
ApoE ECU—apolipoprotein E
ABeta 42
VCAM−1—vascular cell adhesion molecule 1
BLC—chemokine (C-X-C motif) ligand
and naturally-occurring variants thereof.

58. The method of claim 57, further comprising comparing a measured level of at least one other biomarker in a biological sample from the individual to a reference level for the at least one other biomarker, wherein the at least one other biomarker is selected from a panel of markers consisting of:

| | |
|---|---|
| Alb—albumin | SOD—superoxide dismutase |
| B2M—beta-2-microglobulin | TIMP-1—tissue inhibitor of |
| | metalloproteinase 1 |
| CEA—carcinoembryonic | Adiponectin |
| antigen | |
| EGF.R—epidermal growth | BLC—chemokine (C—X—C |
| factor receptor | motif) ligand |
| Hb—haemoglobin | β2 Microglobin |
| Zinc | Cancer Antigen 19.9 |
| IL.17—interleukin - 17 | Eotaxin |
| VCAM-1—vascular cell | MIP-1-α—chemokine (C-C |
| adhesion molecule 1 | motif) ligand 3 |
| Cortisol | IGF.BP.2—insulin-like |
| | growth factor binding protein 2 |
| Pancreatic Polypeptide | ApoE ECU—apolipoprotein E |
| ABeta 42 | |

and naturally-occurring variants thereof.

59. The method of claim 58, further comprising comparing a measured level of at least another biomarker marker in a biological sample from the individual to a reference level for the at least another biomarker, wherein the at least another biomarker is selected from a panel of markers consisting of:

| | |
|---|---|
| alb/tpr | MPO—Myeloperoxidase |
| CD40—CD40 molecule | Neut—neutrophils |
| Chromium isotope | PCV—packed cell volume |
| 52/Chromium isotope 53 | |
| FT3 | Rb85—Rubidium |
| HCY—homocysteine | RCC—red cell count |
| IL.10—interleukin 10 | rFol—red cell folate |
| MCHC—mean cell | Selenium |
| haemoglobin concentration | |
| MMP.2—matrix | TNF.RII—Tumor necrosis |
| metallopeptidase 2 (72 kDa | factor receptor superfamily |
| type IV collagenase | member 1B |
| EGFR—Epidermal growth | tPr (total protein) |
| factor receptor | |
| Hepatocyte Growth Factor | VEGF Vascular endothelial |
| (HGF) | growth factor |
| ICAM-1—Intercellular | ANG-2—Angiopoietin-2 |
| adhesion molecule 1 | |
| TNF receptor superfamily | α-2-macroglobulin |
| member 5 | |
| Triiodothyronine | Calcium Corrected (Ca corr = |
| | Ca total + ((40 − alb) * |
| | 0.02)) |
| Apolipoprotein E4 Allelle | |

and naturally-occurring variants thereof.

60. The method according to claim 57, wherein comparing the measured level of the at least six biomarkers in the biological sample from the individual comprises comparing the measured level of any one of a set of six markers selected from the group comprising:
i) Abeta42, ApoE, VCAM1, BLC—chemokine (C-X-C motif) ligand, Pancreatic polypeptide, IL-17, or their naturally-occurring variants thereof;
ii) Abeta42, ApoE, Cortisol, BLC—chemokine (C-X-C motif) ligand, Pancreatic polypeptide, IL-17, or their naturally-occurring variants thereof;
iii) Abeta42, ApoE, Cortisol, VCAM1, Pancreatic polypeptide, IL-17, or their naturally-occurring variants thereof, or their naturally-occurring variants thereof;
iv) Abeta42, ApoE, Cortisol, VCAM1, BLC—chemokine (C-X-C motif) ligand, IL-17, or their naturally-occurring variants thereof; and
v) Abeta42, ApoE, Cortisol, VCAM1, BLC—chemokine (C-X-C motif) ligand, Pancreatic polypeptide, or their naturally-occurring variants thereof

61. The method according to claim 57, wherein the neurological disorder is Alzheimer's disease.

62. The method according to claim 57 wherein the biological sample is plasma.

63. The method according to claim 57, wherein the comparing of a measured level of at least six biomarkers in a biological sample from an individual to a reference level for the at least six biomarkers is carried out by one or more of the statistical methods selected from the group consisting of Random Forest, Support Vector Machine, Linear Models for MicroArray data (LIMMA) and/or Significance Analyses of Microarray Data (SAM), Best First, Greedy Stepwise, Naive Bayes, Linear Forward Selection, Scatter Search, Linear Discriminant Analysis (LDA), Stepwise Logistic Regression, Receiver Operating Characteristic and Classification Trees (CT).

64. The method according to claim 57, wherein comparing the measured levels for each biomarker is carried out using Boosted Trees (BT) and wherein the method provides sensitivity of at least 85% and specificity of at least 85% in diagnosing or aiding diagnosis of a neurological disorder in an individual.

65. A method for assessing the efficacy of treatment modalities of a neurological disorder in an individual or a population of individuals, the method comprising comparing a measured level of at least six biomarkers in a biological sample from an individual to a reference level for the at least six biomarkers, wherein the at least six biomarkers are selected from a panel of markers consisting of:
Cortisol
IGF.BP.2—insulin-like growth factor binding protein 2
IL.17—interleukin—17
Pancreatic Polypeptide
ApoE ECU—apolipoprotein E
ABeta 42
VCAM−1—vascular cell adhesion molecule
BLC—chemokine (C-X-C motif) ligand
and naturally-occurring variants thereof.

66. The method according to claim 65, further comprising comparing a measured level of at least one other biomarker in a biological sample from the individual to a reference level for the at least one other biomarker, wherein the at least one other biomarker is selected from a panel of markers consisting of:

| | |
|---|---|
| Alb—albumin | SOD—superoxide dismutase |
| B2M—beta-2-microglobulin | TIMP-1—tissue inhibitor of |
| | metalloproteinase 1 |
| CEA—carcinoembryonic | Adiponectin |
| antigen | |
| EGF.R—epidermal growth | BLC—chemokine (C—X—C |
| factor receptor | motif) ligand |
| Hb—haemoglobin | β2 Microglobin |
| Zinc | Cancer Antigen 19.9 |
| IL.17—interleukin - 17 | Eotaxin |
| VCAM-1—vascular cell | MIP-1-α—chemokine (C-C |
| adhesion molecule 1 | motif) ligand 3 |
| Cortisol | IGF.BP.2—insulin-like |
| | growth factor binding protein 2 |
| Pancreatic Polypeptide | ApoE ECU—apolipoprotein E |
| ABeta 42 | |

and naturally-occurring variants thereof.

67. The method according to claim 66, further comprising comparing a measured level of at least another biomarker marker in a biological sample from the individual to a reference level for the at least another biomarker, wherein the at least another biomarker is selected from a panel of markers consisting of:

| | |
|---|---|
| alb/tpr | MPO—Myeloperoxidase |
| CD40—CD40 molecule | Neut—neutrophils |
| Chromium isotope | PCV—packed cell volume |
| 52/Chromium isotope 53 | |
| FT3 | Rb85—Rubidium |
| HCY—homocysteine | RCC—red cell count |
| IL.10—interleukin 10 | rFol—red cell folate |
| MCHC—mean cell | Selenium |
| haemoglobin concentration | |
| MMP.2—matrix | TNF.RII—Tumor necrosis |
| metallopeptidase 2 (72 kDa | factor receptor superfamily |
| type IV collagenase | member 1B |
| EGFR—Epidermal growth | tPr (total protein) |
| factor receptor | |
| Hepatocyte Growth Factor | VEGF Vascular endothelial |
| (HGF) | growth factor |
| ICAM-1—Intercellular | ANG-2—Angiopoietin-2 |
| adhesion molecule 1 | |
| TNF receptor superfamily | α-2-macroglobulin |
| member 5 | |
| Triiodothyronine | Calcium Corrected (Ca corr = |
| | Ca total + ((40 − alb) * |
| | 0.02)) |
| Apolipoprotein E4 Allelle | |

and naturally-occurring variants thereof.

68. The method according to claim 65, wherein comparing the measured level of the at least six biomarkers in the biological sample from the individual comprises comparing the measured level of any one of a set of six markers selected from the group comprising:
i) Abeta42, ApoE, VCAM1, BLC—chemokine (C-X-C motif) ligand, Pancreatic polypeptide, IL-17, or their naturally-occurring variants thereof;
ii) Abeta42, ApoE, Cortisol, BLC—chemokine (C-X-C motif) ligand, Pancreatic polypeptide, IL-17, or their naturally-occurring variants thereof;
iii) Abeta42, ApoE, Cortisol, VCAM1, Pancreatic polypeptide, IL-17, or their naturally-occurring variants thereof, or their naturally-occurring variants thereof;
iv) Abeta42, ApoE, Cortisol, VCAM1, BLC—chemokine (C-X-C motif) ligand, IL-17, or their naturally-occurring variants thereof; and
v) Abeta42, ApoE, Cortisol, VCAM1, BLC—chemokine (C-X-C motif) ligand, Pancreatic polypeptide, or their naturally-occurring variants thereof

69. The method of claim 65, wherein the neurological disorder is Alzheimer's disease.

70. The method of claim 65 wherein the biological sample is plasma.

71. The method of claim 65, wherein the comparing of a measured level of at least four biomarkers in a biological sample from an individual to a reference level for the at least six biomarkers is carried out by one or more of the statistical methods selected from the group consisting of Random Forest, Support Vector Machine, Linear Models for MicroArray data (LIMMA) and/or Significance Analyses of Microarray Data (SAM), Best First, Greedy Stepwise, Naive Bayes, Linear Forward Selection, Scatter Search, Linear Discriminant Analysis (LDA), Stepwise Logistic Regression, Receiver Operating Characteristic and Classification Trees (CT).

72. The method of claim 65, wherein comparing the measured levels for each biomarker is carried out using Boosted Trees (BT) and wherein the method provides sensitivity of at least 85% and specificity of at least 85% in diagnosing or aiding diagnosis of a neurological disorder in an individual.
